(19) United States
(12) Patent Application Publication
(10) Pub. No.: US 2025/0145644 A1
(43) Pub. Date: May 8, 2025

(54) PROCESSES AND COMPOUNDS FOR PREPARING SPIROLIGOMERS

(71) Applicants: Temple University-Of The Commonwealth System of Higher Education, Philadelphia, PA (US); ThirdLaw Molecular Inc., Blue Bell, PA (US)

(72) Inventors: Christian Schafmeister, Merion Station, PA (US); Justin Northrup, Langhorne, PA (US)

(21) Appl. No.: 18/836,776

(22) PCT Filed: Feb. 8, 2023
(86) PCT No.: PCT/US2023/062175

§ 371 Date: Aug. 8, 2024

Related U.S. Application Data
(60) Provisional application No. 63/307,869, filed on Feb. 8, 2022.

(51) Int. Cl. C07F 1/08; C07D 207/16; C07D 401/14; C07D 487/22

(52) U.S. Cl. CPC C07F 1/08; C07D 207/16; C07D 401/14; C07D 487/22

(57) ABSTRACT

The present invention provides novel intermediates and other compounds prepared using compounds of Formula I. These novel compounds include spiroligomers that have particular significance in the peptide industry. The processes described herein provide products that are produced in high yields in high purities and, thus, are highly efficient.

### CROSS-REFERENCE TO RELATED APPLICATIONS

This application claims priority to and the benefit of U.S. Provisional Application No. 63/307,869, filed Feb. 8, 2022, the disclosure of which is incorporated herein by reference herewith in its entirety.

### STATEMENT REGARDING FEDERALLY SPONSORED RESEARCH OR DEVELOPMENT

This invention was made with government under DE-EE0008321 awarded by the U.S. Department of Energy and HDTRA1-16-1-0047 awarded by the Defense Threat Reduction Agency. The government has certain rights in the invention.

### TECHNICAL FIELD

The disclosure is directed to processes and compounds for preparing spiroligomers.

### BACKGROUND OF THE INVENTION

Bis-peptides, also known as spiroligomers, are ladder oligomers or polymers formed from amino acids. Spiroligomers are useful as therapeutics, catalysts, sensors, mimics of antibodies, and nanotechnology. Their shape can be tailored based on stereochemistry and sequence of the monomers. In synthesizing bis-peptides, diketopiperazine rings are formed using amino acids to create spiroligomers.

The standard practice for synthesizing spiroligomers is via a step-wise approach by sequentially adding a single bis-amino acid at each stage of the synthesis. Although processes of this type permit stereochemical control, they are relatively inefficient. For example, such processes typically produce no more than a few milligrams of spiroligomer, the steps are difficult to perform, and the processes often require long periods of time and a great deal of labor.

Thus, there is a need in the art for effective methods for preparing spiroligomers via simple steps and/or in high yields. The present invention addresses this unmet need in the art.

### SUMMARY OF THE INVENTION

In some aspects, the present invention provides processes comprising contacting a compound of Formula I with R¹X, R¹C(O)H, or R¹C(O)R²:

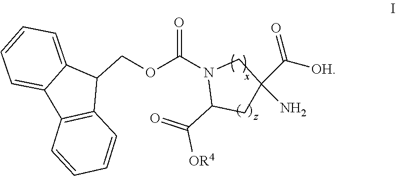

wherein, R¹, R², X, R⁴, x, and z are defined herein; in the presence of a reducing agent; for a time and under conditions effective to produce a compound of Formula II:

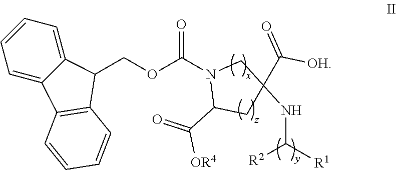

wherein, y is an integer 0 or 1; with the proviso that when the compound of Formula I is reacted with R¹X, y is an integer 0 and R² is absent.

In other aspect, the present invention provides compounds, such as spiroligomers, produced using the processes described herein.

In further aspects, the present invention provides processes for preparing a compound of Formula I, III, IV, VII, and XV, wherein R¹, R², R⁴, R⁵, x, y, and z are defined herein:

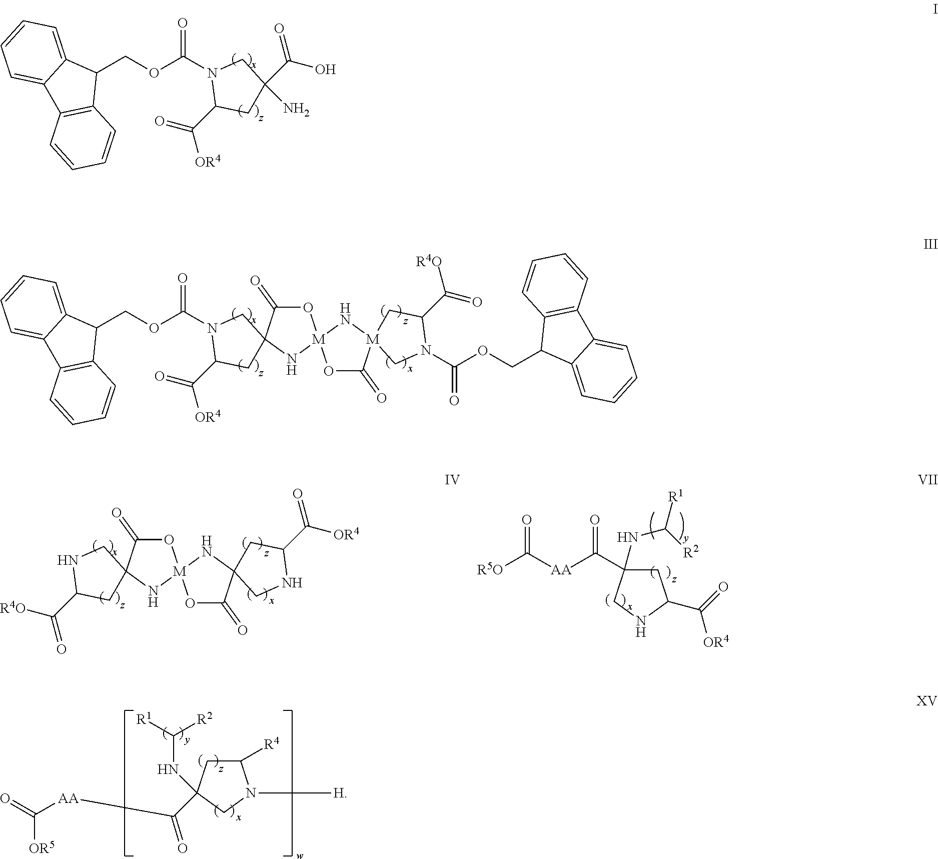

In still other aspect, the present invention provides compounds of Formula II, wherein R¹, R², R⁵, and y are defined herein:

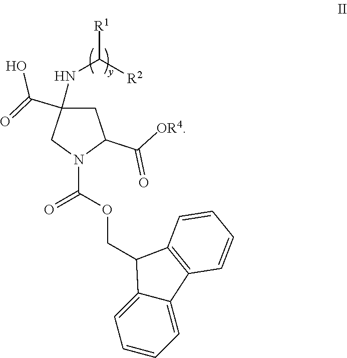

or a pharmaceutically acceptable salt thereof.

In yet other aspects, the present invention provides compounds such as:

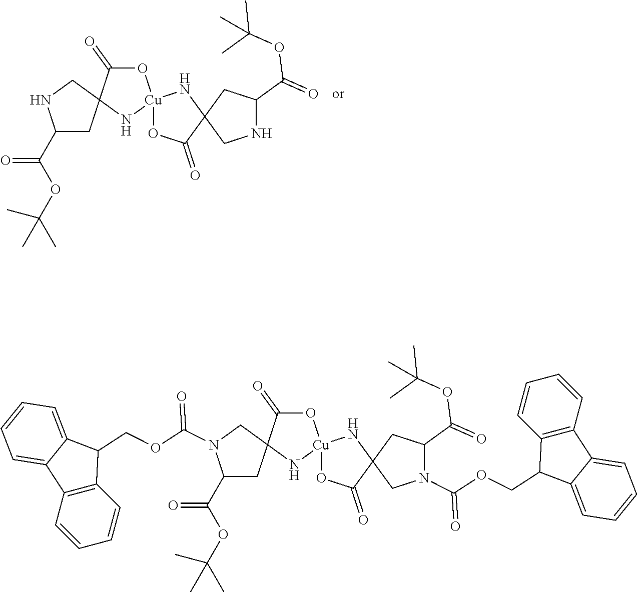

Other aspects and embodiments of the invention will be readily apparent from the following detailed description of the invention.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a schematic representation of the fluorenylmethoxycarbonyl (Fmoc)-protection of functionalized bis-amino acid building blocks using a temporary Cu²⁺ complexation strategy, together with an efficient multi-kilogram-scale synthesis of bis-amino acid precursors.

FIG. 2 depicts a schematic representation of the synthesis of Cbz building blocks.

FIG. 3 depicts a schematic representation of the synthesis of Fmoc building blocks.

FIG. 4 depicts a schematic representation of the synthesis of functionalized building blocks and Pfp esters.

FIG. 5 depicts schematic representations of the chemical structures of spiroligomer (a) T2, (b) T3, (c) T4, and (d) the modeled structure of T1 based on ROESY correlations and GAFF energy minimization by CANDO is shown

FIG. 6 depicts a schematic representation of the synthesis of the functionalized Spiroligomer T1 with selected ROESY correlation and modeled structure.

FIG. 7 depicts a schematic representation of compound S2a, which was previously reported (Levins, C. G. et al., Journal of the American Chemical Society 2003, 125 (16), 4702-4703), and the corresponding representative ¹H NMR spectrum.

FIG. 8 depicts a schematic representation of compound S3a, which was previously reported (Levins, C. G. et al., Journal of the American Chemical Society 2003, 125 (16), 4702-4703), and the corresponding representative ¹H NMR spectrum.

FIG. 9 depicts a schematic representation of compound 2a, which was previously reported (Levins, C. G. et al., Journal of the American Chemical Society 2003, 125 (16), 4702-4703), and the corresponding representative ¹H NMR spectrum.

FIG. 10 depicts a schematic representation of compound S2b, which was previously reported (Levins, C. G. et al., Journal of the American Chemical Society 2003, 125 (16), 4702-4703), and the corresponding representative ¹H NMR spectrum.

FIG. 11 depicts a schematic representation of compound S3b, which was previously reported (Levins, C. G. et al., Journal of the American Chemical Society 2003, 125 (16), 4702-4703), and the corresponding representative ¹H NMR spectrum.

FIG. 12 depicts a schematic representation of compound 2b, which was previously reported (Levins, C. G. et al., Journal of the American Chemical Society 2003, 125 (16), 4702-4703), and the corresponding representative ¹H NMR spectrum.

FIG. 13 depicts a schematic representation of compound 4a (3S,5S)-1-(((9H-fluoren-9-yl)methoxy)carbonyl)-3-amino-5-(tert-butoxycarbonyl)pyrrolidine-3-carboxylic acid and the corresponding representative ¹H NMR and ¹³C NMR spectra.

FIG. 14 depicts a schematic representation of compound 4b (3R,5S)-1-(((9H-fluoren-9-yl)methoxy)carbonyl)-3-amino-5-(tert-butoxycarbonyl)pyrrolidine-3-carboxylic acid and the corresponding representative ¹H NMR and ¹³C NMR spectra.

FIG. 15 depicts a schematic representation of compound 4c (3R,5R)-1-(((9H-fluoren-9-yl)methoxy)carbonyl)-3-amino-5-(tert-butoxycarbonyl)pyrrolidine-3-carboxylic acid and the corresponding representative ¹H NMR and ¹³C NMR spectra.

FIG. 16 depicts a schematic representation of compound 4d (3R,5S)-1-(((9H-fluoren-9-yl)methoxy)carbonyl)-3-amino-5-(tert-butoxycarbonyl)pyrrolidine-3-carboxylic acid and the corresponding representative ¹H NMR and ¹³C NMR spectra.

FIG. 17 depicts a schematic representation of compound 5a (3S,5S)-1-(((9H-fluoren-9-yl)methoxy)carbonyl)-5-(tert-butoxycarbonyl)-3-((naphthalen-2-ylmethyl)amino) pyrrolidine-3-carboxylic acid and the corresponding representative ¹H NMR and ¹³C NMR spectra.

FIG. 18 depicts a schematic representation of bis amino acid 5b (3R,5S)-1-(((9H-fluoren-9-yl)methoxy)carbonyl)-5-(tert-butoxycarbonyl)-3-(isopentylamino) pyrrolidine-3-carboxylic acid and the corresponding representative ¹H NMR and ¹³C NMR spectra.

FIG. 19 depicts a schematic representation of compound 5c (3R,5R)-1-(((9H-fluoren-9-yl)methoxy)carbonyl)-5-(tert-butoxycarbonyl)-3-((pyridin-4-ylmethyl)amino) pyrrolidine-3-carboxylic acid and the corresponding representative ¹H NMR and ¹³C NMR spectra.

FIG. 20 depicts a schematic representation of compound 5d (3S,5R)-1-(((9H-fluoren-9-yl)methoxy)carbonyl)-5-(tert-butoxycarbonyl)-3-((3,4-dichlorobenzyl)amino) pyrrolidine-3-carboxylic acid and the corresponding representative ¹H NMR and ¹³C NMR spectra.

FIG. 21 depicts a schematic representation of compound 6a 1-((9H-fluoren-9-yl)methyl)2-(tert-butyl)4-(perfluorophenyl)(2S,4S)-4-((naphthalen-2-ylmethyl)amino) pyrrolidine-1,2,4-tricarboxylate and the corresponding representative ¹H NMR and ¹³C NMR spectra.

FIG. 22 depicts a schematic representation of compound 6b 1-((9H-fluoren-9-yl)methyl)2-(tert-butyl)4-(perfluorophenyl)(2S,4R)-4-(isopentylamino) pyrrolidine-1,2,4-tricarboxylate and the corresponding representative ¹H NMR and ¹³C NMR spectra.

FIG. 23 depicts a schematic representation of compound 6c 1-((9H-fluoren-9-yl)methyl)2-(tert-butyl)4-(perfluorophenyl)(2R,4R)-4-((pyridin-4-ylmethyl)amino) pyrrolidine-1,2,4-tricarboxylate and the corresponding representative ¹H NMR and ¹³C NMR spectra.

FIG. 24 depicts a schematic representation of compound 6d 1-((9H-fluoren-9-yl)methyl)2-(tert-butyl)4-(perfluorophenyl)(2R,4S)-4-((3,4-dichlorobenzyl)amino) pyrrolidine-1,2,4-tricarboxylate and the corresponding representative ¹H NMR and ¹³C NMR spectra.

FIG. 25 depicts representative images of Fmoc building block synthesis during (a) hydrogenolysis of Cbz building block with Pd/C; (b) Fmoc protection with Fmoc-Cl; (c) removal of Pd/C with celite column; (d) removal of Cu²⁺ with EDTA solution and precipitation of product; (d) isolation of product by vacuum filtration.

FIG. 26 depicts representative HPLC chromatograph of crude T1 at 220 nm without purification.

FIG. 27 depicts representative QTOF LCMS results of T1: (top) LC chromatogram at 220 nm; (middle) total ion current chromatogram; (bottom) mass spectrogram with theoretical and observed monoisotopic peaks in red and black, respectively.

FIG. 28 depicts representative QTOF LCMS results of T2: (top) LC chromatogram at 220 nm; (middle) total ion current chromatogram; (bottom) mass spectrogram with theoretical and observed monoisotopic peaks in red and black, respectively.

FIG. 29 depicts representative QTOF LCMS results of T3: (top) LC chromatogram at 220 nm; (middle) total ion current chromatogram; (bottom) mass spectrogram with theoretical and observed monoisotopic peaks in red and black, respectively.

FIG. 30 depicts representative QTOF LCMS results of T4: (top) LC chromatogram at 220 nm; (middle) total ion current chromatogram; (bottom) mass spectrogram with theoretical and observed monoisotopic peaks in red and black, respectively.

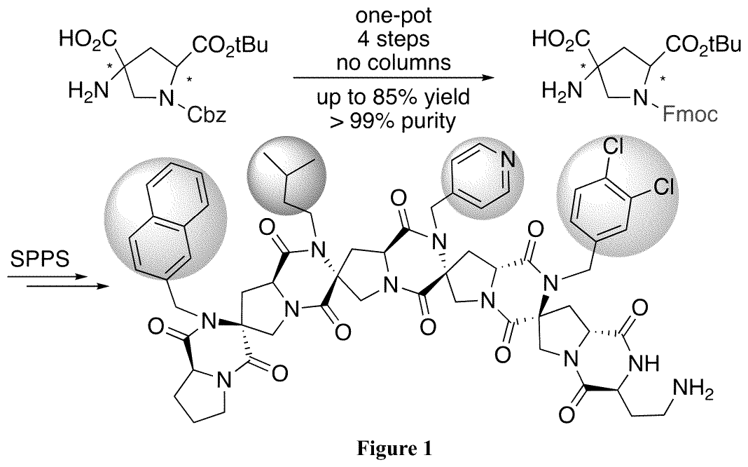

### DETAILED DESCRIPTION OF THE INVENTION

The present invention provides efficient processes for preparing spiroligomers. As opposed to the processes in the art, the processes described herein permit the preparation of larger quantities of spiroligomer that have highly preorganized structures. By doing so, a large variety of building blocks in different length sequences may be assembled to result in an almost infinite number of highly pre-organized molecules with programmable shape and functional group display.

The present invention uses key and unique steps that provide the ability to prepare the spiroligomers and intermediates thereof in high yields. Initially, the processes permitted the preparation of the intermediates in 40-50 gram batches, which was far in excess of the yields described in the art. After refining the processes, multi-kilogram batches of the intermediates and products could be prepared.

Among other features, one unique step includes the use of the delicate Fmoc group to protect an amine at a key location of the molecule. A variety of protecting groups have been used to date in processes for preparing spiroligomers. However, the inventors found that simply swapping out the traditional protecting groups for a Fmoc group was not efficient and reduced in a much lower yield of spiroligomer, for which the yield already was low. The Fmoc protected group also could easily be functionalized via straightforward steps using a large range and number of functional groups. Another key step includes the use of metals, such as copper, to selectively complex amino acid groups to permit the selective protection of other amines with the Fmoc group. These key steps resulted in high yields, i.e., >90%, and purities of greater than 95%.

Finally, the spiroligomers, once formed, could smoothly be rigidified using non-stringent conditions for minimal periods of time.

### Definitions

In the present invention the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. In some aspects of the present invention, “about” refers to a range of values that is ±10% of the recited value. For example, “about 10,” refers to “9 to 11,” as well as “10.” The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range. It is understood that where a parameter range is provided, all integers within that range, and tenths thereof, are also provided by the invention. For example, “20-40 mg” includes 20.0 mg, 20.1 mg, 20.2 mg, 20.3 mg, etc. up to 40.0 mg.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

The term “halo” as used herein refers to Cl, F, Br, or I. In some aspects, halo is Cl. In other aspects, halo is F. In further aspects, halo is Br. In yet other aspects, halo is I.

“C0” as used herein refers to the absence of a carbon atom. For example, C₀₋₆ alkylOH refers to —OH and C₁₋₆ alkyl.

The term “alkyl” as used herein refers to an aliphatic hydrocarbon containing one to twelve carbon atoms, i.e., C₁₋₁₂alkyl. In some embodiments, the alkyl is methyl, ethyl, propyl, butyl, pentyl, hexyl, heptyl, octyl, nonyl, decyl, etc. In other embodiments, the alkyl is C₁₋₈ alkyl. In further embodiments, the alkyl is C₁₋₆ alkyl. In still further embodiments, the alkyl is C₁₋₄ alkyl. In yet other embodiments, the alkyl is C₁₋₄ alkyl. Examples of alkyl groups include methyl, ethyl, propyl (e.g., n-propyl, isopropyl), butyl (e.g., sec-butyl, tert-butyl, iso-butyl), 3-pentyl, hexyl, heptyl, octyl, nonyl, and decyl. The alkyl may be unsubstituted or substituted, i.e., optionally substituted, as described herein. In certain aspects, the alkyl is substituted with two substituents. In further aspects, the alkyl is substituted with one substituent. In other aspects, the alkyl is substituted with three substituents. In still further aspects, the alkyl is unsubstituted.

The term “alkenyl” as used herein refers to an alkyl containing one or more carbon-carbon double bonds. In some embodiments, an alkenyl group contains one, two or three carbon-carbon double bonds. In other embodiments, the alkenyl contains one carbon-carbon double bond. In further embodiments, the alkenyl is a C₂₋₆ alkenyl. In yet other embodiments, the alkenyl is a C₂₋₄ alkenyl. In still further embodiments, the alkenyl is a C₃₋₄ alkenyl. Non-limiting exemplary alkenyl groups include ethenyl, propenyl, isopropenyl, butenyl, sec-butenyl, pentenyl, hexenyl, and —CH═C(CH₃)₂. The alkenyl may be unsubstituted or substituted. In some embodiments, the alkenyl is substituted with two substituents. In further embodiments, the alkenyl is substituted with one substituent. In yet other embodiments, the alkenyl is substituted with three substituents. In still further embodiments, the alkenyl is unsubstituted.

The term “alkynyl” as used herein refers to an alkyl containing one or more carbon-carbon triple bonds. In some embodiments, an alkynyl group contains one, two or three carbon-carbon triple bonds. In other embodiments, the alkynyl contains one carbon-carbon triple bond. In further embodiments, the alkynyl is a C₂₋₆ alkynyl. In yet other embodiments, the alkynyl is a C₂₋₄ alkynyl. In still further embodiments, the alkynyl is a C₃₋₄ alkynyl. Non-limiting exemplary alkynyl groups include ethynyl, propynyl, butynyl, pentynyl, hexynyl, among others. The alkynyl may be unsubstituted or substituted. In some embodiments, the alkynyl is substituted with two substituents. In further embodiments, the alkynyl is substituted with one substituent. In yet other embodiments, the alkynyl is substituted with three substituents. In still further embodiments, the alkynyl is unsubstituted.

The term “alkoxy” as used herein refers to an optionally substituted alkyl as defined herein that contains an oxygen atom within the group. In some embodiments, the alkoxy group is an alkyl attached to a terminal oxygen atom. In other embodiments, the alkoxy group is C₁₋₆ alkoxy. In further embodiments, the alkoxy group is C₁₋₄ alkoxy. Examples of alkoxy groups include methoxy (OCH₃), ethoxy (OCH₂CH₃ or CH₂OCH₃), propoxy (e.g., —OⁿPr, —OⁱPr), or butoxy (e.g., —OⁿBu, —OⁱBu, —O^{s}Bu, —O^{t}Bu). The alkoxy may be unsubstituted or substituted. In some embodiments, the alkoxy is substituted with two substituents. In further embodiments, the alkoxy is substituted with one substituent. In yet other embodiments, the alkoxy is substituted with three substituents. In still further embodiments, the alkoxy is unsubstituted.

The term “cycloalkyl” as used herein refers to a saturated or partially unsaturated cyclic aliphatic hydrocarbon containing one to three rings and three to twelve carbon atoms, i.e., C₃₋₁₂ cycloalkyl or C₃₋₁₂cycloalkenyl. In some embodiments, the cycloalkyl has two rings. In other embodiments, the cycloalkyl has one ring. In further embodiments, the cycloalkyl is saturated. In yet other embodiments, the cycloalkyl has one or two double bonds. In still further embodiments, the cycloalkyl is C₃₋₈ cycloalkyl. In yet further embodiments, the cycloalkyl is C₃₋₇cycloalkyl. In still other embodiments, the cycloalkyl is C₃₋₇cycloalkenyl. In other embodiments, the cycloalkyl is C₃₋₆ cycloalkyl. In further embodiments, the cycloalkyl is C₃₋₆ cycloalkyl. Examples of cycloalkyl groups include cyclopropyl, cyclobutyl, cyclopentyl, cyclohexyl, cycloheptyl, cyclooctyl, norbornyl, decalin, adamantyl, cyclohexenyl, and cyclopentenyl, among others. A cycloalkyl may be unsubstituted or substituted. In some embodiments, the cycloalkyl is substituted with two substituents. In further embodiments, the cycloalkyl is substituted with one substituent. In yet other embodiments, the cycloalkyl is substituted with three substituents. In still further embodiments, the cycloalkyl is unsubstituted.

The term “cyanoalkyl” as used herein refers to an alkyl group as described that is substituted with one or more CN. In some embodiments, the cyanoalkyl contains one CN substituent. In other embodiments, the cyanoalkyl contains two CN substituents. Examples of C₁₋₆ cyanoalkyl include, without limitation, CH₂CN, CH₂CH₂CN, CHCNCH₃.

The term “haloalkyl” as used herein refers to an alkyl group as described that is substituted with one or more halo. In some embodiments, the haloalkyl contains one or more F, i.e., fluoroalkyl. In other embodiments, the haloalkyl contains one halo (e.g., F). In further embodiments, the haloalkyl contains two halo (e.g., F). In yet other embodiments, the haloalkyl contains three halo (e.g., F). Examples of C₁₋₆ haloalkyl include, without limitation, CH₂F, CHF₂, CF₃, CH₂CFH₂, CH₂CF₂H, CH₂CH₂CF₃, among others.

The term “haloalkoxy” as used herein refers to an alkoxy group as described that is substituted with one or more halo. In some embodiments, the haloalkoxy contains one or more F, i.e., fluoroalkyl. In other embodiments, the haloalkoxy contains one halo (e.g., F). In further embodiments, the haloalkoxy contains two halo (e.g., F). In yet other embodiments, the haloalkoxy contains three halo (e.g., F). Examples of C₁₋₆ haloalkoxy include, without limitation, OCH₂F, OCHF₂, OCF₃, OCH₂CFH₂, OCH₂CF₂H, OCH₂CH₂CF₃, among others.

The term “aryl” as used herein refers to a monocyclic or bicyclic unsaturated ring system having 5-14 carbon atoms, i.e., a C₅₋₁₄ aryl. In some embodiments, the aryl has 6-12 carbon atoms, i.e., C₆₋₁₂aryl. In further embodiments, the aryl has 6-10 carbon atoms, i.e., C₆₋₁₀aryl. In other embodiments, the aryl has 6-8 carbon atoms, i.e., C₆₋₈ aryl. Examples of aryl groups include, without limitation, phenyl, naphthyl (1-naphthyl, 2-naphthyl), phenanthryl, anthracyl, indenyl, azulenyl, biphenyl, biphenylenyl, and fluorenyl groups. In some embodiments, the aryl group is phenyl or naphthyl. The term “aryl” also includes phenyl groups fused to a cycloalkyl. The aryl may be unsubstituted or substituted. In some embodiments, the aryl is an optionally substituted phenyl.

The term “heteroaryl” as use herein refers to monocyclic or bicyclic aromatic ring systems having 5 to 14 ring atoms, i.e., a 5- to 14 membered heteroaryl. The heteroaryl contains carbon atoms and one or more of a heteroatom that is oxygen, nitrogen and sulfur. In some embodiments, the heteroaryl contains 1, 2, 3, or 4 oxygen, nitrogen and/or sulfur. In other embodiments, the heteroaryl contains three heteroatoms. In further embodiments, the heteroaryl contains two heteroatoms. In yet other embodiments, the heteroaryl contains one heteroatom. In yet other embodiments, the heteroaryl is a 5- to 10 membered heteroaryl. In still further embodiments, the heteroaryl is a 5- or 6 membered heteroaryl. In other embodiments, the heteroaryl is 5-membered. In further embodiments, the heteroaryl is 6-membered. Examples of heteroaryl groups include thienyl (e.g., thien-2-yl, thien-3-yl), benzo[b]thienyl, naphtho[2,3-b]thienyl, thianthrenyl, furyl (e.g., 2-furyl, 3-furyl, 4-furyl), benzofuryl, pyranyl, thiophenyl, benzofuranyl, isobenzofuranyl, benzooxazonyl, chromenyl, xanthenyl, pyrrolyl (e.g., pyrrol-2-yl, pyrrol-3-yl), imidazolyl (e.g., imidazol-2-yl, imidazol-4-yl, 1-methylimidazolyl), pyrazolyl (e.g., pyrazol-3-yl, pyrazol-4-yl, pyrazol-5-yl), pyridyl (e.g., pyridin-1-yl, pyridin-2-yl, pyridin-3-yl, pyridin-4-yl), pyrazinyl (e.g., pyrazin-2-yl, pyrazin-3-yl, pyrazin-5-yl, pyrazin-6-yl), pyrimidinyl (e.g., pyrimidin-2-yl, pyrimidin-4-yl, pyrimidin-5-yl), pyridazinyl, isoindolyl, 3H-indolyl, indolyl, indazolyl, purinyl, isoquinolyl, quinolyl, phthalazinyl, naphthyridinyl (e.g., 1,8-naphthyridinyl), cinnolinyl, triazolyl (e.g., 1,2,4-triazol-1-yl, 1,2,4-triazol-3-yl, 1,2,4-triazol-5-yl), thiadiazolyl (e.g., 1,2,3-thiadiazolyl, 1,2,4-thiadiazolyl, 1,2,5-thidiazolyl, 1,3,4-thiadiazolyl), oxadiazolyl (e.g., 1,2,3-oxadiazolyl, 1,2,4-oxadiazolyl, 1,2,5-oxadiazolyl, 1,3,4-oxadiazolyl), quinazolinyl, pteridinyl, 4 H-carbazolyl, carbazolyl, β-carbolinyl, phenanthridinyl, acridinyl, phenanthrolinyl, phenazinyl, thiazolyl (e.g., thiazol-2-yl, thiazol-4-yl, thiazol-5-yl, 2-methylthiazoyl), isothiazolyl (e.g., isothiazol-3-yl, isothiazol-4-yl, isothiazol-5-yl), phenothiazolyl, isoxazolyl (e.g., isoxazol-3-yl, isoxazol-4-yl, isoxazol-5-yl, 3,5-dimethylisoxazoyl), furazanyl, pyrazolo[1,5-a]pyridinyl, benzoisothiazolyl, imidazol[1,5-a]pyridinyl (e.g., imidazol[1,5-a]pyridin-1-yl), pyrrolo[1,2]pyridazinyl (e.g., pyrrolo[1,2]pyridazin-5-yl, pyrrolo[1,2]pyridazin-6-yl), benzo[d]thiazolyl (e.g., benzo[d]thiazol-3-yl, benzo[d]thiazol-2-yl), benzo[d]imidazolyl (e.g., benzo[d]imidazol-2-yl), benzo[d]oxazolyl (e.g., benzo[d]oxazol-2-yl), benzo[c]isoxazolyl (e.g., benzo[c]isoxazol-3-yl), isothiazolyl, 4,5,6,7-tetrahydropyrazolo[1,5-a]pyridinyl, phenoxazinyl, triazinyl, indolizinyl, quinolinyl, 1,2,3,4-tetrahydroquinolinyl, isoquinolinyl, 1,2,3,4-tetrahydroisoquinolinyl, benzo[b]thiophenyl, 1H-indazolyl (e.g., indazol-3-yl), benzthiazolyl, 4 H-quinolizinyl, quinoxalinyl, phenothiazinyl, oxazolyl (e.g., oxazol-2-yl, oxazol-4-yl, oxazol-5-yl), or 2-pyrenyl. The term “heteroaryl” is also include N-oxides. The heteroaryl may be unsubstituted or substituted. In some embodiments, the heteroaryl is substituted with two substituents. In further embodiments, the heteroaryl is substituted with one substituent. In yet other embodiments, the heteroaryl is substituted with three substituents. In still further embodiments, the heteroaryl is unsubstituted. Substitution may occur on any available carbon or heteroatom (e.g., nitrogen), or both, as permitted by substituent valency. The heteroaryl also includes heteroaryl groups having a fused optionally substituted cycloalkyl or fused optionally substituted heterocyclyl.

The term “heterocyclyl” as used herein refers to non-aromatic, saturated or partially unsaturated groups containing one, two, or three rings having from three to fourteen ring members, i.e., a 3-14-membered heterocyclyl. The heterocyclyl group contains carbon atoms and one or more of oxygen, sulfur, and/or nitrogen atoms, which can be oxidized or quaternized. In some embodiments, a ring CH₂— is replaced with a —C(═O)—. The term “heterocyclyl” also includes groups having fused optionally substituted aryl groups, e.g., indolinyl or chroman-4-yl. In some embodiments, the heterocyclyl group is a C₄₋₆ heterocyclyl. The heterocyclyl can be optionally linked to the rest of the molecule through any available carbon or heteroatom. Examples of heterocyclyls include azetidinyl (e.g., azetidin-1-yl, azetidin-2-yl, azetidin-3-yl), dioxanyl, tetrahydropyranyl, pyrrolidinyl, piperidinyl, morpholinyl, piperazinyl, pyrrolidinyl, indolinyl, oxetanyl, tetrahydrofuranyl, tetrahydrothiophenyl, 2-pyrazolinyl, pyrazolidinyl, trithianyl, indolizinyl, benzo[b]thiophenyl, 1H-indazolyl, benzthiazolyl, 4 H-quinolizinyl, quinolinyl, 1,2,3,4-tetrahydroquinolinyl, isoquinolinyl, 1,2,3,4-tetrahydroisoquinolinyl, quinoxalinyl, phenothiazinyl. The heterocyclyl may be unsubstituted or substituted. In some embodiments, the heterocyclyl is substituted with two substituents. In further embodiments, the heterocyclyl is substituted with one substituent. In yet other embodiments, the heterocyclyl is substituted with three substituents. In still further embodiments, the heterocyclyl is unsubstituted.

Any of the groups/substituents identified above or herein may be substituted with one or more of CN, halo, NO₂, OH, NH₂, C₁₋₆ alkoxy, C₁₋₆ alkyl, C₂₋₆ alkenyl, C₂₋₆ alkynyl, NH(C₁₋₆ alkyl), N(C₁₋₆ alkyl)(C₁₋₆ alkyl), aryl, O-aryl, C₁₋₆ alkylSH, —C(O)C₁₋₆ alkyl, —C(O)OC₁₋₆ alkyl, —C(O)aryl, —C(O)Oaryl, —C(O)heteroaryl, —C(O)Oheteroaryl, —C(O)heterocyclyl, —C(O)Oheterocyclyl, C₁₋₆ cyanoalkyl, C₃₋₈ cycloalkyl, C₁₋₆ haloalkoxy, C₁₋₆ haloalkyl, heteroaryl, heterocyclyl, —C₁₋₆ alkyl(heterocyclyl), —C₁₋₆ alkyl(aryl), —C₁₋₆ alkyl(heteroaryl), —C₁₋₆ alkyl(cycloalkyl), C₁₋₆ hydroxyalkyl, C(O)NH₂, or OC(O)NH₂.

The term “nucleobase” as used herein refers to optionally substituted adenine, cytosine, guanine, thymine, or uracil. In some embodiments, the nucleobase is a purine base such as adenine or guanine. In other embodiments, the nucleobase is a pyrimidine base such as cytosine, uracil, or thymine. In further embodiments, the nucleobase is a modified nucleobase such as a modified purine nucleobase or modified pyrimidine nucleobase. Examples of modified nucleobases include hypoxanthine, xanthine, 7-methylguanine, inosine, xanthosine, 7-methylguanosine, 5,6-dihydrouracil, 5-methylcytosine, 5-hydroxymethylcytosine, dihydrouridine, or 5-methylcytidine. In yet other embodiments, the nucleobase is an artificial nucleobase such as isoguanine, isocytosine, 2-amino-6-(2-thienyl) purine, or pyrrole-2-carbaldehyde.

The term “amino acid” as used herein refer to natural or non-natural amino acid. In some embodiments the amino acid is a natural amino acid. In certain aspects, the amino acid is arginine (Arg), histidine (His), lysine (Lys), aspartate (Asp), glutamate (Glu), serine (Ser), threonine (Thr), asparagine (Asn), glutamine (Gln), cysteine (Cys), selecocysteine (Sec), glycine (Gly), proline (Pro), alanine (Ala), valine (Val), isoleucine (Ile), leucine (Leu), methionine (Met), phenylalanine (Phe), tyrosine (Tyr), tryptophan (Trp), or pyrrolysine. In further embodiments, the amino acid is a non-natural amino acid. In certain embodiments, the amino acid is 4-aminobenzoic acid (PABA), alloisoleucine, allothreonine, carboxyglutamic acid, cystathionine, D-alanine, dehydroalanine, D-glutamate, diaminopimelic acid, djenkolic acid, glycine betaine homocysteine, homonorleucine, homoserine, hydroxyglycine, hydroxyproline, hypusine, isoserine, isovaline, lanthionine, N-ethyl alanine, N-ethyl glycine, N-ethyl β-alanine, N-isopropyl glycine, N-methyl alanine, N-methyl β-alanine, norleucine, norvaline, N-propyl glycine, O-methyl-homoserine, ornithine, pipecolic acid, pyroglutamic acid, sarcosine, selenocysteine, selenohomocysteine, selenomethionine, selenoethionine, taurine, t-leucine, α,β-diaminopropionic acid, α,γ-diaminobutyric acid, α-aminoisobutyric acid, α-amino-n-butyric acid, α-amino-n-heptanoic acid, α-hydroxy-γ-aminobutyric acid, β-alanine, β-aminoisobutyric acid, β-amino-n-butyric acid, γ-aminobutyric acid, δ-aminolevulinic acid, 1-aminocyclopropane-1-carboxylic acid, azetidine-2-carboxylic acid, cycloleucine, or pseudoproline. In some embodiments, the amino acid is protected with a suitable protecting group as described herein.

The term “sugar” as used herein refers to simple or compound sugars, as is known in the art. In some embodiments, the sugar is a monosaccharide such as glucose, fructose, or galactose. In other embodiments, the sugar is a disaccharide or double sugar (two monosaccharides joined by a glycosidic bond) such as sucrose, lactose, or maltose. In some embodiments, the sugar is protected with a suitable protecting group herein.

Ranges: throughout this present invention, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### The Processes

The present invention relates, in part, to processes preparing compounds of Formula II:

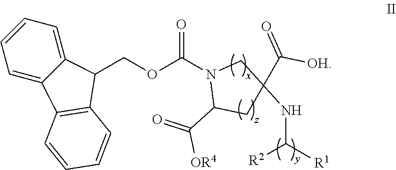

In the compounds of Formula II, y is an integer 0 or 1. In some embodiments, y is an integer 0. In other embodiments, y is an integer 1. In these compounds, x is an integer 1 or 2. In some embodiments, x is an integer 1. In other embodiments, x is an integer 2. Similarly, z is an integer 1 or 2. In some embodiments, z is an integer 1. In other embodiments, z is an integer 2. In further embodiments, both x and z are not 2.

R¹ is optionally substituted C₁₋₆ alkyl, optionally substituted C₂₋₆ alkenyl, optionally substituted C₂₋₆ alkynyl, or optionally substituted aryl. In some embodiments, R¹ is optionally substituted C₁₋₆ alkyl, such as methyl, ethyl, propyl, butyl, pentyl, or hexyl. In other embodiments, R¹ is optionally substituted C₂₋₆ alkenyl, such as ethenyl, propenyl, butenyl, pentenyl, or hexenyl. In further embodiments, R¹ is optionally substituted C₂₋₆ alkynyl, such as ethynyl, propynyl, butynyl, pentynyl, or hexynyl. In yet other embodiments, R¹ is optionally substituted aryl, such as phenyl. R² is absent, H, optionally substituted C₁₋₆ alkyl, optionally substituted C₂₋₆ alkenyl, optionally substituted C₂₋₆ alkynyl, or optionally substituted aryl. In some embodiments, R² is absent. In other embodiments, R² is H. In further embodiments, R² is optionally substituted C₁₋₆ alkyl, such as methyl, ethyl, propyl, butyl, pentyl, or hexyl. In other embodiments, R² is optionally substituted C₂₋₆ alkenyl, such as ethenyl, propenyl, butenyl, pentenyl, or hexenyl. In further embodiments, R² is optionally substituted C₂₋₆ alkynyl, such as ethynyl, propynyl, butynyl, pentynyl, or hexynyl. In yet other embodiments, R² is optionally substituted aryl, such as phenyl. R¹ and R² may be substituted with one or more groups. In some embodiments, R¹ and/or R² are, independently substituted with one, two, three, four, or five groups. In some embodiments, if R¹ and R² are substituted, the substituents may be the same or may differ.

In certain embodiments, R¹ and R² are, independently, substituted with C₀₋₆ alkylOH, C₀₋₆ alkylSH, C₀₋₆ alkylNH₂, C₀₋₆ alkyl-O—C₀₋₆ alkyl, C₀₋₆ alkyl-S—C₀₋₆ alkyl, C₀₋₆ alkylC(O)OH, C₀₋₆ alkylC(O)(C₁₋₆ alkyl), C₀₋₆ alkylC(O)O(C₁₋₆ alkyl), C₀₋₆ alkylN₃, C₀₋₆ alkylC(O)NH₂, C₀₋₆ alkyl-C(O)N(C₁₋₆ alkyl)OH, optionally substituted C₃₋₇cycloalkyl, optionally substituted C₅₋₇ cycloalkenyl, optionally substituted aryl, optionally substituted heteroaryl, optionally substituted heterocyclyl, —NHC(NH₂)(═N(C₀₋₆ alkyl), —NHC(NH₂)(═S(C₀₋₆ alkyl), a nucleobase, or an amino acid; wherein any carbon atom of said alkyl, alkenyl, or alkynyl is optionally replaced by a heteroatom that is O, S, SO, SO₂, or NR⁷. In other embodiments, R¹ and R² are, independently, substituted with C₀₋₆ alkylOH, such as OH or CH₂OH. In further embodiments, R¹ and R² are, independently, substituted with C₀₋₆ alkylSH, such as SH and CH₂SH. In yet other embodiments, R¹ and R² are, independently, substituted with C₀₋₆ alkylNH₂, such as NH₂, methanamine, ethanamine, propanamine, N,N-dimethylmethanamine, dimethylamine, or N,N,N-trimethylmethanamine. In still further embodiments, R¹ and R² are, independently, substituted with C₀₋₆ alkyl-O—C₀₋₆ alkyl, such as —O—C₁₋₆ alkyl, or OCH₃. In other embodiments, R¹ and R² are, independently, substituted with C₀₋₆ alkyl-S—C₀₋₆ alkyl, such as —S—C₁₋₆ alkyl, or such as —SCH₃. In further embodiments, R¹ and R² are, independently, substituted with C₀₋₆ alkylC(O)OH, such as C(O)OH, ethanoic acid, acetic acid, or propionic acid. In still other embodiments, R¹ and R² are, independently, substituted with C₀₋₆ alkylC(O)(C₁₋₆ alkyl), such as —C(O)(C₁₋₆ alkyl), or such as —CH₂—C(O)(C₁₋₆ alkyl), or such as —C(O)CH₃. In yet further embodiments, R¹ and R² are, independently, substituted with C₀₋₆ alkylC(O)O(C₁₋₆ alkyl), such as methyl formate or methyl acetate. In other embodiments, R¹ and R² are, independently, substituted with C₀₋₆ alkylN₃, such as N₃, or such as —CH₂N₃. In further embodiments, R¹ and R² are, independently, substituted with C₀₋₆ alkylC(O)NH₂, such as C(O)NH₂, or such as ethanoamide or propionamide. In still other embodiments, R¹ and R² are, independently, substituted with C₀₋₆ alkyl-C(O)N(C₁₋₆ alkyl)OH, such as —C(O)N(C₁₋₆ alkyl)OH, or such as —C(O)N(CH₃) OH, or such as carboxhydroxamide, ethanohydroxamide, or propionhydroxamide. In yet further embodiments, R¹ and R² are, independently, substituted with optionally substituted C₃₋₇cycloalkyl, such as cyclopropyl, cyclobutyl, cyclopentyl, cyclohexyl, or cycloheptyl, or such as cyclopropyl, or such as cyclobutyl. In other embodiments, R¹ and R² are, independently, substituted with optionally substituted C₅₋₇cycloalkenyl, such as cyclopentenyl, cyclohexenyl, or cycloheptenyl. In further embodiments, R¹ and R² are, independently, optionally substituted aryl, such as phenyl, p-cresol, 1-methoxy-benzy, naphthyl, 4-methyl-phenol, or 1-methoxy-4-methyl-benzene), or such as phenyl. In still other embodiments, R¹ and R² are, independently optionally substituted heteroaryl, such as imidazolyl, 2-pyrenyl, 1-methylimidazolyl, indolyl, pyridinyl such as 2-pyridinyl, 3-pyridinyl, or 4-pyridinyl, triazolyl, or imidazolyl. In yet further embodiments, R¹ and R² are, independently, optionally substituted heterocyclyl such as azetidinyl, dioxanyl, tetrahydropyranyl, pyrrolidinyl, piperidinyl, morpholinyl, piperazinyl, pyrrolidinyl, indolinyl, oxetanyl, tetrahydrofuranyl, tetrahydrothiophenyl, 2-pyrazolinyl, pyrazolidinyl, trithianyl, indolizinyl, benzo[b]thiophenyl, 1H-indazolyl, benzthiazolyl, 4 H-quinolizinyl, quinolinyl, 1,2,3,4-tetrahydroquinolinyl, isoquinolinyl, 1,2,3,4-tetrahydroisoquinolinyl, quinoxalinyl, phenothiazinyl. In other embodiments, R¹ and R² are, independently, —NHC(NH₂)(═N(C₀₋₆ alkyl), such as guanidinyl, methyl-guanidinyl, ethyl-guanidinyl, or propyl-guanidinyl. In further embodiments, R¹ and R² are, independently, —NHC(NH₂)(═S(C₀₋₆ alkyl), such as methyl-thiourea or ethyl-thiourea. In some embodiments, R¹ and R² may be protected with removable protective groups. For example, reactive nitrogens can be protected using Alloc groups.

In yet other embodiments, R¹ and R² are, independently, a nucleobase. Examples of such nucleobase include, but are not limited to, guaninyl, adeninyl, cytosinyl, thyminyl, or any combination thereof.

In still other embodiments, R¹ and R² are, independently, an amino acid, sugar, or any combination thereof. In some embodiments, the amino acid is a β-amino acid. Examples of amino acids include, but are not limited to, arginine (Arg), histidine (His), lysine (Lys), aspartate (Asp), glutamate (Glu), serine (Ser), threonine (Thr), asparagine (Asn), glutamine (Gln), cysteine (Cys), selecocysteine (Sec), glycine (Gly), proline (Pro), alanine (Ala), valine (Val), isoleucine (Ile), leucine (Leu), methionine (Met), phenylalanine (Phe), tyrosine (Tyr), tryptophan (Trp), pyrrolysine, or any combination thereof.

In further embodiments, R¹ and R² are linked together with the carbon they are attached to form a C₃₋₈ cycloalkyl such as a cyclopropyl, cyclobutyl, or cyclopentyl. In some embodiments, R¹ and R² are linked together to form a cyclopropyl. In other embodiments, R¹ and R² are linked together to form a cyclobutyl. In further embodiments, R¹ and R² are linked together to form a cyclopentyl. In yet other embodiments, R¹ and R² are linked together to form a cyclohexyl.

In certain embodiments, any carbon atom of the alkyl, alkenyl, or alkynyl group is optionally replaced by a O, S, SO, SO₂, or NR⁷. In some embodiments, any carbon atom of the alkyl, alkenyl, or alkynyl group is optionally replaced by an O heteroatom. In further embodiments, any carbon atom of the alkyl, alkenyl, or alkynyl group is optionally replaced by a S heteroatom. In yet other embodiments, any carbon atom of the alkyl, alkenyl, or alkynyl group is optionally replaced by a SO group. In still further embodiments, any carbon atom of the alkyl, alkenyl, or alkynyl group is optionally replaced by a SO₂ group. In other embodiments, any carbon atom of the alkyl, alkenyl, or alkynyl group is optionally replaced by a NR⁷, such as NH, or such as N(C₁₋₄ alkyl), or such as NCH₃.

R⁷ is H, C₁₋₄ alkyl, C₃₋₄ alkenyl, C₃₋₄ alkynyl, or C₁₋₄ bridging alkyl wherein a bridge is formed between the nitrogen and a carbon atom of said heteroatom-containing chain to form a ring, wherein said ring is optionally fused to Ar¹. In some embodiments, R⁷ is H. In other embodiments, R⁷ is C₁₋₄ alkyl, such as CH₃. In further embodiments, R⁷ is C₃₋₄ alkenyl. In yet other embodiments, R⁷ is C₃₋₄ alkynyl. In still further embodiments, R⁷ is a C₁₋₄ bridging alkyl wherein a bridge is formed between the nitrogen and a carbon atom of said heteroatom-containing chain to form a ring, wherein said ring is optionally fused to Ar¹.

Ar¹ is C₃₋₆ cycloalkyl, aryl, heterocyclyl, or heteroaryl. In some embodiments, Ar¹ is C₃₋₆ cycloalkyl, such as cyclopropyl, cyclobutyl, cyclopentyl, or cyclohexyl. In other embodiments, Ar¹ is aryl such as phenyl, 1-naphthyl, 2-naphthyl, indenyl, azulenyl, fluorenyl, or anthracyl. In further embodiments, Ar¹ is heterocyclyl such as azetidinyl, aziridinyl, piperidinyl, or azepanyl. In still other embodiments, Ar¹ is heteroaryl such as acridinyl, benzimidazolyl, benzthiazolyl, benzo[b]furanyl, benzo[b]thiophenyl, carbazolyl, cinnolinyl, furyl such as 2-furyl or 3-furyl, imidazolyl, 1H-indazolyl, indolizinyl, indolyl such as 3H-indolyl, indolinyl, isoindolyl, isoquinolinyl, isoxazolyl, 1,8-naphthyridinyl, oxadiazolyl such as 1,2,3-oxadiazolyl, oxazolyl, phenazinyl, phenothiazinyl, phenoxazinyl, pteridinyl, purinyl, pyrazinyl, pyrazolyl, 2-pyrazolinyl, pyrazolidinyl, pyridazinyl, pyrimidinyl, pyridyl such as 2-pyridyl, 3-pyridyl, or 4-pyridyl, pyrrolyl, 4 H-quinolizinyl, quinolinyl, phthalazinyl, quinazolinyl, quinoxalinyl, 1,2,3,4-tetrahydroquinolinyl, 1,2,3,4-tetrahydroisoquinolinyl, 1,3,4-thiadiazolyl, thiazolyl, 1,2,3-triazolyl, 1,3,5-triazinyl, 1,3,5-trithianyl, or thienyl such as 2-thienyl or 3-thienyl.

Ar¹ is optionally substituted with one or more of H, halo, OH, NO₂, —SO₃H, CF₃, OCF₃, C₁₋₆ alkyl such as CH₃, C₂₋₆ alkenyl, C₁₋₆ alkoxy such as OCH₃, O—C₃₋₄ alkenyl, —O-benzyl, —O-phenyl, 1,2-methylenedioxy, —NR⁵R⁶, —C(O)OH, —C(O)(C₁₋₆ alkyl), C(O)O(C₁₋₆ alkyl), -a carboxamide such as C(O)NH(C₁₋₆ alkyl), —C(O)NH(C₃₋₅alkenyl), —C(O)N(C₁₋₆ alkyl)(C₁₋₆ alkyl), —C(O)(C₃₋₅alkenyl)(C₃₋₆ alkenyl), morpholinyl, piperidinyl, —O—Ar², —CH₂—(CH₂)_{q}—Ar², —O—(CH₂)_{q}—Ar², —(CH₂)_{q}—O—Ar², or —CH—CH—Ar². R⁵ and R⁶ are, independently, H, C₁₋₆ alkyl, C₃₋₆ alkenyl, C₃₋₆ alkynyl, or benzyl. Ar² is 4-methoxyphenyl, 2-pyridyl, 3-pyridyl, 4-pyridyl, pyrazyl, quinolyl, 3,5-dimethylisoxazoyl, 2-methylthiazoyl, thiazolyl, 2-thienyl, 3-thienyl or pyrimidinyl. q is an integer 0 to 2.

In the compound of Formula II, X is a leaving group. One of skill in the art would readily be able to determine suitable leaving groups for use as “X.” In some embodiments, X is halo or a sulfonate. In other embodiments, X is halo, such as chloro, fluoro, or bromo. In further embodiments, X is a sulfonate such as p-toluenesulfonate (OTs), methanesulfonate (OMs), or trifluoromethanesulfonate (OTf). R⁴ in the compound of Formula II is a protecting group. In some embodiments, R⁴ is C₁₋₆ alkyl or 4-{N-[1-(4,4-dimethyl-2,6-dioxocyclohexylidene)-3-methylbutyl]amino}benzyl ester (DMab). In other embodiments, R⁴ is C₁₋₆ alkyl, such as t-butyl. In certain aspects, the compound of Formula II has the structure of Formula II-A.

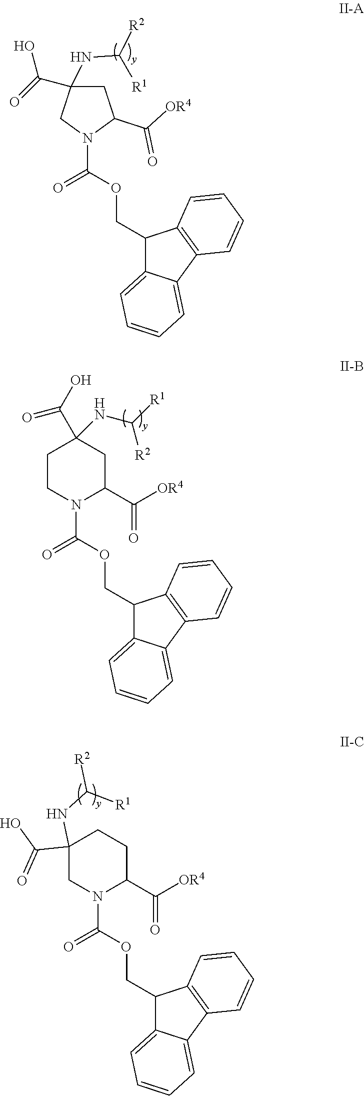

In further aspects, the compound of Formula II has the structure of Formula II-A. In other aspects, the compound of Formula II has the structure of Formula II-B. In further aspects, the compound of Formula II has the structure of Formula II-C.

The compounds of Formula II are prepared by contacting a compound of Formula I with R¹X, R¹C(O)H, or R¹C(O)R² in the presence of a reducing agent for a time and under conditions effective to produce a compound of Formula II:

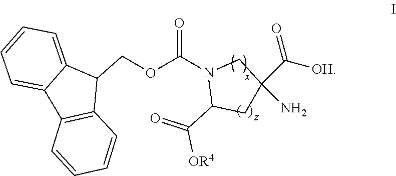

In some embodiments, when the compound of Formula I is reacted with R¹X, y is an integer 0 and R² is absent.

The organic solvent used to prepare the compounds of Formula II may be selected by those skill in the art. In some embodiments, the organic solvent is a polar organic solvent. In other embodiments, the organic solvent is an alcoholic solvent such as methanol; an ethereal solvent such as tetrahydrofuran, or ethyl acetate. The reducing agent used to form the compound of Formula II may be selected by one skilled in the art. In some embodiments, the reducing agent is NaBH₃, NaBH₃CN, or Na(CH₃COO)₃BH. In other embodiments, the reducing agent is NaBH₃CN.

In some aspects, the compound of Formula I has the structure of Formula I-A:

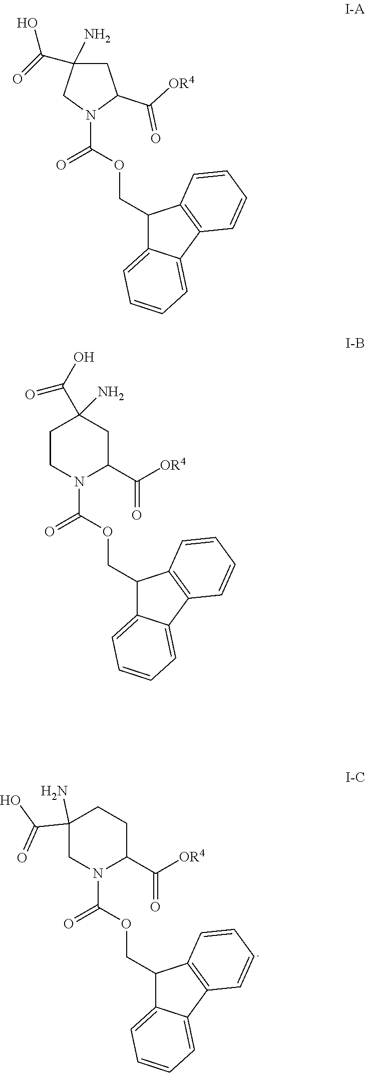

In further aspects, the compound of Formula I has the structure of Formula I-A. In other aspects, the compound of Formula I has the structure of Formula I-B. In further aspects, the compound of Formula I has the structure of Formula I-C.

The compounds of Formula I may be prepared by contacting a compound of Formula III with a chelator for a time and under conditions effective to produce the compound of Formula I:

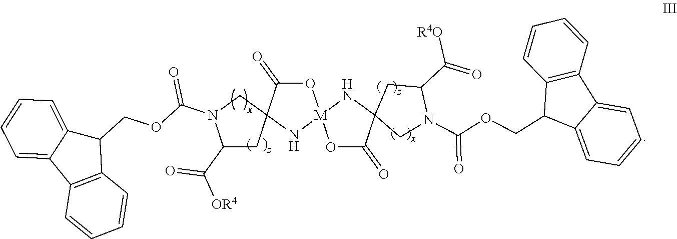

wherein, M is a transition metal with a +2 oxidation state. In some embodiments, M is vanadium, manganese, iron, cobalt, nickel, copper, or zinc. In other embodiments, M is copper. In further embodiments, M is manganese. In yet other embodiments, M is iron. In still further embodiments, M is cobalt. In other embodiments, M is nickel. In further embodiments, M is zinc.

The chelator used to prepare the compounds of Formula I may be selected by those skilled in the art. In some embodiments, the chelator is ethylenediaminetetraacetic acid (EDTA), diethylenetriaminepentaacetic acid (DTPA), or hydroxyethylethylenediaminetriacetic acid (HEDTA), or salts thereof. In further embodiments, the chelator is Na₂EDTA·H₂O or CaNa₂EDTA. In other embodiments, the chelator is H₂S, thiazolidinethione, glycine, and/or other chelators.

The organic solvent to prepare the compounds of Formula I may be selected by those skilled in the art. In some embodiments, the organic solvent is a polar organic solvent. In other embodiments, the organic solvent is an ethereal solvent, chlorinated organic solvent, or a combination thereof. In further embodiments, the organic solvent is an ethereal solvent such as methyl t-butyl ether. In still other embodiments, the organic solvent is a chlorinated organic solvent is chloroform. In yet further embodiments, the organic solvent is a mixture of methyl t-butyl ether and chloroform. When combinations/mixtures of solvents are utilized, the ratio is about 1:1 to about 1:5. In some embodiments, the ratio of methyl t-butyl ether to chloroform is about 1:1 to about 1:5.

In some aspects, the compound has the structure of Formula III-A, III-B, III-C, III-D, III-E, III-F, or III-G:

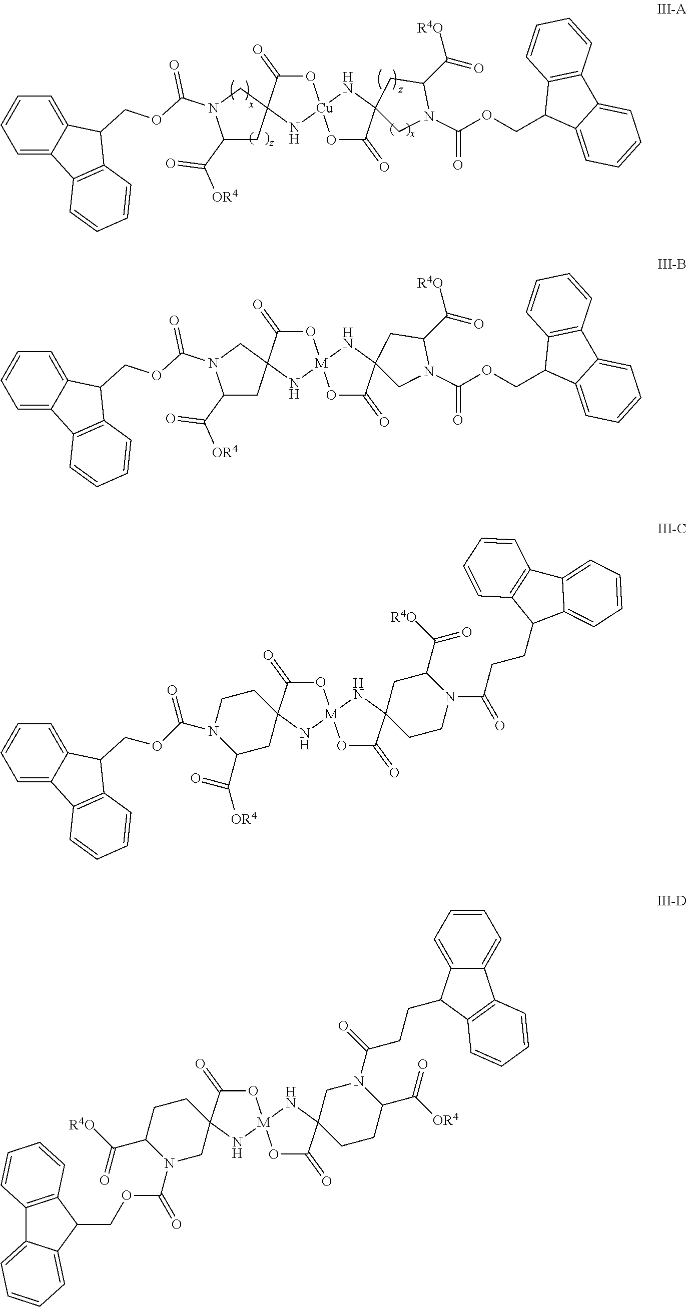

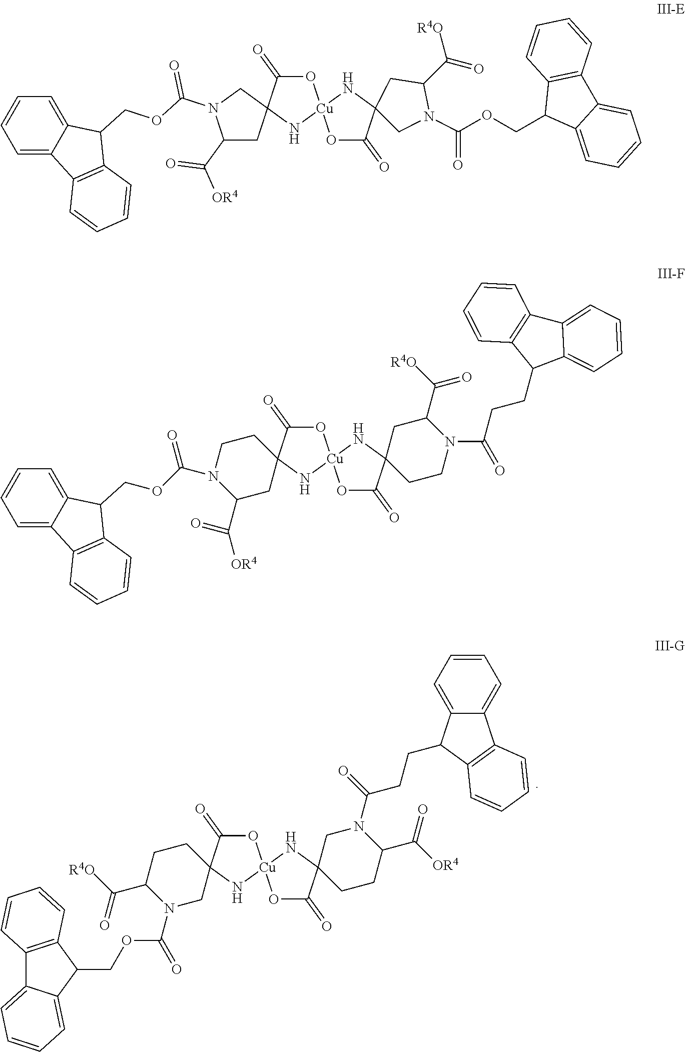

In further aspects, the compound of Formula III has the structure of Formula III-A. In other aspects, the compound of Formula III has the structure of Formula III-B. In further aspects, the compound of Formula III has the structure of Formula III-C. In yet other aspects, the compound of Formula III has the structure of Formula III-D. In still further aspects, the compound of Formula III has the structure of Formula III-E. In other aspects, the compound of Formula III has the structure of Formula III-F. In further aspects, the compound of Formula III has the structure of Formula or III-G.

The compounds of Formula III are prepared by contacting a compound of Formula IV:

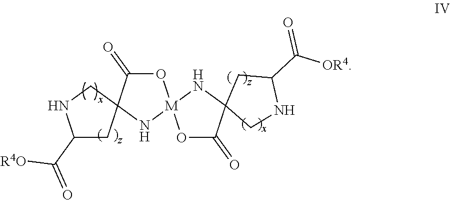

with 9-fluorenylmethoxycarbonyl chloride; for a time and under conditions effective to produce the compound of Formula IV. The organic solvent utilized to prepare the compound of Formula IV is a polar organic solvent. In some embodiments, the organic solvent is ethyl acetate or any solvents similar in polarity that enable the reaction to take place. In some embodiments, the organic solvent allows the reaction to take place. In some aspects, the compound of Formula IV has the structure of Formula IV-A, IV-B, IV-C, IV-D, IV-E, IV-F, or IV-G:

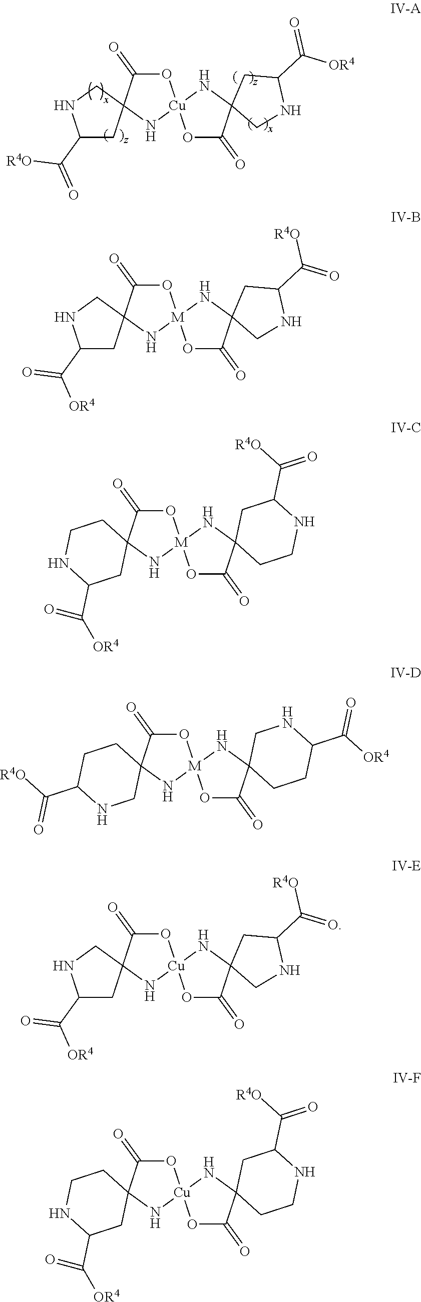

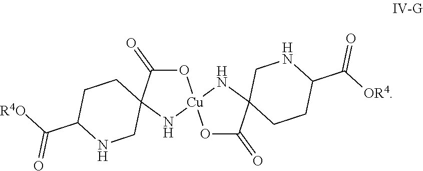

In further aspects, the compound of Formula IV has the structure of Formula IV-A. In other aspects, the compound of Formula IV has the structure of Formula IV-B. In further aspects, the compound of Formula IV has the structure of Formula IV-C. In yet other aspects, the compound of Formula IV has the structure of Formula IV-D. In still further aspects, the compound of Formula IV has the structure of Formula IV-E. In other aspects, the compound of Formula IV has the structure of Formula IV-F. In further aspects, the compound of Formula IV has the structure of Formula IV-G.

The compounds of Formula IV are prepared by contacting a compound of Formula V:

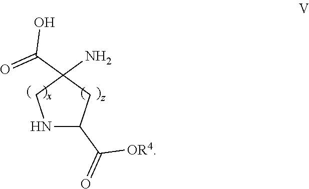

with a metal (II) source; for a time and under conditions effective to produce the compound of Formula IV. In some embodiments, the metal (II) source is a Ni (II) source or copper (II) source. In other embodiments, the metal source is a copper (II) source, such as a copper (II) salt. In further embodiments, the metal (II) source is a Ni (II) source. In still other embodiments, the copper (II) salt, such as copper chloride or copper sulfate. The solvent may be selected by those skilled in the art. In some embodiments, the solvent is water. In other embodiments, the solvent is optionally a mixture of water with one or more organic co-solvents.

In some aspects, the compound of Formula V has the structure of Formula V-A, V-B, or V-C:

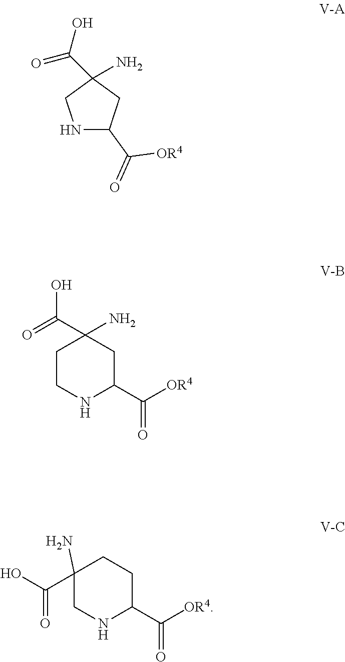

In further aspects, the compound of Formula V has the structure of Formula V-A. In other aspects, the compound of Formula V has the structure of Formula V-B. In further aspects, the compound of Formula V has the structure of Formula V-C.

The compound of Formula II may be contacted with a compound of Formula VI-A, VI-B, or VI-C, with an activating agent and a base to provide an intermediate:

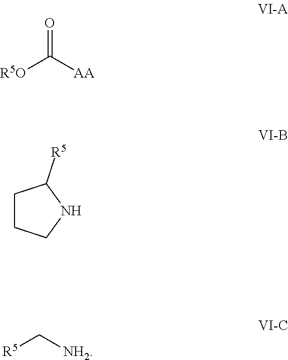

In certain embodiments, the compound of Formula II is contacted with a compound of Formula VI-A. In other embodiments, the compound of Formula II is contacted with a compound of Formula VI-B. In further embodiments, the compound of Formula II is contacted with a compound of Formula VI-C.

In some embodiments, AA is an optionally substituted amino acid. The amino acid may be selected by one skilled in the art. In some aspects, AA is a natural or non-natural amino acid. In certain aspects, AA is arginine (Arg), histidine (His), lysine (Lys), aspartate (Asp), glutamate (Glu), serine (Ser), threonine (Thr), asparagine (Asn), glutamine (Gln), cysteine (Cys), selecocysteine (Sec), glycine (Gly), proline (Pro), alanine (Ala), valine (Val), isoleucine (Ile), leucine (Leu), methionine (Met), phenylalanine (Phe), tyrosine (Tyr), tryptophan (Trp), or pyrrolysine. In certain aspects, AA is glycine. In other aspects, AA is alanine. In further aspects, AA is valine. In yet other aspects, AA is leucine. In still further aspects, AA is isoleucine. In other aspects, AA is proline. In further aspects, AA is serine. In yet other aspects, AA is threonine. In still further aspects, AA is asparagine. In other aspects, AA is glutamine. In further aspects, AA is cysteine. In still other aspects, AA is methionine. In yet further aspects, AA is phenylalanine. In other aspects, AA is tyrosine. In further aspects, AA is tryptophan. In yet other aspects, AA is aspartate. In still further aspects, AA is glutamate. In other aspects, AA is histidine. In further aspects, AA is lysine. In yet other aspects, AA is arginine. In still further aspects, AA is selecocysteine. In other aspects, AA is pyrrolysine. In further aspects, AA is a β-alanine.

AA may also be selected from among non-natural amino acids such as PABA, alloisoleucine, allothreonine, carboxyglutamic acid, cystathionine, D-alanine, dehydroalanine, D-glutamate, diaminopimelic acid, djenkolic acid, glycine betaine homocysteine, homonorleucine, homoserine, hydroxyglycine, hydroxyproline, hypusine, isoserine, isovaline, lanthionine, N-ethyl alanine, N-ethyl glycine, N-ethyl β-alanine, N-isopropyl glycine, N-methyl alanine, N-methyl β-alanine, norleucine, norvaline, N-propyl glycine, O-methyl-homoserine, ornithine, pipecolic acid, pyroglutamic acid, sarcosine, selenocysteine, selenohomocysteine, selenomethionine, selenoethionine, taurine, t-leucine, α,β-diaminopropionic acid, α,γ-diaminobutyric acid, α-aminoisobutyric acid, α-amino-n-butyric acid, α-amino-n-heptanoic acid, α-hydroxy-γ-aminobutyric acid, β-alanine, β-aminoisobutyric acid, β-amino-n-butyric acid, γ-aminobutyric acid, δ-aminolevulinic acid, 1-aminocyclopropane-1-carboxylic acid, azetidine-2-carboxylic acid, cycloleucine, or pseudoproline. In some aspects, AA is PABA. In other aspects, AA is alloisoleucine. In further aspects, AA is allothreonine. In yet other aspects, AA is carboxyglutamic acid. In still further aspects, AA is cystathionine. In other aspects, AA is D-alanine. In further aspects, AA is dehydroalanine. In yet other aspects, AA is D-glutamate. In still further aspects, AA is diaminopimelic acid. In yet further aspects, AA is djenkolic acid. In other aspects, AA is glycine betaine homocysteine. In further aspects, AA is homonorleucine. In yet other aspects, AA is homoserine. In still further aspects, AA is hydroxyglycine. In other aspects, AA is hydroxyproline. In further aspects, AA is hypusine. In still other aspects, AA is isoserine. In yet further aspects, AA is isovaline. In other aspects, AA is lanthionine. In further aspects, AA is N-ethyl alanine. In still other aspects, AA is N-ethyl glycine. In yet further aspects, AA is N-ethyl β-alanine. In other aspects, AA is N-isopropyl glycine. In further aspects, AA is N-methyl alanine. In still other aspects, AA is N-methyl β-alanine. In yet further aspects, AA is norleucine. In other aspects, AA is norvaline. In further aspects, AA is N-propyl glycine. In yet other aspects, AA is O-methyl-homoserine. In still further aspects, AA is ornithine. In other aspects, AA is pipecolic acid. In further aspects, AA is pyroglutamic acid. In yet other aspects, AA is sarcosine. In still further aspects, AA is selenocysteine. In other aspects, AA is selenohomocysteine. In further aspects, AA is selenomethionine. In yet other aspects, AA is selenoethionine. In still further aspects, AA is taurine. In other aspects, AA is t-leucine. In further aspects, AA is α,β-diaminopropionic acid. In yet other aspects, AA is α,γ-diaminobutyric acid. In still further aspects, AA is α-aminoisobutyric acid. In other aspects, AA is α-amino-n-butyric acid. In further aspects, AA is α-amino-n-heptanoic acid. In yet other aspects, AA is α-hydroxy-γ-aminobutyric acid. In still further aspects, AA is β-alanine. In other aspects, AA is β-aminoisobutyric acid. In further aspects, AA is β-amino-n-butyric acid. In yet other aspects, AA is γ-aminobutyric acid. In still further aspects, AA is δ-aminolevulinic acid. In other aspects, AA is 1-aminocyclopropane-1-carboxylic acid. In further aspects, AA is azetidine-2-carboxylic acid. In still other aspects, AA is cycloleucine. In yet further aspects, AA is pseudoproline. The amino acid attaches to the oxygen atom that was bound to the R⁴ group. The amino acid can bind to the oxygen atom through any position of the amino acid backbone. For example, in some embodiments, the amino acid attaches to the oxygen atom through a carbonyl of the amino acid.

In these structures, R⁵ is a resin. The resin suitable for use herein may be selected by one skilled in the art. In some embodiments, the resin contains a carboxy group. Desirably, the resin comprises a cleavable group. The term “cleavable group” as used herein refers to a group that is displaced from the compound of Formula V-C. In particular, the cleavable group is displaced from the compound of Formula V-C so that the AA can attach. In some embodiments, the cleavable group is OH or carboxyl. In other embodiments, the cleavable group is OH. In further embodiments, the cleavable group is carboxyl. In yet other embodiments, the cleavable group is a polystyrene resin.

The activating agent utilized may be selected by one of skill in the art.

In some aspects, the activating agent is 1-cyano-2-ethoxy-2-oxoethylidenaminooxy)dimethylamino-morpholino-carbenium hexafluorophosphate (COMU), 1H-1,2,3-benzotriazol-1-yloxy-tris(pyrrolidino)-phosphonium hexafluorophosphate (PyBOP), 1-hydroxy-7-azabenzotriazole (HOAt), 2-(1H-7-azabenzotriazol-1-yl)-1,1,3-tetramethyl uronium hexafluorophosphate (HATU), benzotriazol-1-yl-oxy-tris(dimethylamino)phosphonium hexafluorophosphate (BOP), chloro-N,N,N′,N′-tetramethylformamidinium hexafluorophosphate, dicyclohexylcarbodiimide (DCC), diisopropylcarbodiimide (DIC), ethyl cyano (hydroxyimino)acetato-O²]tri-1-pyrrolidinylphosphonium hexafluorophosphate (PyOxim), ethyl cyanohydroxyiminoacetate (Oxyma), N,N′-diisopropylcarbodiimide (DIC), N-hydroxybenzotriazole (HOBT), O-(1H-benzotriazol-1-yl)-N,N,N′,N′-tetramethyluronium tetrafluoroborate (TBTU), O-1,1,3,3-tetramethyluronium hexafluorophosphate (HCTU), or O-benzotriazole-N,N,N′,N′-tetramethyl uronium hexafluorophosphate (HBTU), or combinations thereof. In some aspects, the activating agent is COMU. In other aspects, the activating agent is PyBOP. In further aspects, the activating agent is HOAt. In yet other aspects, the activating agent is HATU. In still further aspects, the activating agent is BOP. In other aspects, the activating agent is chloro-N,N,N′,N′-tetramethylformamidinium hexafluorophosphate. In further aspects, the activating agent is DCC. In yet other aspects, the activating agent is DIC. In still further aspects, the activating agent is PyOxim. In other aspects, the activating agent is Oxyma. In further aspects, the activating agent is DIC. In still other aspects, the activating agent is HOBT. In yet further aspects, the activating agent is TBTU. In other aspects, the activating agent is HCTU. In further aspects, the activating agent is HBTU.

The solvent for this transformation may be selected by one skilled in the art. Preferably, the solvent is polar. Examples of solvents include NMP, DMF, DMSO, DCM, or mixtures thereof. In some aspects, the solvent is NMP. In other aspects, the solvent is DMF. In further aspects, the solvent is DMSO. In yet other aspects, the solvent is DCM.

The intermediate is then contacted with a deprotectant for a time and under conditions effective to produce the compound of Formula VII:

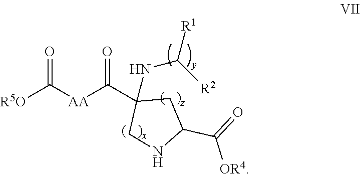

In some embodiments, the intermediate is converted to a compound of Formula VII-A, VII-B, or VII-C:

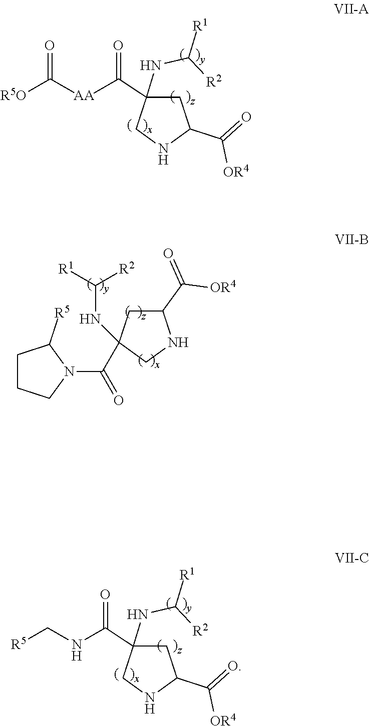

In some embodiments, the intermediate is converted to the compound of Formula VII-A. In other embodiments, the intermediate is converted to the compound of Formula VII-B. In further embodiments, the intermediate is converted to the compound of Formula VII-C.

One skilled in the art would be able to select a suitable deprotectant. In some embodiments, the deprotectant is a base. Examples of bases include, without limitation, piperidine, 4-methylpiperidine, piperazine, 1,8-diazabicyclo[5.4.0]undec-7-ene (DBU), or morpholine, or combinations thereof. In certain aspects, the base is piperidine. In other aspects, the base is 4-methylpiperidine. In further aspects, the base is piperazine. In yet other aspects, the base is DBU. In still further aspects, the base is morpholine.

The compound of Formula VII is then converted to the compound of Formula VIIIA, VIII-B, or VIII-C:

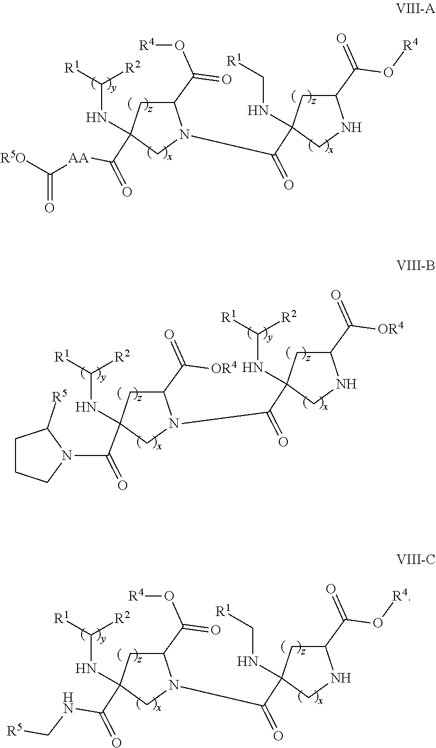

In some embodiments, the compound of Formula VII-A is converted to the compound of Formula VIII-A. In other embodiments, the compound of Formula VII-B is converted to the compound of Formula VIII-B. In further embodiments, the compound of Formula VII-C is converted to the compound of Formula VIII-C.

Such a conversion is performed by contacting the compound of Formula VII with the compound of Formula II in the presence of an activating agent as described above. Thereafter, the reaction is contacted with a deprotectant, as described above, for a time and under conditions effective to produce the compound of Formula VIII-A, VIII-B, or VIII-C.

Advantageously, the compound of Formula VIII-A, VIII-B, or VIII-C may be further reacted with the compound of Formula II to provide correspondingly larger compounds, such as those of Formula XV-A, XV-B, or XV-C:

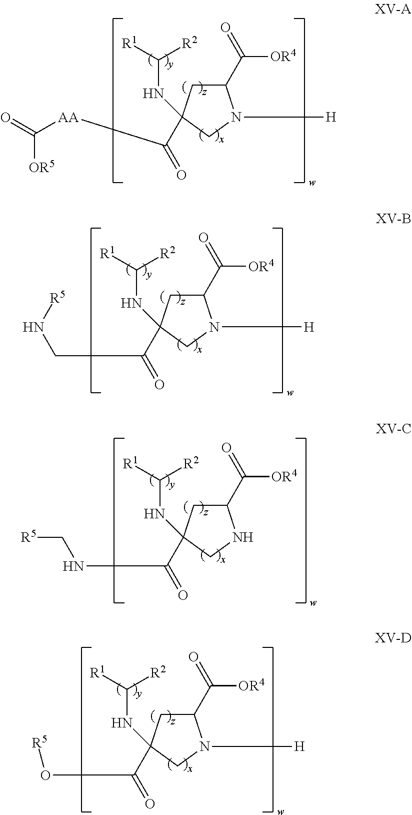

In other embodiments, the compounds of XV-A may be prepared. In further embodiments, compounds of XV-B may be prepared. In yet other embodiments, compounds of XV-C may be prepared.

As a general principle, these larger compounds are formed by reacting the product of each reaction involving a compound of Formula II with another compound of Formula II. The number of times (“w” times) that the reaction with compound of Formula II is performed is determined by the size of the compound that is desired. In certain embodiments, the product is reacted with the compound of Formula II one to twenty times (i.e., w is an integer from 1 to 20). In other embodiments, w is an integer 1, 2, 3, 4, 5, 6, 7, 8, 9, 10, 11, 12, 13, 14, 15, 16, 17, 18, 19, or 20. In further embodiments, w is an integer from 2 to 18, 2 to 16, 2 to 14, 2 to 12, 2 to 10, 2 to 8, 2 to 6, 2 to 4, 4 to 20, 4 to 18, 4 to 16, 4 to 14, 4 to 12, 4 to 10, 4 to 8, 4 to 6, 6 to 20, 6 to 18, 6 to 16, 6 to 14, 6 to 12, 6 to 10, 6 to 8, 8 to 20, 8 to 18, 8 to 16, 8 to 14, 8 to 12, 8 to 10, 10 to 20, 10 to 18, 10 to 16, 10 to 14, 10 to 12, 12 to 20, 12 to 18, 12 to 16, 12 to 14, 14 to 20, 14 to 18, 14 to 16, 16 to 20, 16 to 18, or 18 to 20. In yet other embodiments, w is an integer from 1 to 8. In still further embodiments, w is 6 to 8.

The compound of Formula VIII-A, VIII-B, or VIII-C may then be converted to the compound of Formula IX-B, IX-B, or IX-C:

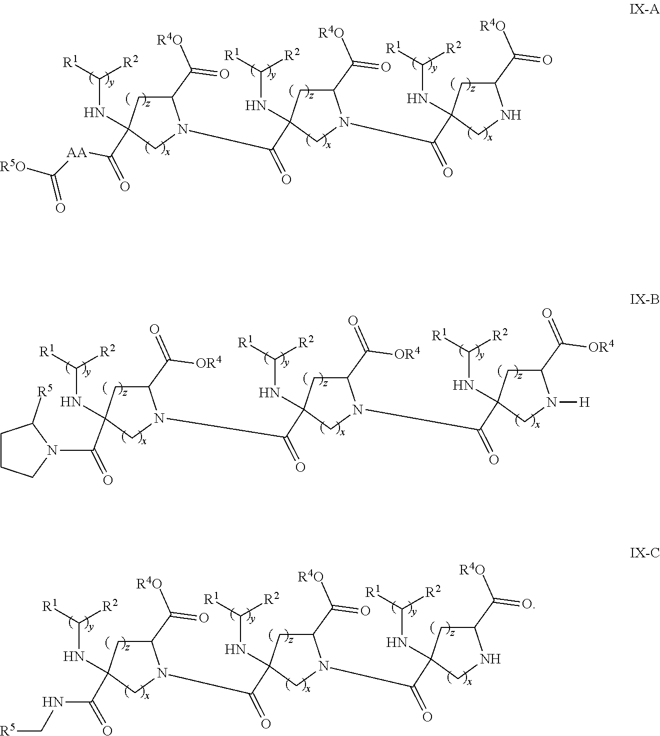

In some embodiments, the compound of Formula XIII-A is converted to the compound of Formula IX-A. In other embodiments, the compound of Formula VIII-B is converted to the compound of Formula IX-B. In further embodiments, the compound of Formula VIII-C is converted to the compound of Formula IX-C. Such a conversion is performed by contacting the compound of Formula VIII-A, VIII-B, or VIII-C with the compound of Formula II in the presence of an activating agent as described above. Thereafter, the reaction is contacted with a deprotectant, as described above, for a time and under conditions effective to produce the compound of Formula IX-A, IX-B, or IX-C.

The compound of Formula IX-A, IX-B, or IX-C may then be converted to a compound of Formula X-B, X-B, or X-C:

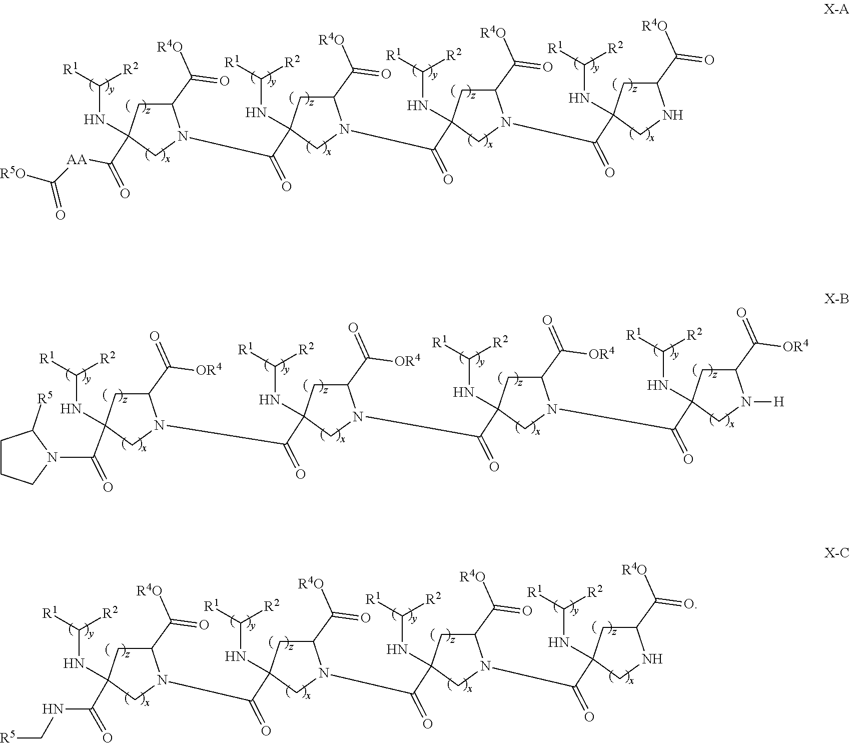

In some embodiments, the compound of Formula IX-A is converted to the compound of Formula X-A. In other embodiments, the compound of Formula XIII-A is converted to the compound of Formula X-B. In further embodiments, the compound of Formula XIII-A is converted to the compound of Formula X-C.

Such a conversion is performed by contacting the compound of Formula IX-A, IX-B, or IX-C with the compound of Formula II in the presence of an activating agent as described above. Thereafter, the reaction is contacted with a deprotectant, as described above, for a time and under conditions effective to produce the compound of Formula X-A, X-B, or X-C.

The compound of Formula X-A, X-B, or X-C may then be converted to the compound of Formula XI-B, XI-B, or XI-C:

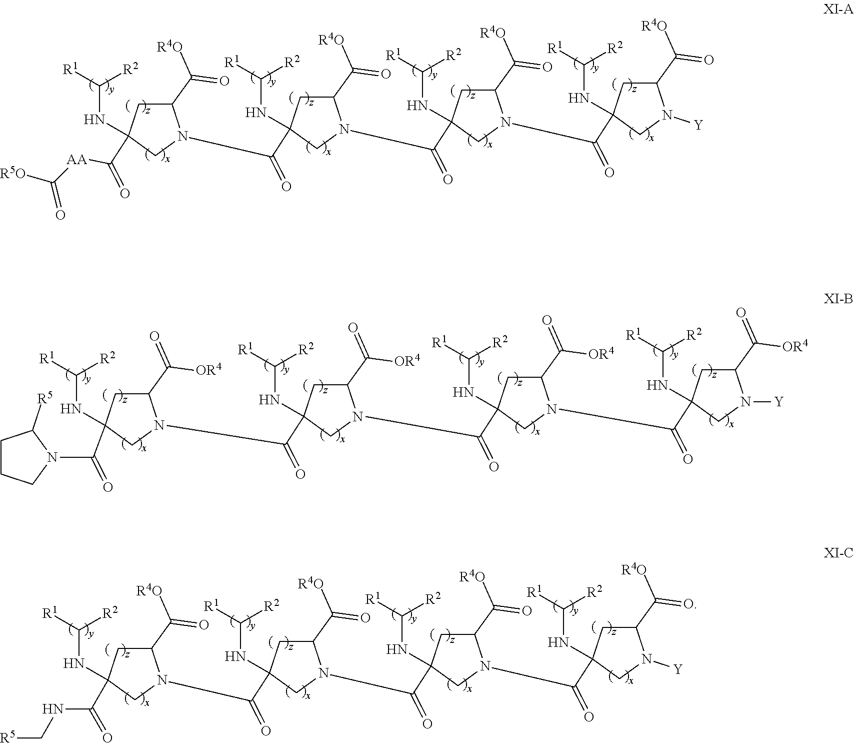

In some embodiments, the compound of Formula X-A is converted to the compound of Formula XI-A. In other embodiments, the compound of Formula X-B is converted to the compound of Formula XI-B. In further embodiments, the compound of Formula X-C is converted to the compound of Formula XI-C.

The compound of Formula X-A, X-B, or X-C is converted to the compound of Formula XI-A, XI-B, or XI-C by reaction with an activating agent as described herein and Y-Z. Y is defined as an aminocarbonyl group and Z is a leaving group. The term “aminocarbonyl group” as used herein refers to any chemical functional group that contains NH₂ and C(O) groups. Y is H or an amino acid. In some aspects, Y is H. In other aspects, Y is an amino acid. Examples of amino acids include those described herein. The amino acid may be selected by one skilled in the art. In some aspects, Y is a natural or non-natural amino acid. In certain aspects, Y is Arg, His, Lys, Asp, Glu, Ser, Thr, Asn, Gln, Cys, Sec, Gly, Pro, Ala, Val, Ile, Leu, Met, Phe, Tyr, Trp, or pyrrolysine. In certain aspects, Y is Gly. In other aspects, Y is Ala. In further aspects, Y is Val. In yet other aspects, Y is Leu. In still further aspects, Y is Ile. In other aspects, Y is Pro. In further aspects, Y is Ser. In yet other aspects, Y is Thr. In still further aspects, Y is Asp. In other aspects, Y is Gln. In further aspects, Y is Cys. In still other aspects, Y is Met. In yet further aspects, Y is Phe. In other aspects, Y is Tyr. In further aspects, Y is Trp. In yet other aspects, Y is Asp. In still further aspects, Y is Glu. In other aspects, Y is His. In further aspects, Y is Lys. In yet other aspects, Y is Arg. In still further aspects, Y is selecocysteine. In other aspects, Y is pyrrolysine In further aspects, Y is a β-alanine.

Y may also be selected from among non-natural amino acids such as PABA, alloisoleucine, allothreonine, carboxyglutamic acid, cystathionine, D-alanine, dehydroalanine, D-glutamate, diaminopimelic acid, djenkolic acid, glycine betaine homocysteine, homonorleucine, homoserine, hydroxyglycine, hydroxyproline, hypusine, isoserine, isovaline, lanthionine, N-ethyl alanine, N-ethyl glycine, N-ethyl β-alanine, N-isopropyl glycine, N-methyl alanine, N-methyl β-alanine, norleucine, norvaline, N-propyl glycine, O-methyl-homoserine, ornithine, pipecolic acid, pyroglutamic acid, sarcosine, selenocysteine, selenohomocysteine, selenomethionine, selenoethionine, taurine, t-leucine, α,β-diaminopropionic acid, α,γ-diaminobutyric acid, α-aminoisobutyric acid, α-amino-n-butyric acid, α-amino-n-heptanoic acid, α-hydroxy-γ-aminobutyric acid, β-alanine, β-aminoisobutyric acid, β-amino-n-butyric acid, γ-aminobutyric acid, δ-aminolevulinic acid, 1-aminocyclopropane-1-carboxylic acid, azetidine-2-carboxylic acid, cycloleucine, or pseudoproline. In some aspects, Y is PABA. In other aspects, Y is alloisoleucine. In further aspects, Y is allothreonine. In yet other aspects, Y is carboxyglutamic acid. In still further aspects, Y is cystathionine. In other aspects, Y is D-alanine. In further aspects, Y is dehydroalanine. In yet other aspects, Y is D-glutamate. In still further aspects, Y is diaminopimelic acid. In yet further aspects, Y is djenkolic acid. In other aspects, Y is glycine betaine homocysteine. In further aspects, Y is homonorleucine. In yet other aspects, Y is homoserine. In still further aspects, Y is hydroxyglycine. In other aspects, Y is hydroxyproline. In further aspects, Y is hypusine. In still other aspects, Y is isoserine. In yet further aspects, Y is isovaline. In other aspects, Y is lanthionine. In further aspects, Y is N-ethyl alanine. In still other aspects, Y is N-ethyl glycine. In yet further aspects, Y is N-ethyl β-alanine. In other aspects, Y is N-isopropyl glycine. In further aspects, Y is N-methyl alanine. In still other aspects, Y is N-methyl β-alanine. In yet further aspects, Y is norleucine. In other aspects, Y is norvaline. In further aspects, Y is N-propyl glycine. In yet other aspects, Y is O-methyl-homoserine. In still further aspects, Y is ornithine. In other aspects, Y is pipecolic acid. In further aspects, Y is pyroglutamic acid. In yet other aspects, Y is sarcosine. In still further aspects, Y is selenocysteine. In other aspects, Y is selenohomocysteine. In further aspects, Y is selenomethionine. In yet other aspects, Y is selenoethionine. In still further aspects, Y is taurine. In other aspects, Y is t-leucine. In further aspects, Y is α,β-diaminopropionic acid. In yet other aspects, Y is α,γ-diaminobutyric acid. In still further aspects, Y is α-aminoisobutyric acid. In other aspects, Y is α-amino-n-butyric acid. In further aspects, Y is α-amino-n-heptanoic acid. In yet other aspects, Y is α-hydroxy-γ-aminobutyric acid. In still further aspects, Y is β-alanine. In other aspects, Y is β-aminoisobutyric acid. In further aspects, Y is β-amino-n-butyric acid. In yet other aspects, Y is γ-aminobutyric acid. In still further aspects, Y is δ-aminolevulinic acid. In other aspects, Y is 1-aminocyclopropane-1-carboxylic acid. In further aspects, Y is azetidine-2-carboxylic acid. In still other aspects, Y is cycloleucine. In yet further aspects, Y is pseudoproline.

Thereafter, a deprotectant, as described above, is added for a time and under conditions effective to produce the compound of Formula XI-A, XI-B, or XI-C, respectively.

Z is a leaving group, which may be selected by those skilled in the art. In some embodiments, Z is a halo, sulfonate, N-hydroxybenzotriazolyl, 1-hydroxy-7-azabenzotriazolyl, or

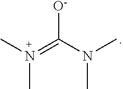

In other embodiments, Z is a halo such as chloro, fluoro, or bromo. In further embodiments, Z is a sulfonate such as p-toluenesulfonate (OTs), methanesulfonate (OMs), or trifluoromethanesulfonate (OTf). In yet other embodiments, Z is N-hydroxybenzotriazolyl. In still further embodiments, Z is 1-hydroxy-7-azabenzotriazolyl. In other embodiments, Z is

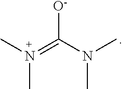

The compound of Formula XI-A may then be contacted with a weak acid for a time and under conditions sufficient to produce a compound of Formula XII-A:

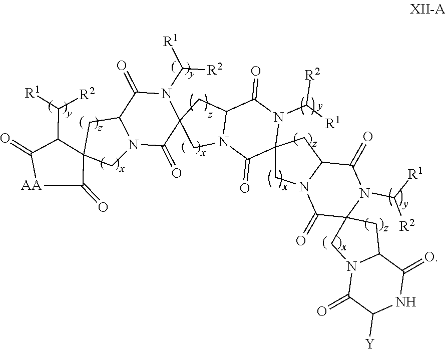

In some embodiments, a compound of Formula XII-A is produced.

The weak acid utilized in the transformation may be selected by one of skill in the art. In some embodiments, the weak acid is trifluoroacetic acid or acetic acid. In other embodiments, the weak acid is trifluoracetic acid. In further embodiments, the weak acid is acetic acid.

In some embodiments, the weak acid has a pH between about 1 to about 7. In some embodiments, the weak acid has a pH between about 2 to about 6. In some embodiments, the weak acid has a pH between about 2 to about 5. In some embodiments, the weak acid has a pH between about 3 to about 4. In one embodiment, the weak acid has a pH of about 7. In one embodiment, the weak acid has a pH of about 6. In one embodiment, the weak acid has a pH of about 5. In one embodiment, the weak acid has a pH of about 4. In one embodiment, the weak acid has a pH of about 3. In one embodiment, the weak acid has a pH of about 2. In one embodiment, the weak acid has a pH of about 1.

In some embodiments, the weak acid has a concentration of about 50 to about 200 mM. In still further embodiments, the weak acid has a concentration of about 50, about 75, about 100, about 125, about 150, about 175, or about 200 mM. In other embodiments, the weak acid has a concentration of about 50 to about 175, about 50 to about 150, about 50 to about 125, about 50 to about 100, about 50 to about 75, about 75 to about 200, about 75 to about 175, about 75 to about 150, about 75 to about 125, about 75 to about 100, about 100 to about 200, about 100 to about 175, about 100 to about 150, about 100 to about 125, about 125 to about 200, about 125 to about 175, about 125 to about 150, about 150 to about 200, about 150 to about 175, or about 175 to about 200 mM. In further embodiments, the weak acid is about 50 to about 200 mM acetic acid.

The weak acid may contain a solvent. Examples of such solvents, but are not limited to, methanol, ethanol, isopropanol, and/or butyl alcohol, or the like, and/or polar aprotic solvents, such as dimethylformamide, N-methylpyrrolidinone, etc.

The amount of time required to prepare the spirocyclic compounds described herein, including the compounds of Formula XII-A, is about 1 minute to about 24 hours, preferably about 2 to about 12 hours. In some embodiments, the time is about 1 minute, about 5 minutes, about 15 minutes, about 30 minutes, about 1 hour, about 2 hours, about 3 hours, about 4 hours, about 5 hours, 6 hours, 7 hours, 8 hours, 9 hours, 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In other embodiments, the time is about 1 minute to about 18 hours, about 1 minute to about 12 hours, about 1 minute to about 8 hours, about 1 minute to about 4 hours, about 1 minute to about 1 hour, about 1 minute to about 30 minutes, about 30 minutes to about 24 hours, about 30 minutes to about 18 hours, about 30 minutes to about 12 hours, about 30 minutes to about 8 hours, about 30 minutes to about 4 hours, about 30 minutes to about 1 hour, about 1 hour to about 24 hours, about 1 hour to about 18 hours, about 1 hour to about 12 hours, about 1 hour to about 8 hours, about 1 hour to about 4 hours, about 4 hours to about 24 hours, about 4 hours to about 18 hours, about 4 hours to about 12 hours, about 4 hours to about 8 hours, about 8 hours to about 24 hours, about 8 hours to about 18 hours, about 8 hours to about 12 hours, about 12 hours to about 24 hours, about 12 hours to about 18 hours, or about 18 to about 24 hours.

The temperature utilized to prepare the compounds of Formula XII-A, is about room temperature to an elevated temperature. The term “room temperature” as used herein refers to a temperature of about 30 to about 35° C. In some embodiments, the temperature is about 30 to about 60° C. In other embodiments, the temperature is about 30, about 35, about 40, about 45, about 50, about 55, or about 60° C. In further embodiments, the temperature is about 30 to about 55, about 30 to about 50, about 30 to about 45, about 30 to about 40, about 30 to about 35, about 35 to about 60, about 35 to about 55, about 35 to about 50, about 35 to about 45, about 35 to about 40, about 40 to about 60, about 40 to about 55, about 40 to about 50, about 40 to about 45, about 45 to about 60, about 45 to about 55, about 45 to about 50, about 50 to about 60, about 50 to about 55, or about 55 to about 60° C.

The present invention further provides processes for preparing compounds of Formula XV:

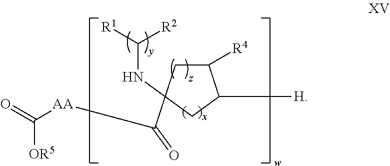

In the structure of Formula XV, R¹, R², R⁴, x, and are defined herein. w is an integer from 1 to 20. In some embodiments, w is an integer 1, 2, 3, 4, 5, 6, 7, 8, 9, 10, 11, 12, 13, 14, 15, 16, 17, 18, 19, or 20. In other embodiments, w is an integer from 1 to 18, 1 to 16, 1 to 14, 1 to 12, 1 to 10, 1 to 8, 1 to 6, 1 to 4, 1 to 2, 2 to 20, 2 to 18, 2 to 16, 2 to 14, 2 to 12, 2 to 10, 2 to 8, 2 to 6, 2 to 4, 4 to 20, 4 to 18, 4 to 16, 4 to 14, 4 to 12, 4 to 10, 4 to 8, 4 to 6, 6 to 20, 6 to 18, 6 to 16, 6 to 14, 6 to 12, 6 to 10, 6 to 8, 8 to 20, 8 to 18, 8 to 16, 8 to 14, 8 to 12, 8 to 10, 10 to 20, 10 to 18, 10 to 16, 10 to 14, 10 to 12, 12 to 20, 12 to 18, 12 to 16, 12 to 14, 14 to 20, 14 to 18, 14 to 16, 16 to 20, 16 to 18, or 18 to 20. In the structure of the compound of Formula XV, y is an integer 0 or 1. In certain embodiments, y is an integer 0. In other embodiments y is an integer 1.

The processes for preparing the compound of Formula XV include compound of Formula II with a compound of Formula VI, wherein x, z, R¹, R², R⁵, and AA are defined herein:

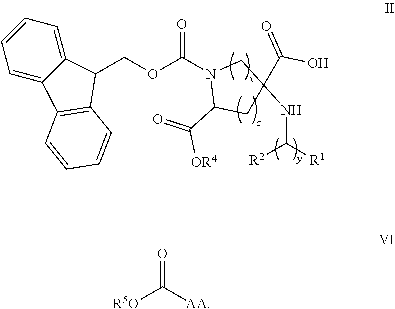

The processes are performed with an activating agent, as defined herein, and a base, as defined herein, to provide an intermediate. The intermediate is then contacted with a deprotectant, as defined herein, for a time and under conditions effective to produce the compound of Formula VII:

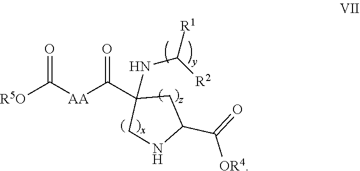

The compound of Formula VII is then contacted with the compound of Formula II (w-1) times, wherein w is defined herein. The term “w-1” times as used herein means that the compound of Formula VII is contacted with the compound of Formula II to provide a first intermediate. That first intermediate is then contacted with the compound of Formula II to provide a second intermediate, etc. In some embodiments, the compound of Formula VII is contacted with the compound of Formula II 1-19 times In some embodiments, the compound of Formula VII is contacted with the compound of Formula II 1, 2, 3, 4, 5, 6, 7, 8 9, 10, 11, 12, 13, 14, 15, 16, 17, 18 or 19 times. In other embodiments, the compound of Formula VII is contacted with the compound of Formula II w is an integer from 1 to 18, 1 to 16, 1 to 14, 1 to 12, 1 to 10, 1 to 8, 1 to 6, 1 to 4, 1 to 2, 2 to 20, 2 to 18, 2 to 16, 2 to 14, 2 to 12, 2 to 10, 2 to 8, 2 to 6, 2 to 4, 4 to 20, 4 to 18, 4 to 16, 4 to 14, 4 to 12, 4 to 10, 4 to 8, 4 to 6, 6 to 20, 6 to 18, 6 to 16, 6 to 14, 6 to 12, 6 to 10, 6 to 8, 8 to 20, 8 to 18, 8 to 16, 8 to 14, 8 to 12, 8 to 10, 10 to 20, 10 to 18, 10 to 16, 10 to 14, 10 to 12, 12 to 20, 12 to 18, 12 to 16, 12 to 14, 14 to 20, 14 to 18, 14 to 16, 16 to 20, 16 to 18, or 18 to 20 times.

The present invention also provides process for preparing compounds of Formula I, wherein x, z, and R⁴ are defined herein:

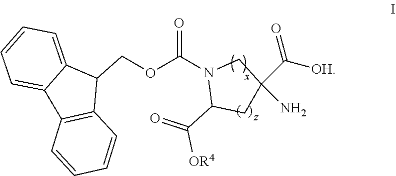

The processes include contacting a compound of Formula III, wherein, M is a transition metal with a +2 oxidation state as defined herein:

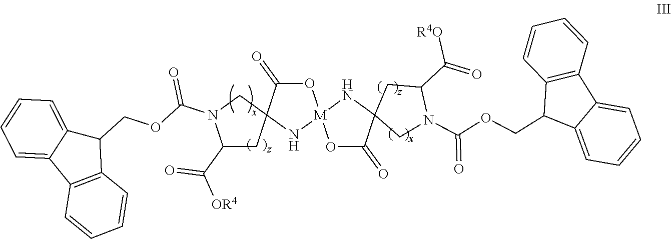

The processes are performed with a chelator, as defined herein, for a time and under conditions effective to produce the compound of Formula I.

The present invention further provides processes for preparing compounds of Formula III, wherein X, Z, and R⁴ are defined herein:

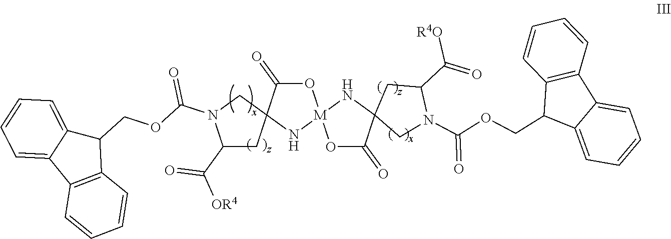

The processes include contacting a compound of Formula IV with 9-fluorenylmethoxycarbonyl chloride for a time and under conditions effective to produce the compound of Formula III.

The present invention further provides processes for preparing compounds of Formula IV, wherein x, z, and R⁴ are defined herein:

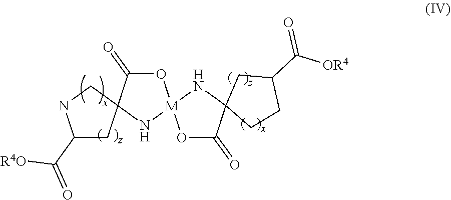

The processes include contacting a compound of Formula V with a metal (II) source, as defined herein:

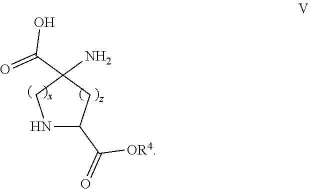

The processes are performed for a time and under conditions effective to produce the compound of Formula IV.

The present invention also provides processes for preparing compounds of Formula VII, wherein x, y, z, R⁴, R⁵, and AA are defined herein:

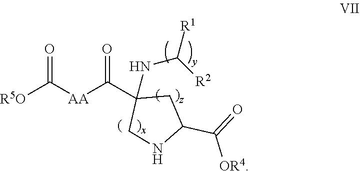

The processes include contacting a compound of Formula II:

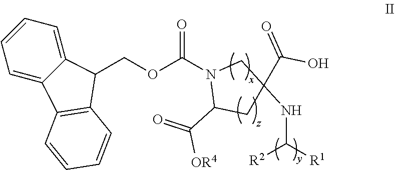

with a compound of Formula VI:

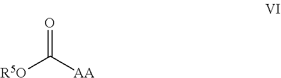

The processes are performed using an activating agent, as defined herein, and a base, as defined herein, to provide an intermediate; and contacting the intermediate with a deprotectant, as defined herein, for a time and under conditions effective to produce the compound of Formula VII.

### The Compounds

Advantageously, the processes described herein permit the preparation of an unlimited numbers of compounds. In certain embodiments, the present invention provides compounds of Formula XIV:

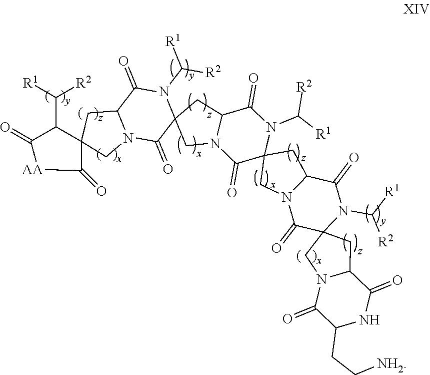

In this structure, each instance of R¹, R², X, y, and z is, independently, defined herein.

Also provided are compounds of Formula I, or a salt thereof, wherein y, R¹, R², and R⁴ are defined herein:

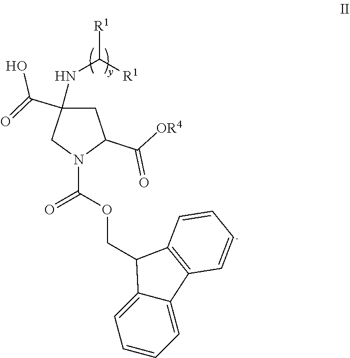

The present invention also provides compounds of Formula II-A, II-B, or II-C, wherein R¹ and R⁴ are defined herein:

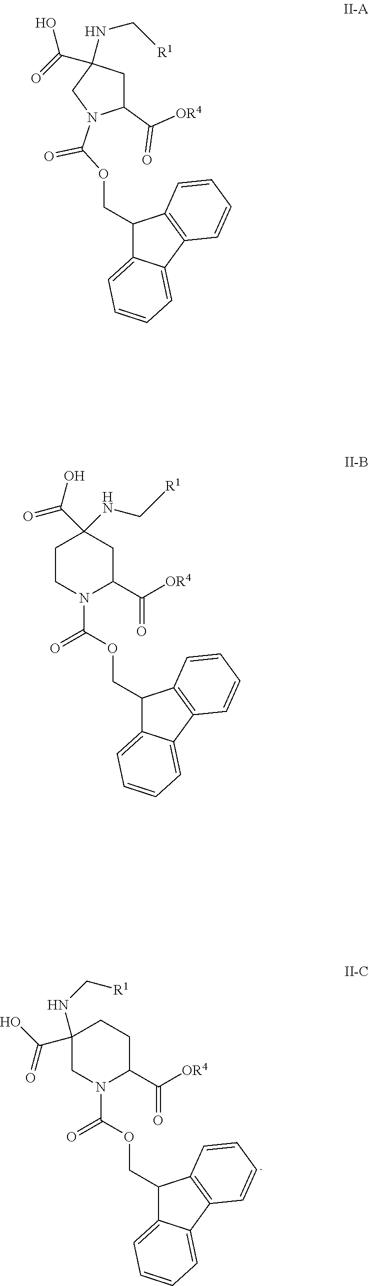

In some embodiments, the compound is of Formula II-A. In other embodiments, the compound is of Formula II-B. In further embodiments, the compound is of Formula II-C.

In yet other embodiments, the compound is:

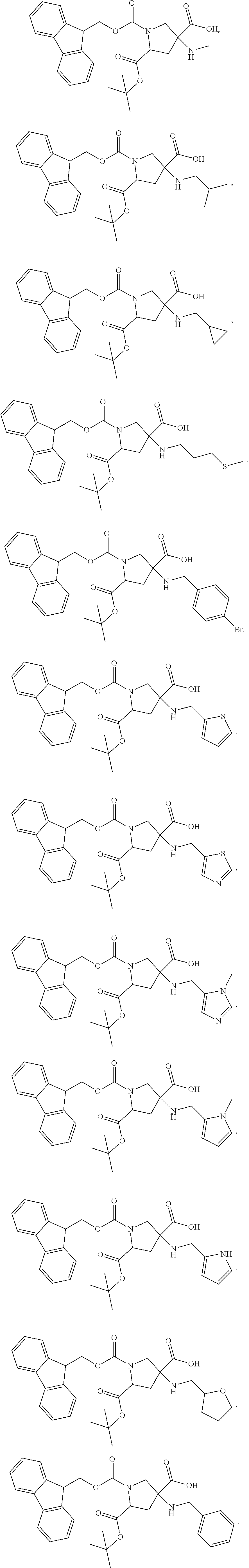

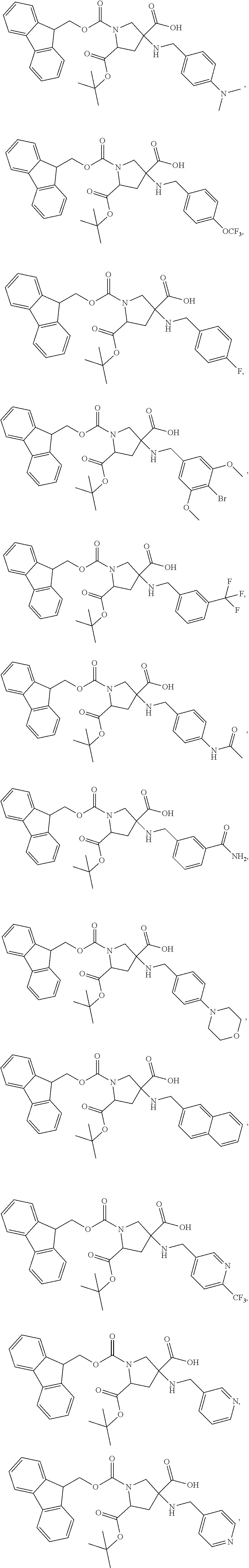

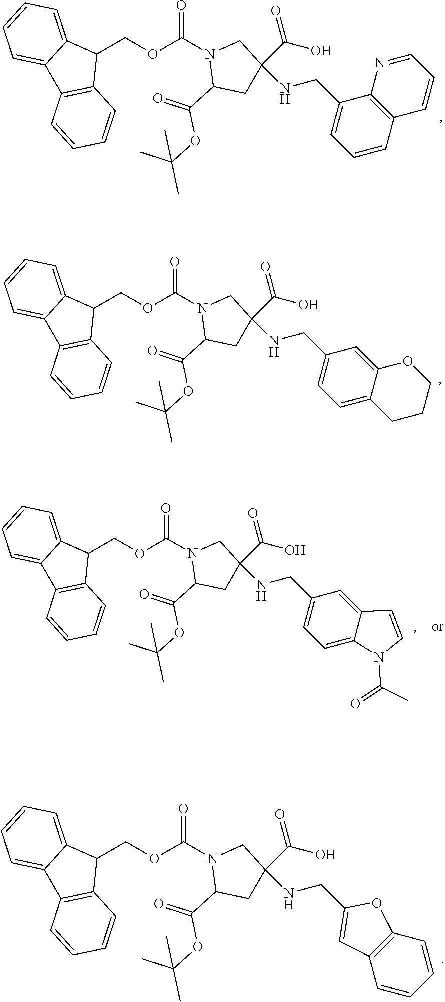 [SMILES: CC(C)(C)OC(=O)C1CC(NCc2cccc3cccnc23)(C(=O)O)CN1C(=O)OCC1c2ccccc2-c2ccccc21 ; CC(C)(C)OC(=O)C1CC(NCc2ccc3c(c2)OCCC3)(C(=O)O)CN1C(=O)OCC1c2ccccc2-c2ccccc21 ; CC(=O)n1ccc2cc(CNC3(C(=O)O)CC(C(=O)OC(C)(C)C)N(C(=O)OCC4c5ccccc5-c5ccccc54)C3)ccc21 ; CC(C)(C)OC(=O)C1CC(NCc2cc3ccccc3o2)(C(=O)O)CN1C(=O)OCC1c2ccccc2-c2ccccc21]

In still further compounds, the present invention provides a compound that is:

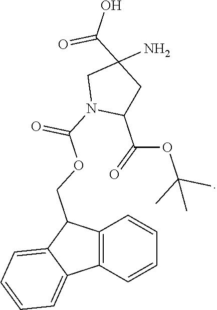 [SMILES: CC(C)(C)OC(=O)C1CC(N)(C(=O)O)CN1C(=O)OCC1c2ccccc2-c2ccccc21]

The present invention further provides a compound that is:

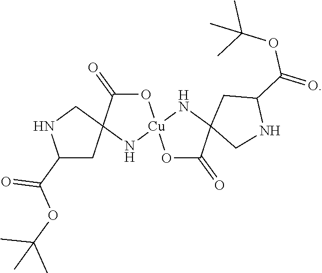

The present invention also provides a compound that is:

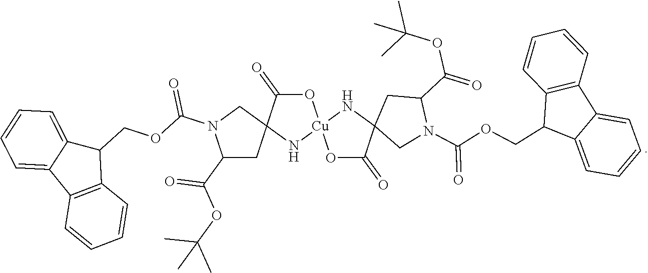

It is to be understood that while the present invention has been described in conjunction with the preferred specific embodiments thereof, that the foregoing description and the examples that follow are intended to illustrate and not limit the scope of the present invention. It will be understood by those skilled in the art that various changes may be made and equivalents may be substituted without departing from the scope of the invention, and further that other aspects, advantages and modifications will be apparent to those skilled in the art to which the present invention pertains. In addition to the embodiments described herein, the present invention contemplates and claims those inventions resulting from the combination of features of the present invention cited herein and those of the cited prior art references which complement the features of the present invention.

The present inventions of each patent, patent application, and publication cited or described in this document are hereby incorporated herein by reference, each in its entirety, for all purposes.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: The Development of Fmoc-Protected Bis-Amino Acids Towards Automated Synthesis of Highly Functionalized Spiroligomers

“Molecular structure defines function”—this is the most fundamental paradigm of molecular biology (Gutteridge, A. et al., Trends in Biochemical Sciences 2005, 30 (11), 622-629). It is a goal of macromolecular chemistry to create ever-larger molecules with control over their three-dimensional structure and the constellation of functional groups that they present (Lenci, E. et al., Chemical Society Reviews 2020, 49 (11), 3262-3277; Lutz, J.-F et al., Science 2013, 341 (6146), 1238149). Stoddart first introduced the concept of “molecular LEGO”, which can be programmed to have desired shapes through iterative ring fusion, such as the belt[n] arenes and kohnkenes (Hill, D. J. et al., Chemical Reviews 2001, 101 (12), 3893-4012). More recently, the Bode group has demonstrated the iterative assembly of polycyclic saturated heterocycles from monomeric building blocks (Saito, F et al., Journal of the American Chemical Society 2019, 141 (13), 5544-5554).

Spiroligomers are fused-ring spiro-ladder structures constructed from cyclic, stereochemically pure bis-amino acid building blocks joined together through diketopiperazine (DKP) rings (Schafmeister, C. E. et al., Accounts of Chemical Research 2008, 41 (10), 1387-1398). The formation of spirocyclic DKPs enforces the rigidity of the backbone by eliminating single bond rotation in the backbone. Meanwhile, the positions and orientations of various functional groups on the backbones are dictated by the sequence and stereochemistry of building blocks. Various applications of spiroligomers are being developed, including as catalysts of organic reactions (Parker, M. F. L. et al., Journal of the American Chemical Society 2014, 136 (10), 3817-3827), templates of supramolecular metal-binding complexes (Northrup, J. D. et al., Journal of Organic Chemistry 2021, 86 (6), 4867-4876), the inhibitors of protein-protein interactions (Brown, Z. Z et al., Plos One 2012, 7 (10)) and carbohydrate binding molecules (Chepyshev, S. V. et al., Rocky Mountain Regional Meeting, Fort Collins, CO, United States 2020).

Functionalized spiroligomer synthesis has been difficult because the monomer syntheses are time-consuming even at a scale of ˜600 mmol. Previously, the original carboxybenzyl (Cbz), the tert-butoxycarbonyl (Boc) and the p-nitrobenzyloxycarbonyl (pNZ) have been used as the chain-extension removable protecting group for the proline amine in solid phase synthesis. These protecting groups were either difficult to remove such as Cbz, or they led to side-reactions during deprotection and limited the choices of resin linking groups to those that provide low yields (Cheong, J. E., Tetrahedron Letters 2016, 57 (44), 4882-4884; Pfeiffer, C. T. et al, Tetrahedron Letters 2018, 59 (30), 2884-2888).

The Fluorenylmethyloxycarbonyl (Fmoc) group, as an excellent temporary protecting group in peptide synthesis, allowed the use of excellent high-yielding cleavable resin linkers, such as the chloro-trityl linker (Behrendt, R. et al., Journal of Peptide Science 2016, 22 (1), 4-27; Ieronymaki, M. et al., Biopolymers 2015, 104 (5), 506-514; Stathopoulos, P. et al., Journal of Peptide Science 2006, 12 (3), 227-232). The herein described studies efficiently incorporated the Fmoc group into spiroligomer synthesis. The approach combined a scaled-up synthesis of bis-amino acid intermediates at tens of kilograms scale and an efficient one-pot synthetic methodology to replace the Cbz group with the Fmoc group. Four unique spiroligomers were synthesized using the Fmoc/tBu solid phase synthesis, and their three-dimensional structures were confirmed by nuclear magnetic resonance (NMR) spectroscopy.

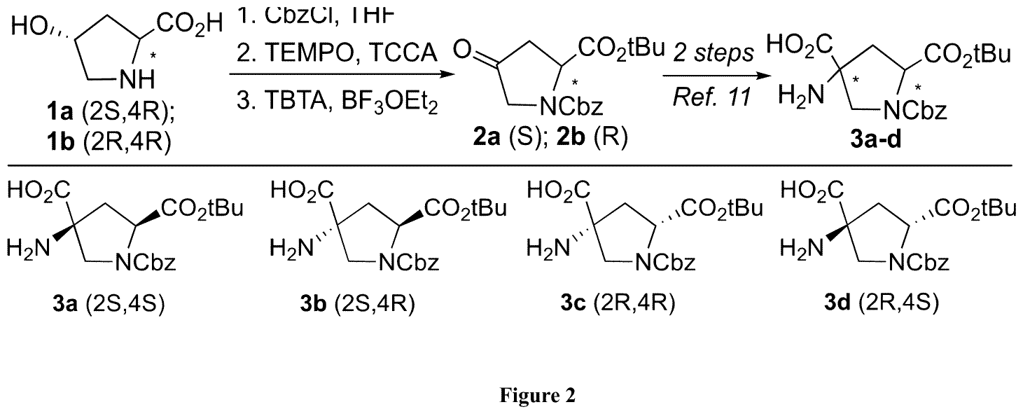

To significantly reduce the labor cost of bis-amino acid synthesis and take advantage of economies of scale, a large-scale synthesis towards the key ketone intermediates 2a and 2b was developed. Compared with the previous synthesis (Cheong, J. E., Tetrahedron Letters 2016, 57 (44), 4882-4884), the presently utilized route increased the intermediate synthesis scale by three orders of magnitude (FIG. 2). The scaled-up synthesis eliminated the use of highly toxic Jones reagent (Caron, S. et al, Chemical Reviews 2006, 106 (7), 2943-2989), replacing it with a (2,2,6,6-tetramethylpiperidin-1-yl)oxyl (TEMPO)-mediated trichloroisocyanuric acid (TCCA) oxidation that avoided downstream problems with impurities that was encountered at a small scale.

The scaled-up synthesis also eliminated explosive isobutylene gas (Wright, S. W. et al., Tetrahedron Letters 1997, 38 (42), 7345-7348), which was a problem at a pilot plant scale, and utilized tert-butylating agent, tert-butyl 2,2,2-trichloroacetimidate (TBTA) instead. In less than two months, tens of kilograms of stereochemically pure S- and R-enantiomers with a cost under $4 per gram were produced in a greener way than previous syntheses.

Following the protocol developed earlier (Cheong, J. E., Tetrahedron Letters 2016, 57 (44), 4882-4884), the Bucherer-Bergs reaction converted 2a and 2b each into a mixture of diastereomeric hydantoins with a roughly 5:1 ratio of (2S,4S) and (2S,4R) stereoisomers respectively for 2a. A large-scale flash chromatography system was utilized using a methylene chloride/isopropanol gradient to separate ˜200 g crude hydantoin diastereomers in one loading. A solvent reclamation system was used to recycle the methylene chloride. Each isolated, stereochemically pure hydantoin product was then hydrolyzed to afford the four optically pure bis-amino acids (3a-d) as previously reported (Cheong, J. E., Tetrahedron Letters 2016, 57 (44), 4882-4884). The detailed multi-kilogram scale synthesis is described below.

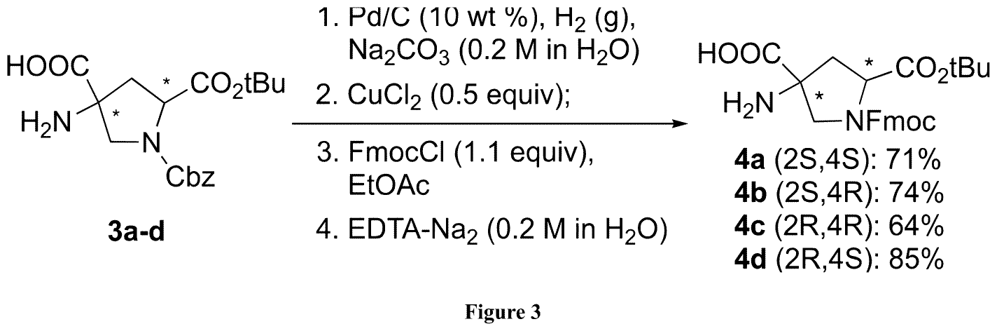

Because the Fmoc group is sensitive to base, the Fmoc protection of the building blocks needs to take place after hydrolysis of the hydantoin and the removal of the Cbz group. Inspired by the selective @-amino protection of lysine (Malkar, N. B et al., Letters in Peptide Science 2000, 7 (5), 263-267), efficient synthesis to install Fmoc group on proline nitrogen has been developed. It relied on the formation of a dimeric Cu (II) complex containing two carboxylic acids and two α-amino groups which temporarily blocks the primary amino group. The carboxylic acid in the C2 position was protected by a tert-butyl group which prevents it from forming a complex with copper together with the amine in the C1 position. Thus, the free primary amine and carboxylic acid in the C4 position was blocked by complexing with Cu²⁺. The proline nitrogen in the building block was then protected with Fmoc protecting reagents, such as Fmoc-Cl or Fmoc-OSu. The copper complex was then dissociated by a strong chelating agent in the final stage of the exchange. This strategy was applied to the four bis-amino acid stereoisomers (3a-d; FIG. 3).

The Cbz deprotection was performed by hydrogenolysis with Pd/C in Na₂CO₃ aqueous solution. The product was used without purification in the following Cu (II) complexation step. Half an equivalent of CuCl₂ was added directly to the suspension to form a complex with the free amine and the carboxylic acid at the C4 position of the bis-amino acid, providing a dark blue solution mixed with Pd/C powder. Under the Schotten-Baumann reaction conditions, a slight excess of Fmoc-Cl in EtOAc was added dropwise to the aqueous slurry. This biphasic mixture was vigorously stirred for 2 hours before filtering out the Pd/C using a short Celite plug. The top EtOAc layer turned blue with much higher intensity than the lower aqueous phase, indicating the formation of Fmoc-protected Cu-complex, which resided in the organic phase due to its reduced polarity. The impurities in the aqueous phase were removed by a separation funnel. The collected organic phase was stirred with ethylenediaminetetraacetic acid (EDTA) aqueous solution to remove Cu²⁺.

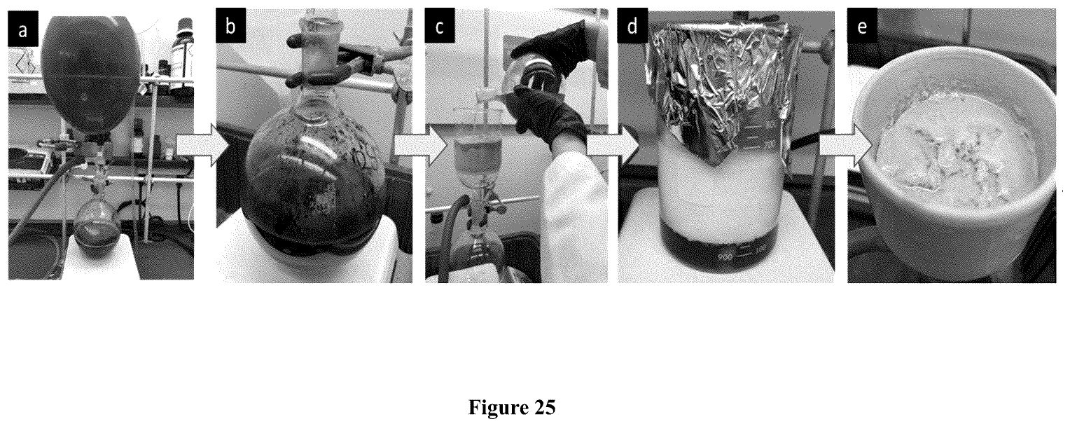

After 12 hours, the blue color was fully transferred from the organic phase to the aqueous phase, suggesting the migration of Cu²⁺ to the latter. Once the Fmoc protected bis-amino acid building blocks 4a-d were released from the copper complex, they precipitated from the biphasic system to form a white crystalline powder, as shown in FIG. 25. The pure mono-protected solid products 4a-d were then separated by vacuum filtration, while a small amount of unreactive starting materials and impurities were left in the yellowish EtOAc phase, and Cu-EDTA byproduct in the blue aqueous phase. This four-step procedure involved no chromatography.

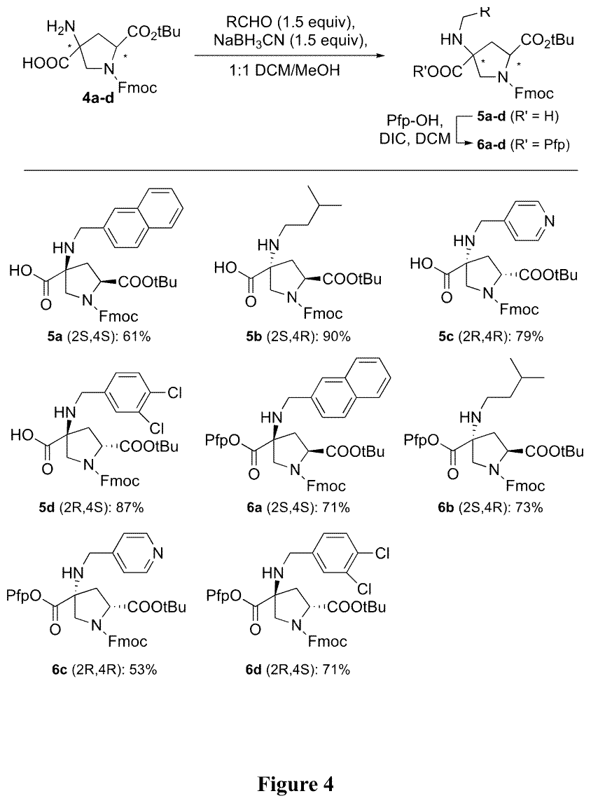

Prior to solid phase assembly, reductive alkylation was used to incorporate various functional groups into the building blocks, by treating them with the corresponding aldehydes and mild reducing agent NaBH₃CN. This was an advantageous feature of spiroligomer synthesis when a functional group chosen from a large set of aldehydes can be installed in the stereochemically pure building blocks to form a measured amount of each monomer for solid phase synthesis of spiroligomers. Each Fmoc bis-amino acid diastereomer was alkylated with a different functional group to obtain the functionalized building blocks 5a-d (FIG. 4).

The four side chains were selected to represent a broad range of functionalities including alkyl, fused aromatic, heterocyclic, and aryl halide groups. For the functionalization and Pfp ester activation of the Fmoc building block, it was found that the previous methods used for pNZ, Boc and Cbz building blocks were compatible with the new Fmoc building blocks (Pfeiffer, C. T. et al, Tetrahedron Letters 2018, 59 (30), 2884-2888; Brown, Z. Z et al., Biopolymers 2011, 96 (5), 578-585).

By adding DCM with methanol in a 1:1 ratio, (2S,4R)- and (2R,4S)-diastereomers were dissolved to improve the efficiency of reductive alkylation. Solid loading the crude slurry onto Celite with the help of a large amount of methanol and normal phase chromatography at 0-20% methanol in DCM gradient was found to be efficient to purify the functionalized bis-amino acids, with a yield ranging from 61% to 90%.

Consistent with previous findings, the functionalized secondary amine on the quaternary center of each building block was so sterically hindered that it did not couple at any appreciable rate to activated esters (Brown, Z. Z. et al., Journal of the American Chemical Society 2008, 130 (44), 14382-14383). Therefore, it can be used as a monomer for solid phase synthesis without protecting the secondary amine on 5a-d.

Previously dimerization of the building blocks using traditional in-situ activation coupling strategies was observed (Brown, Z. Z. et al., Journal of the American Chemical Society 2008, 130 (44), 14382-14383). Dimerization was minimized using Pentafluorophenol (Pfp-OH) to pre-activate the monomers and obtain the bench-stable building blocks 6a-d as shown in FIG. 4 (Pfeiffer, C. T. et al, Tetrahedron Letters 2018, 59 (30), 2884-2888). In the coupling step, the Pfp esters of the building blocks can be added directly to the amine on solid support to avoid the formation of symmetric dimers. Each monomer was purified by normal phase column chromatography at 0-50% Hexane/EtOAc and stored at −20° C. until needed.

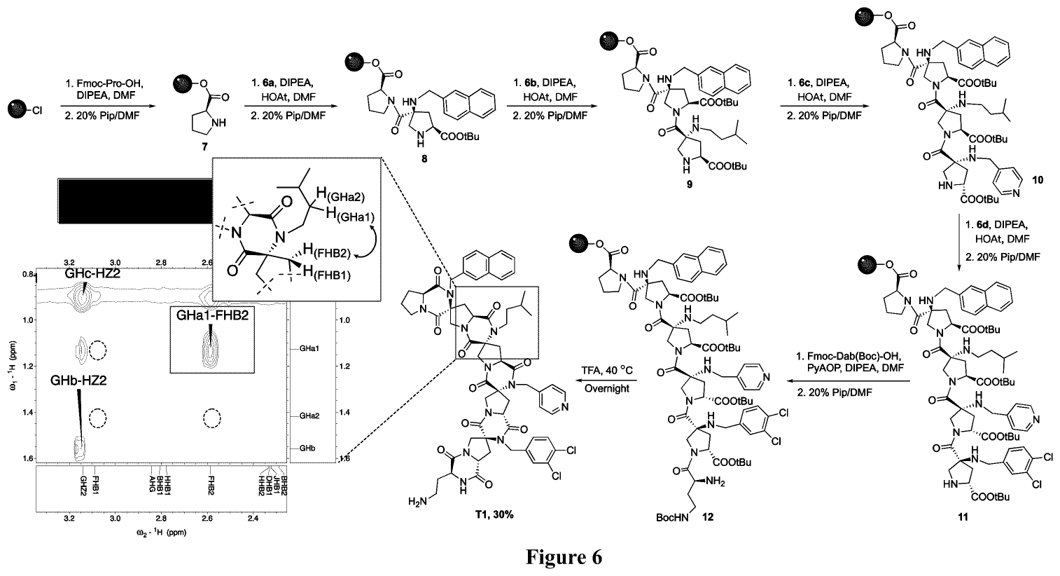

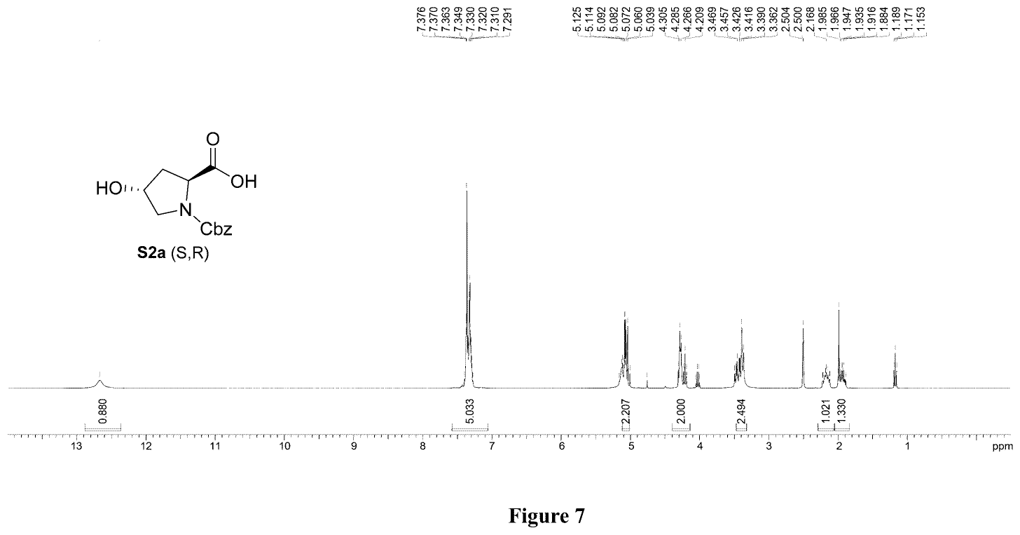

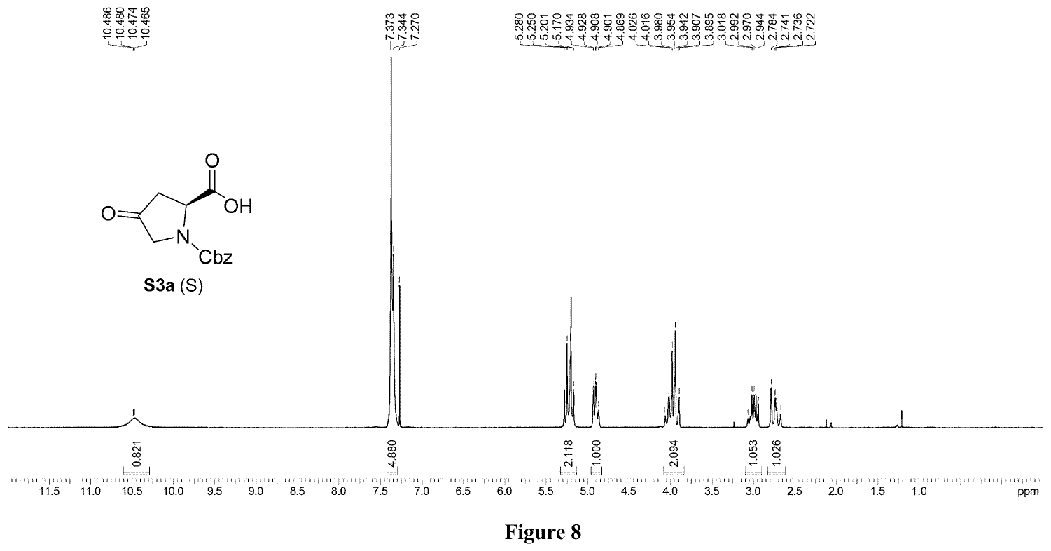

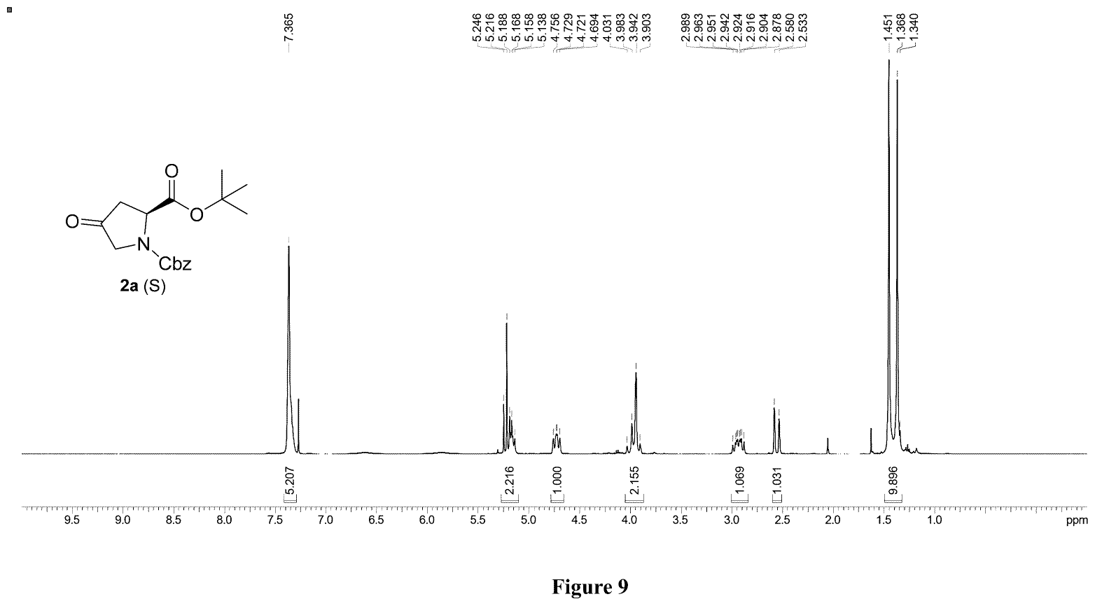

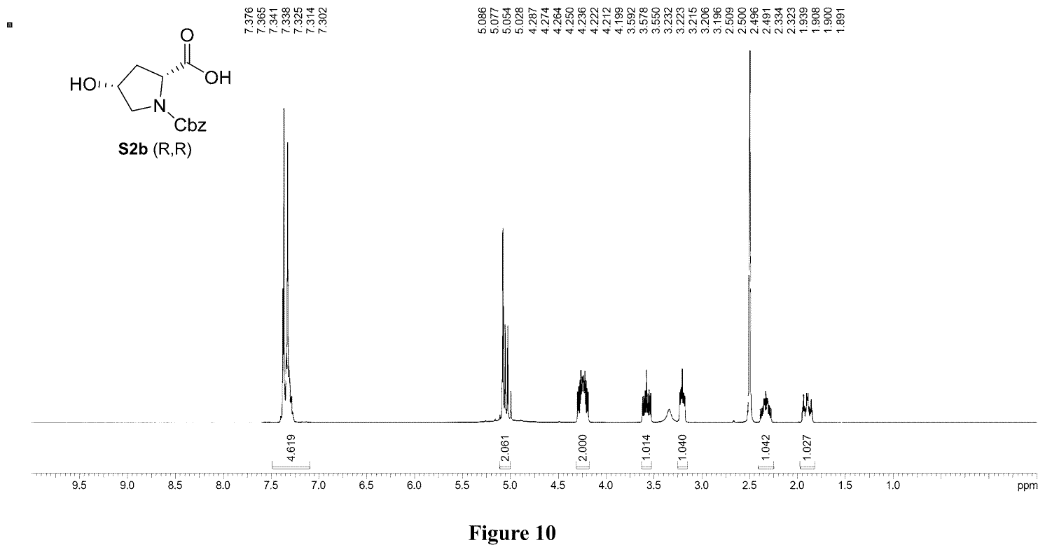

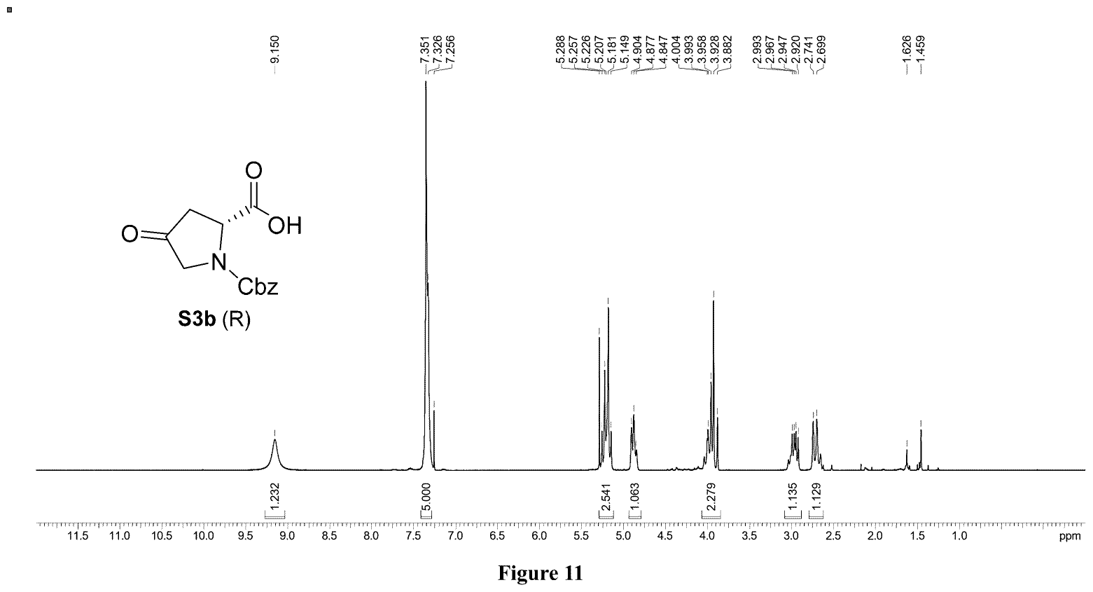

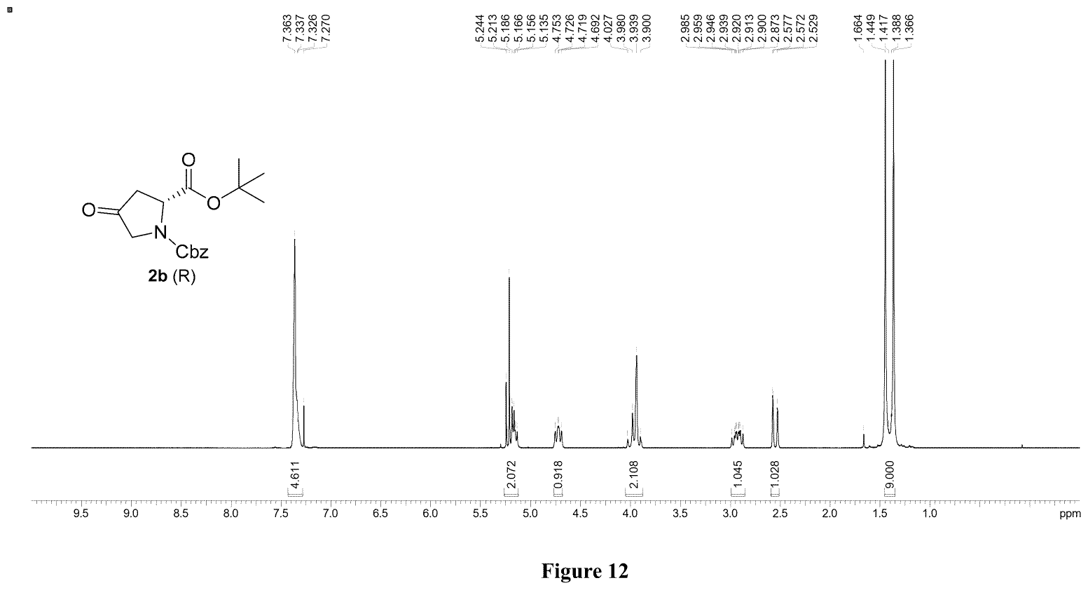

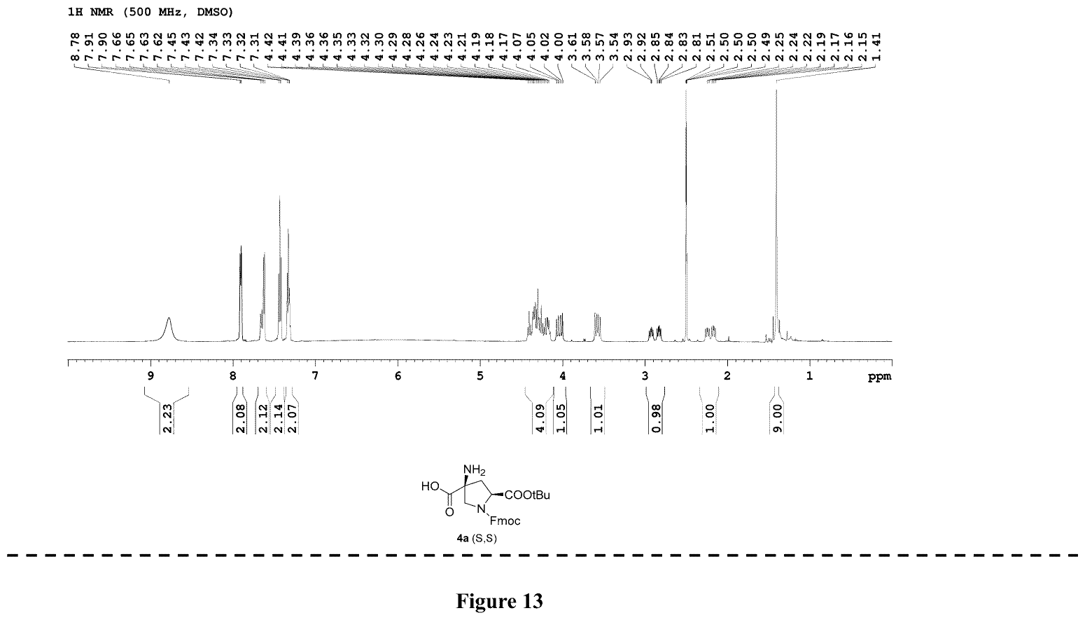

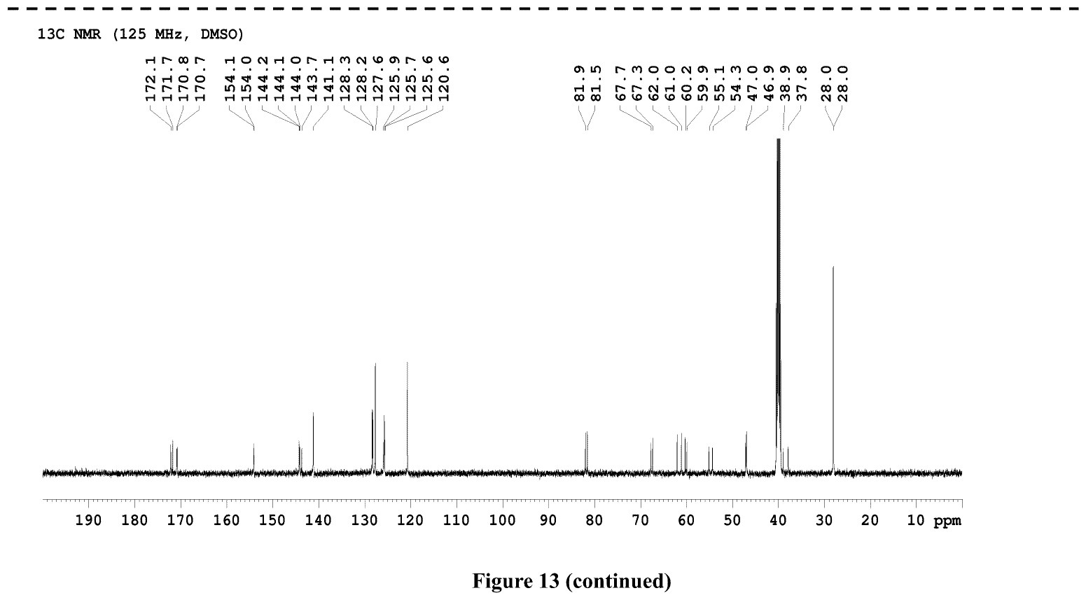

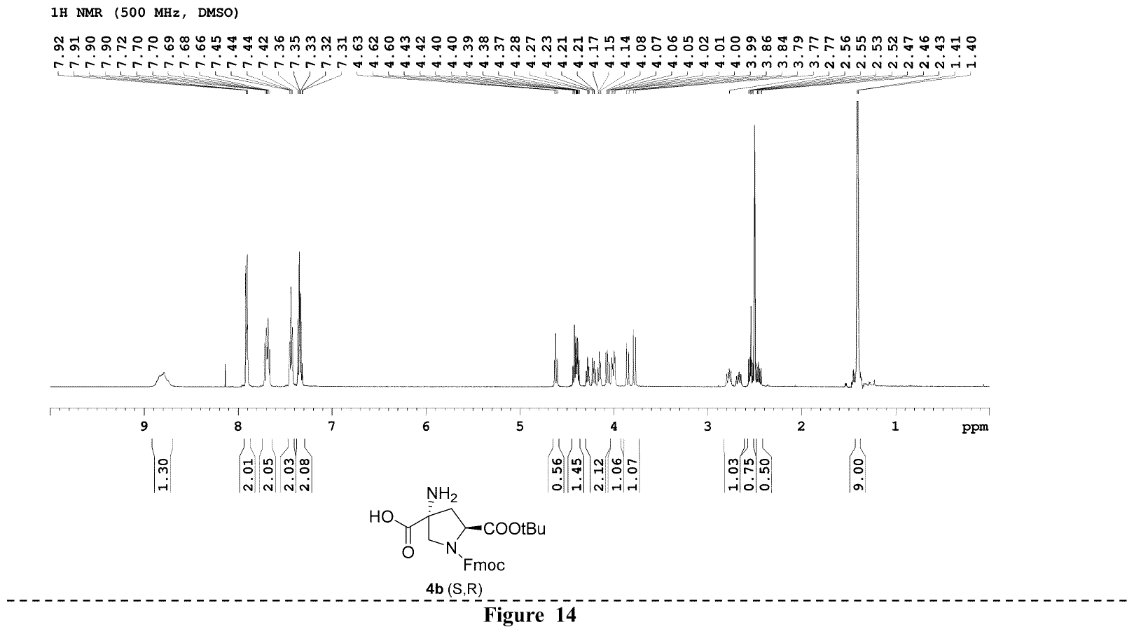

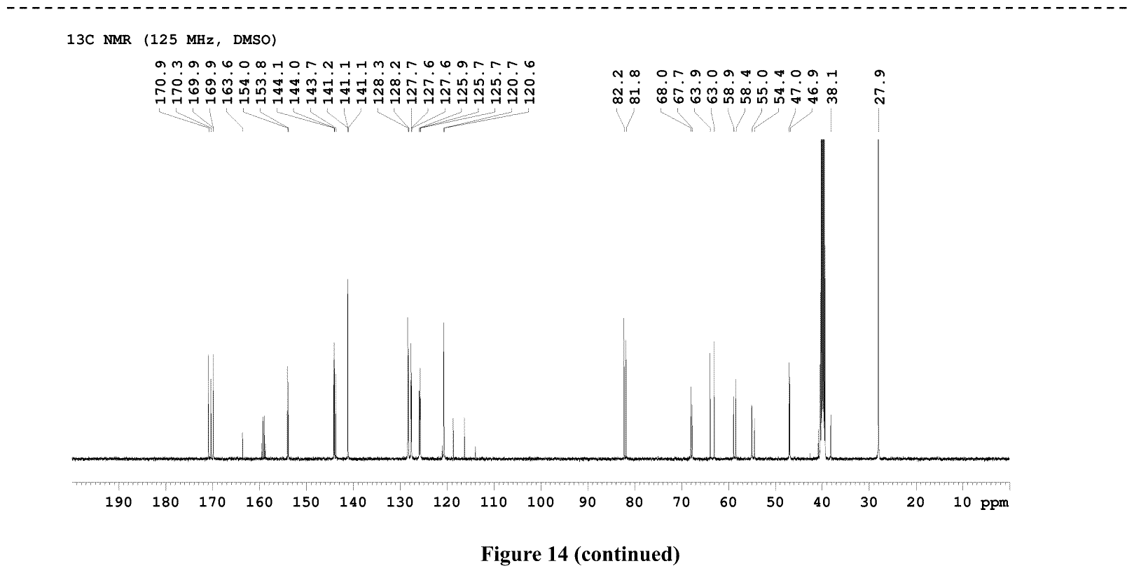

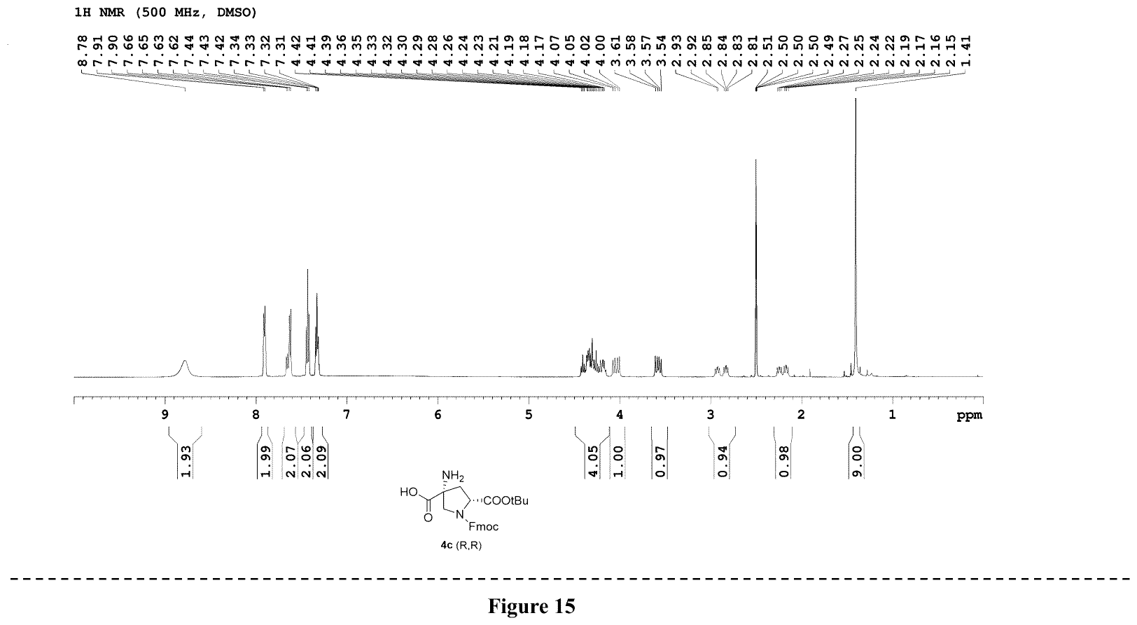

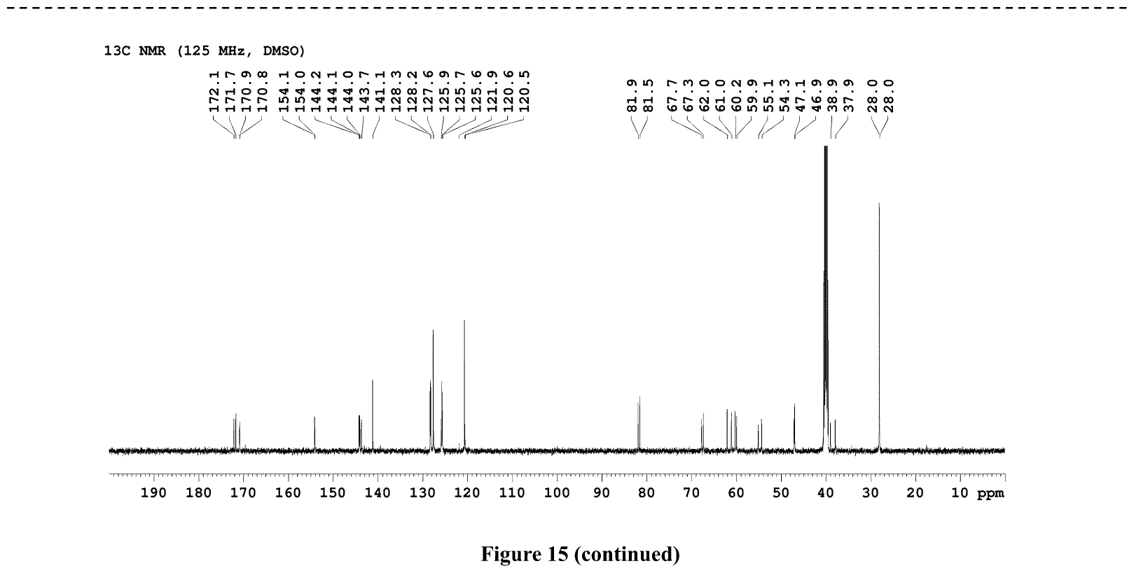

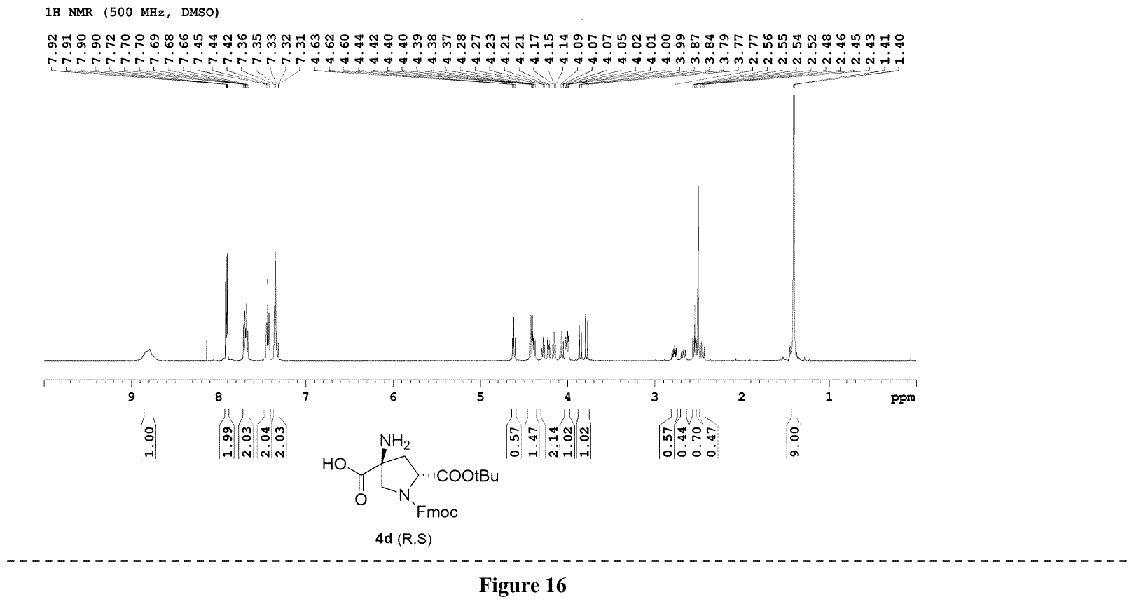

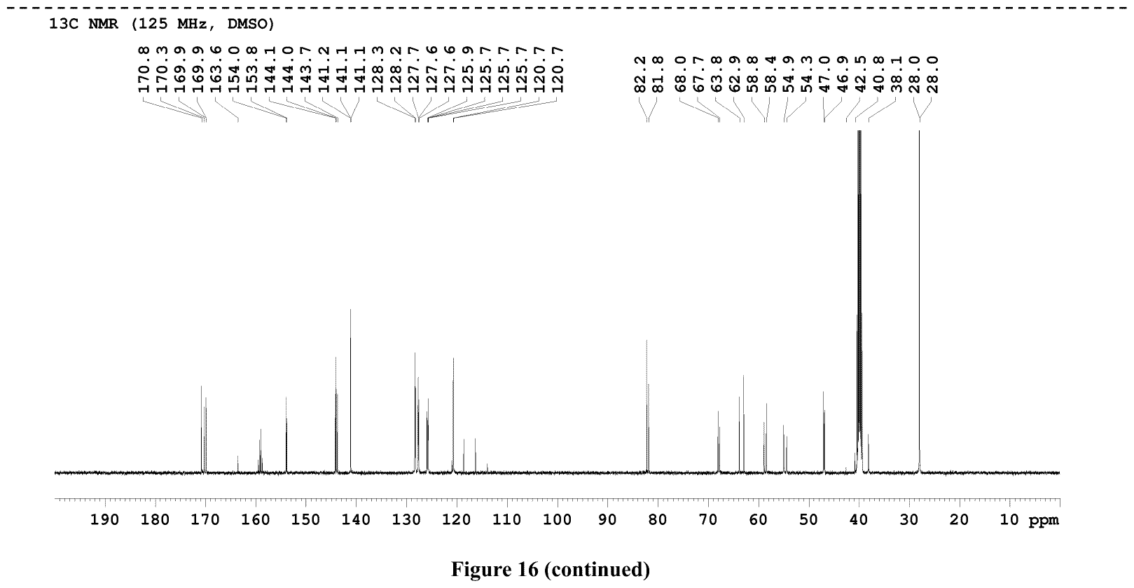

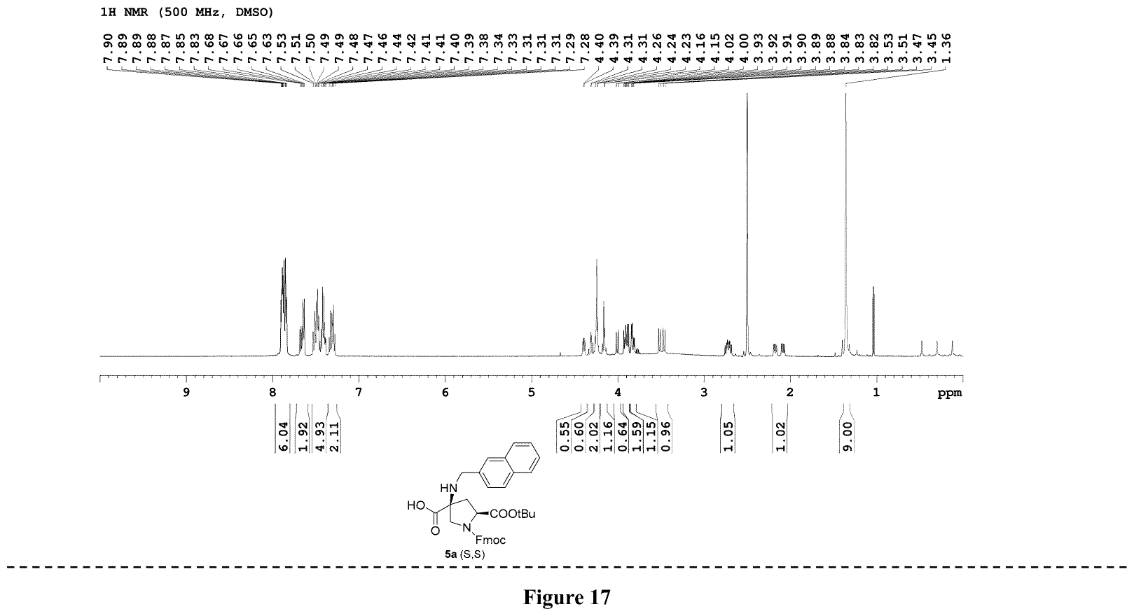

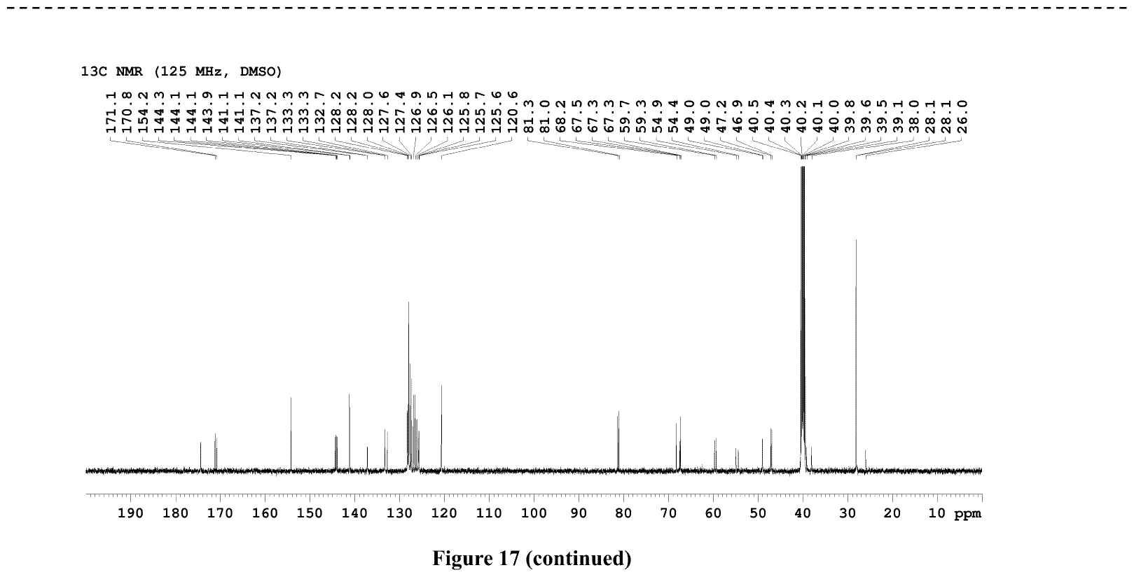

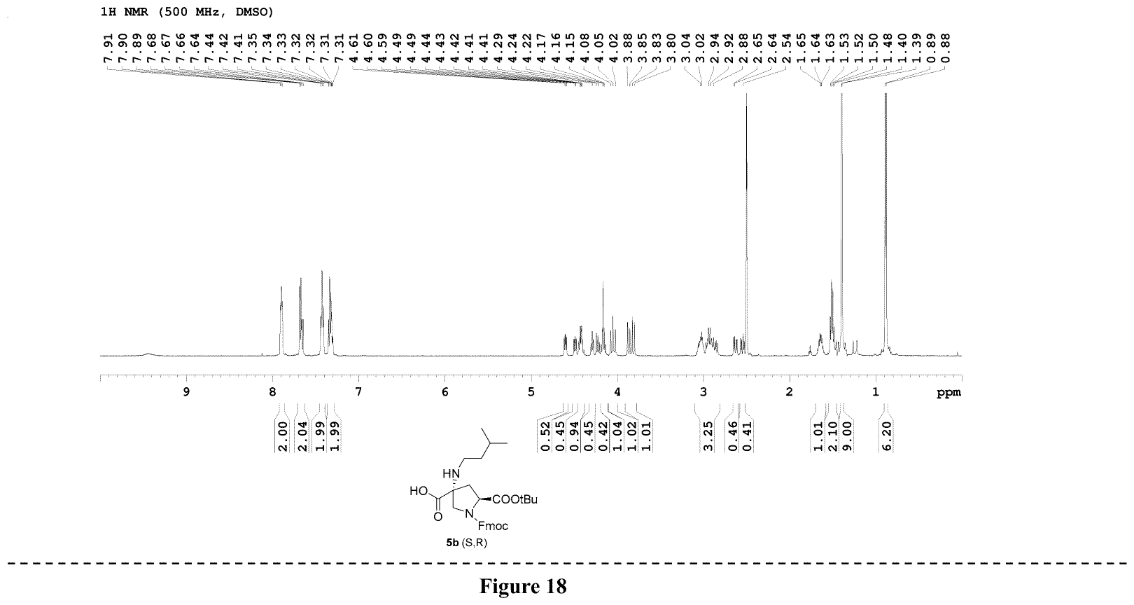

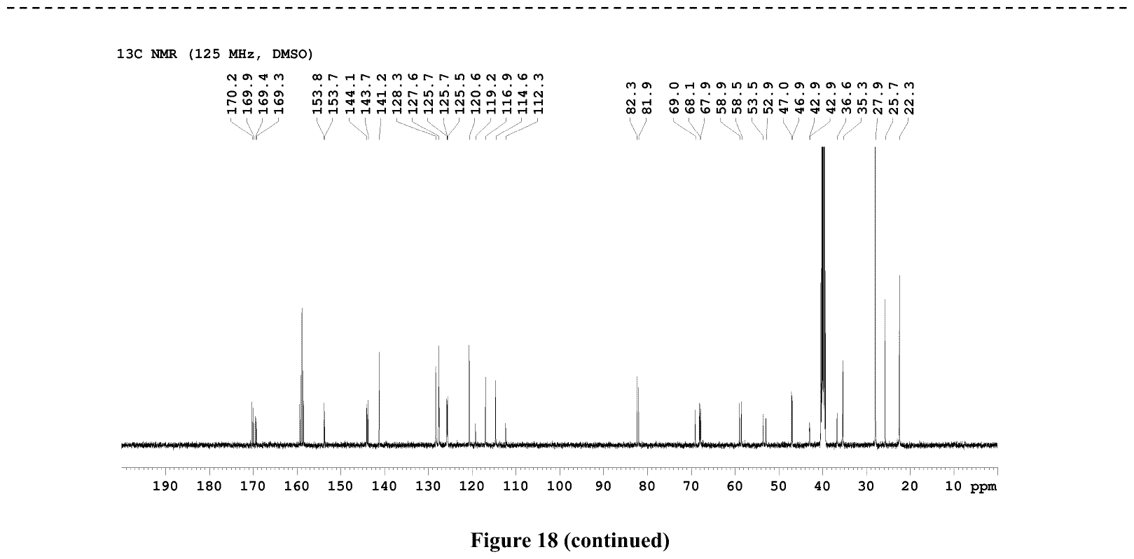

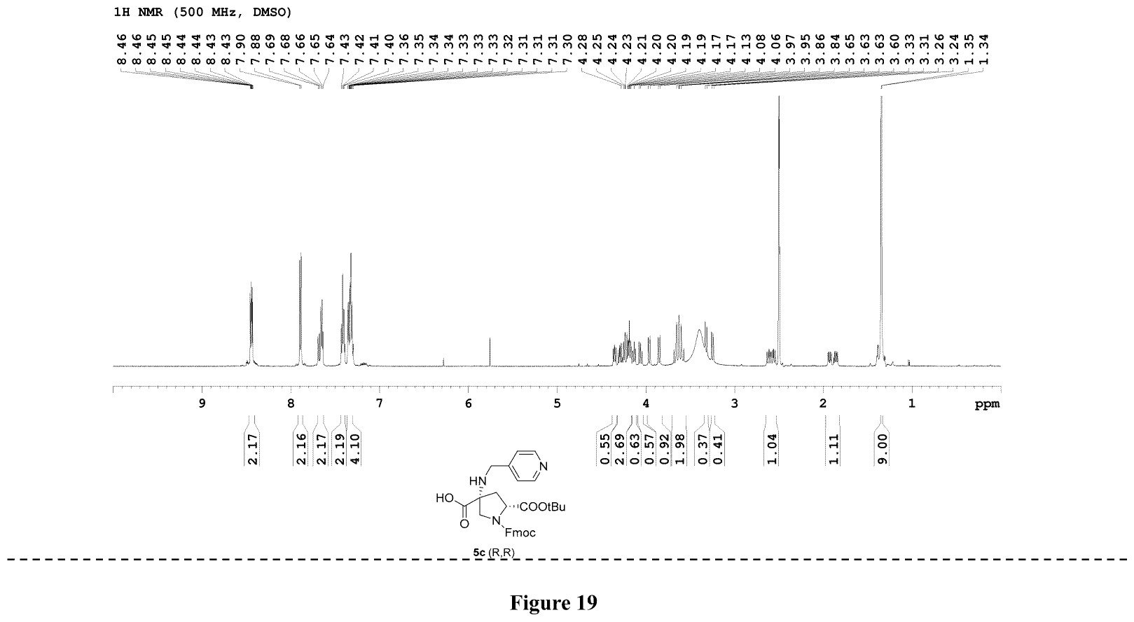

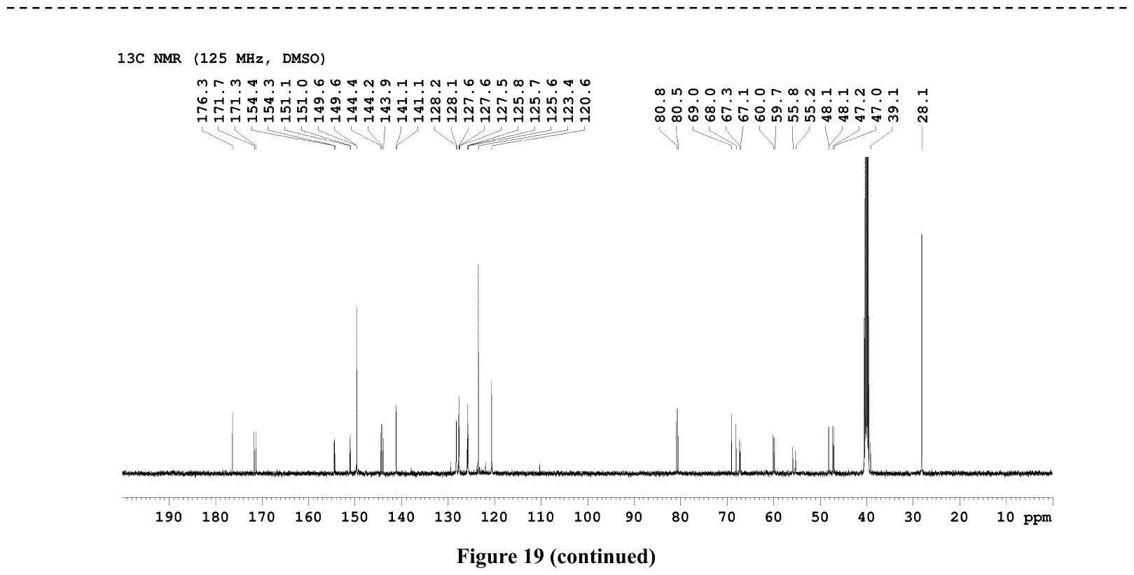

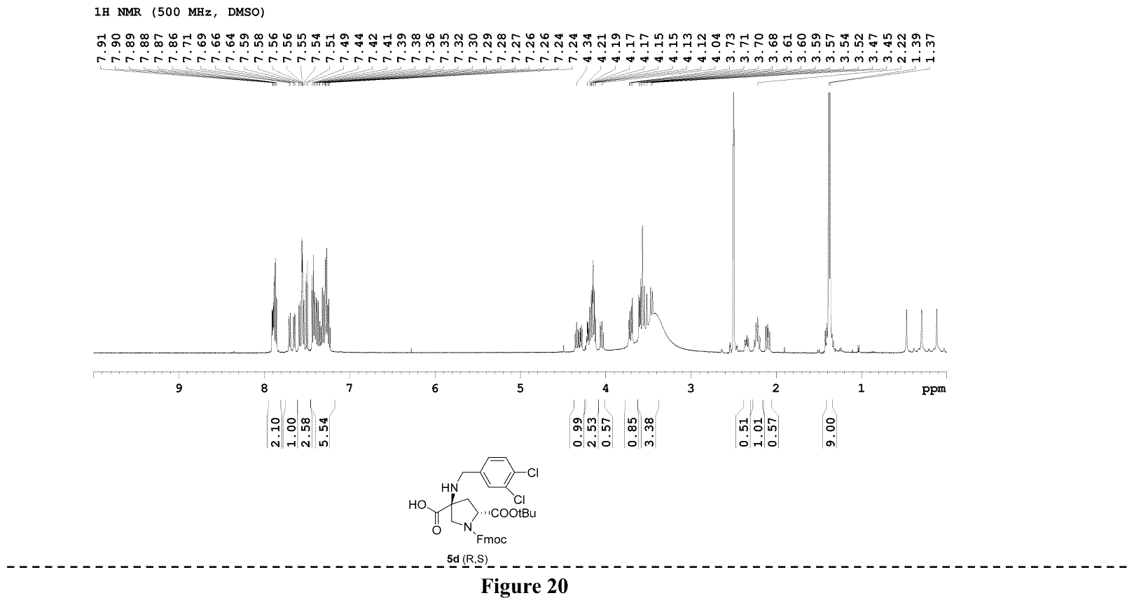

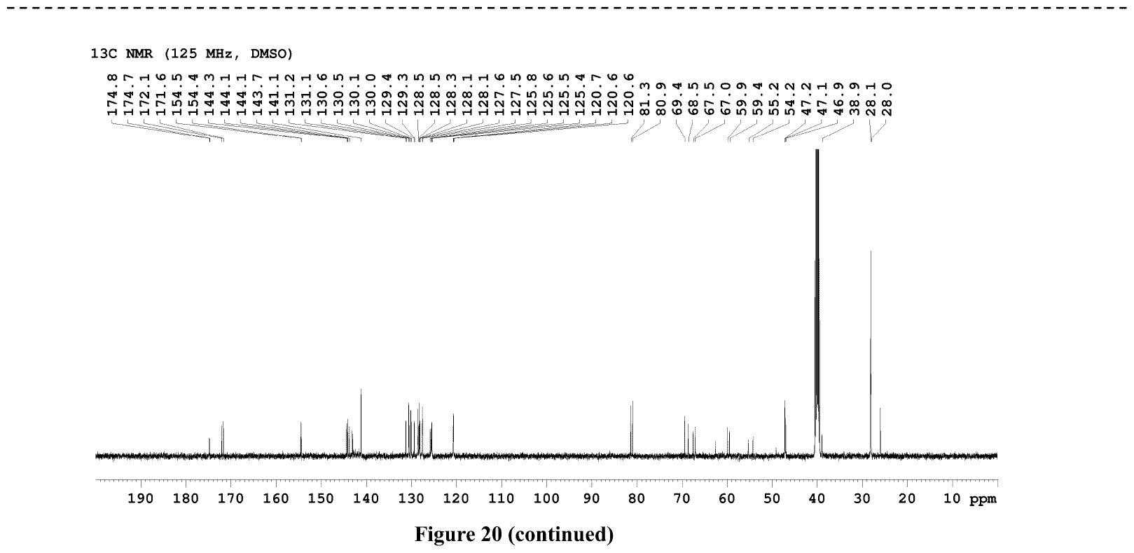

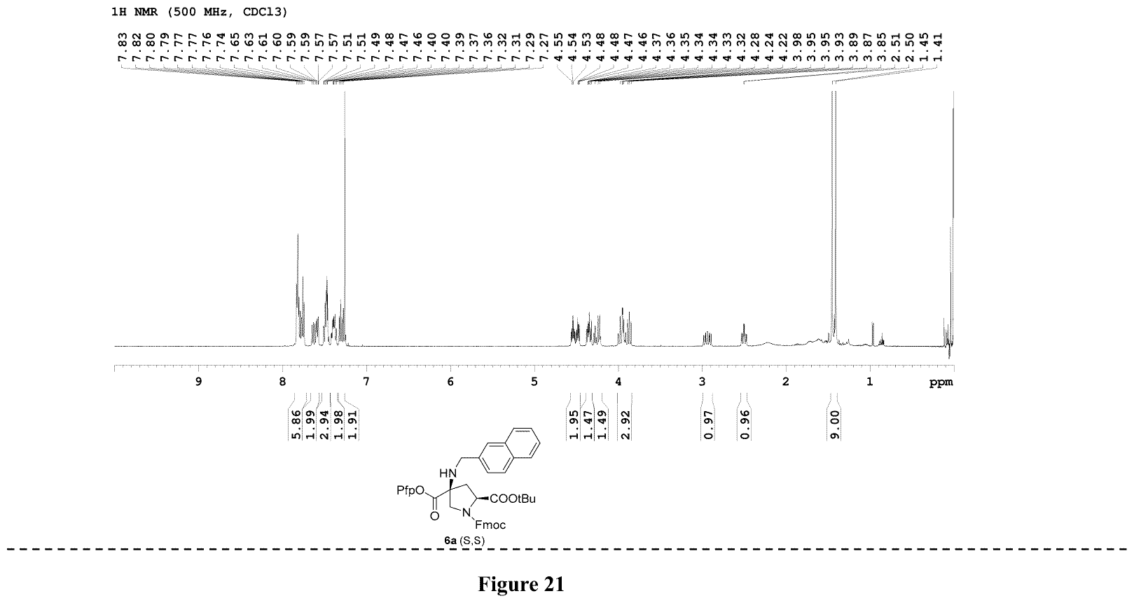

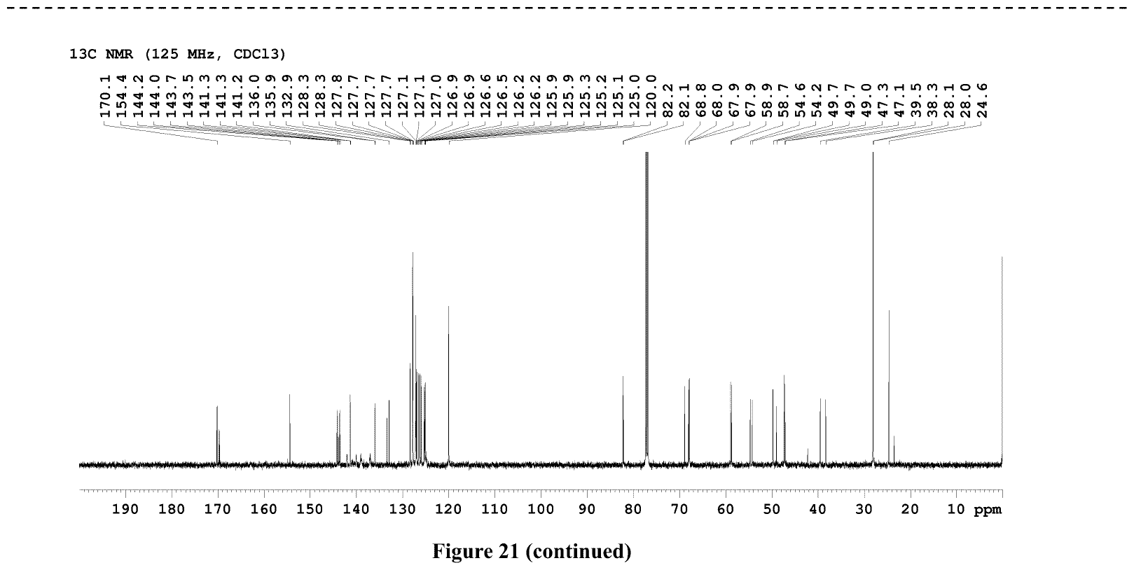

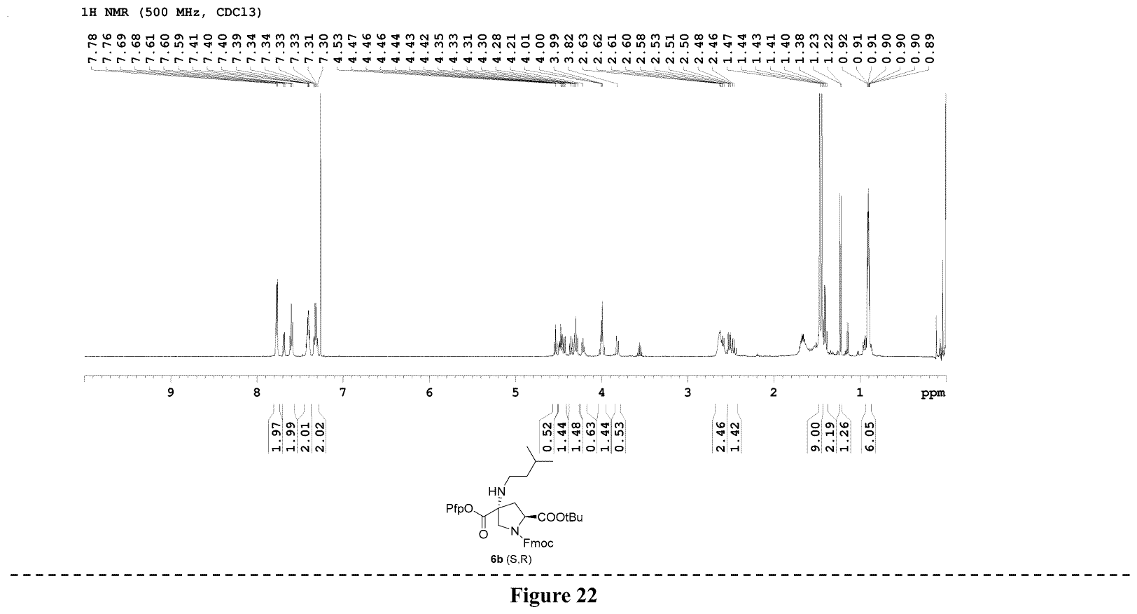

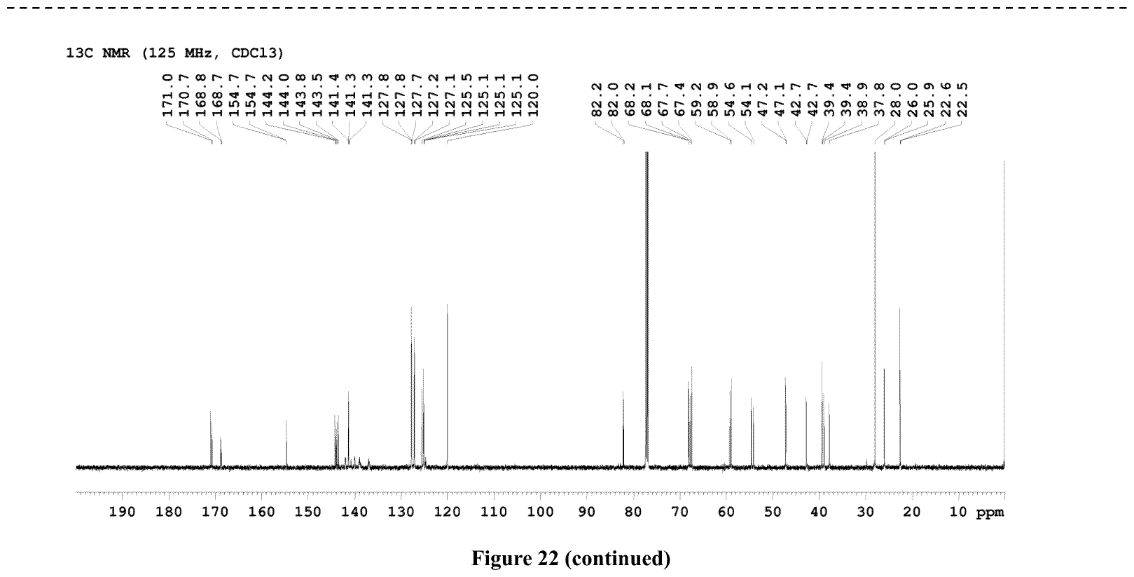

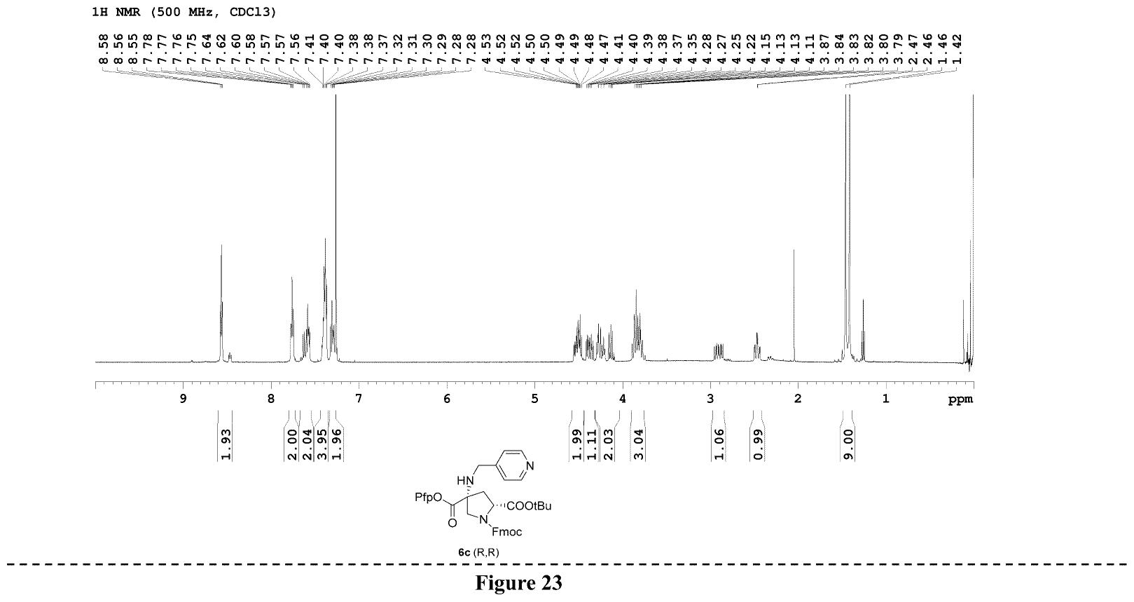

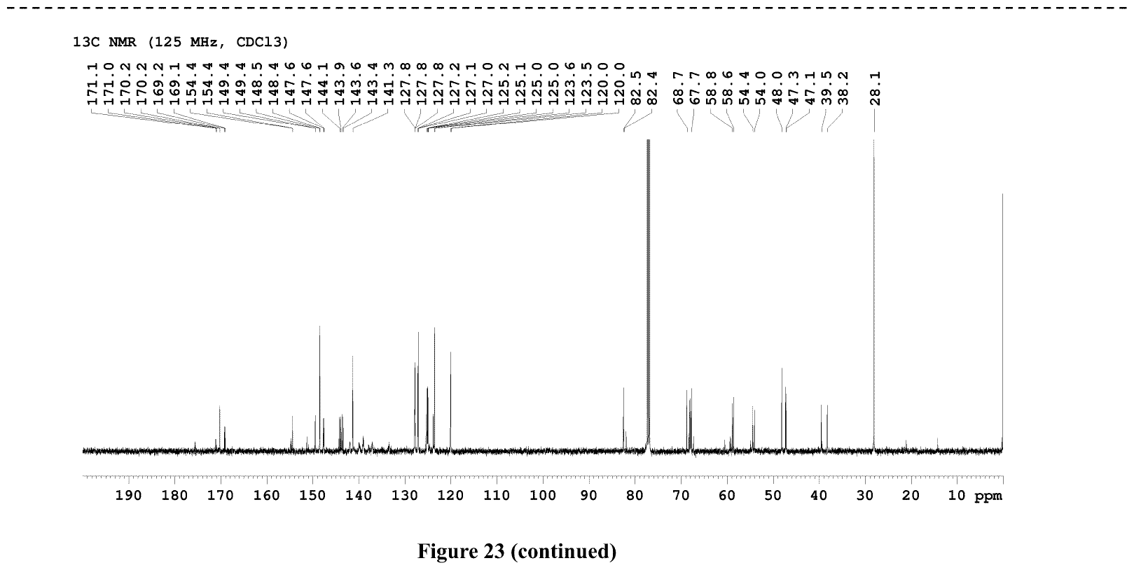

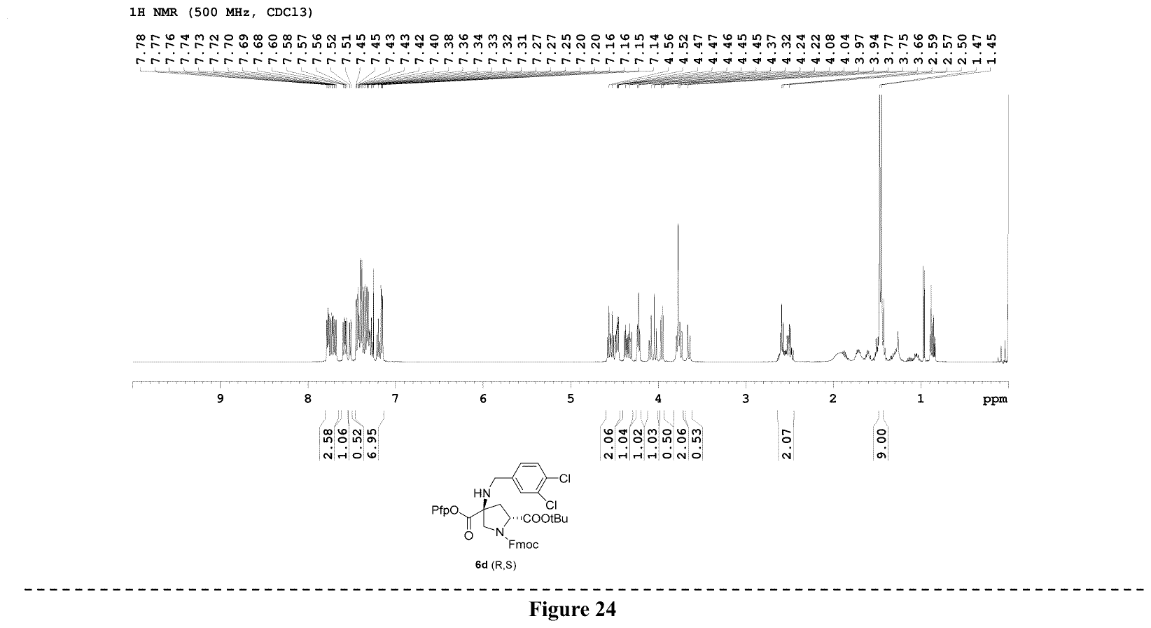

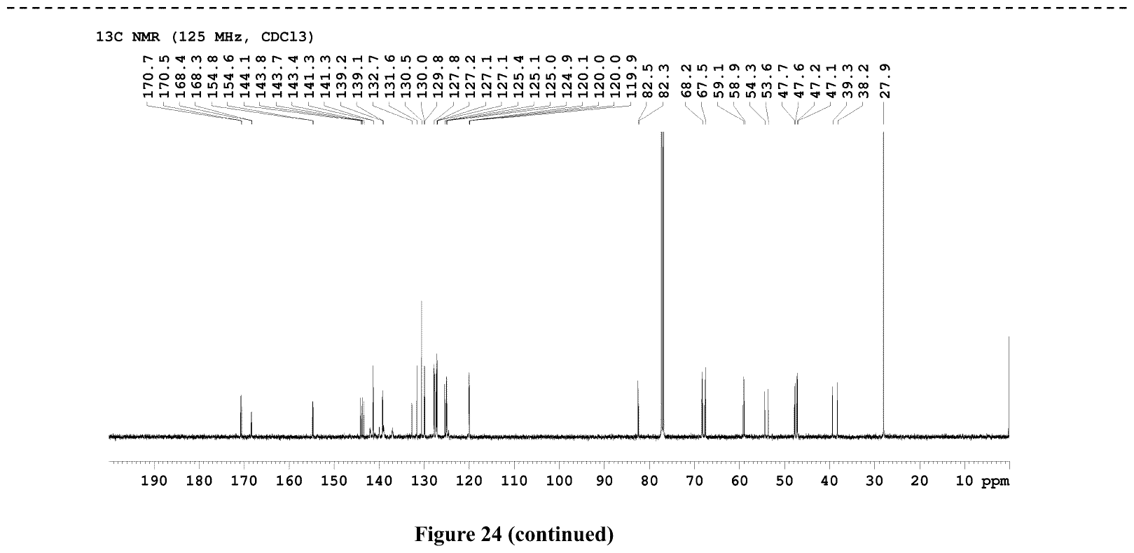

Highly functionalized spiroligomers were assembled through solid phase synthesis with the four stereoisomers of the building blocks 6a-d on a semi-automated microwave peptide synthesizer. As shown in FIG. 6, L-proline was first loaded onto 2-chlorotrityl chloride resin using N,N-diisopropylethylamine (DIPEA), followed by deprotection of the Fmoc group to generate 7. The second residue, Pfp ester 6a was coupled using a similar HOAt/DIPEA protocol developed for the pNZ building blocks (Pfeiffer, C. T. et al, Tetrahedron Letters 2018, 59 (30), 2884-2888).

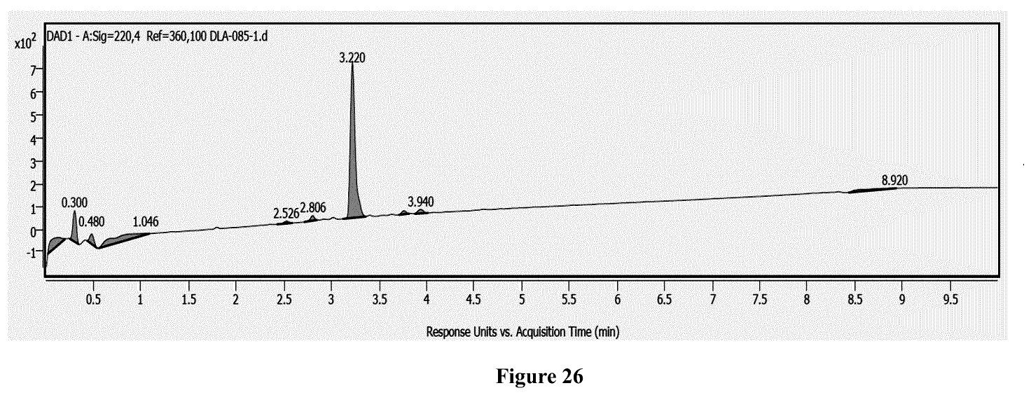

After optimization, it was discovered that 2 equiv. of building block was sufficient to complete the coupling at 50° C. for 1 hour in the presence of 4 equiv. of HOAt and 8 equiv. of DIPEA. Excess base was used to balance the acidity of excess HOAt, which prematurely cleaved the extremely sensitive chloro-trityl linker. Coupling of building block 6b onto the resin was followed by the Fmoc removal to obtain 9. The coupling of the next bis-amino acid 6c was followed by the removal of the Fmoc group to obtain 10. This process was repeated for the installation of the last building block 6d, forming compound 11. The sequence was capped by Fmoc-Dab (Boc)-OH to provide 12, which was liberated from the solid phase by exposure to TFA. After heating the TFA solution containing the cleaved product 12 at 40° C. overnight, the DKPs in tetramer T1 were fully formed presumably through an acid-catalyzed condensation. No significant byproduct was observed in the crude HPLC (FIG. 26). The final yield of T1 was 30% after preparative HPLC based on the maximum loading of resin.

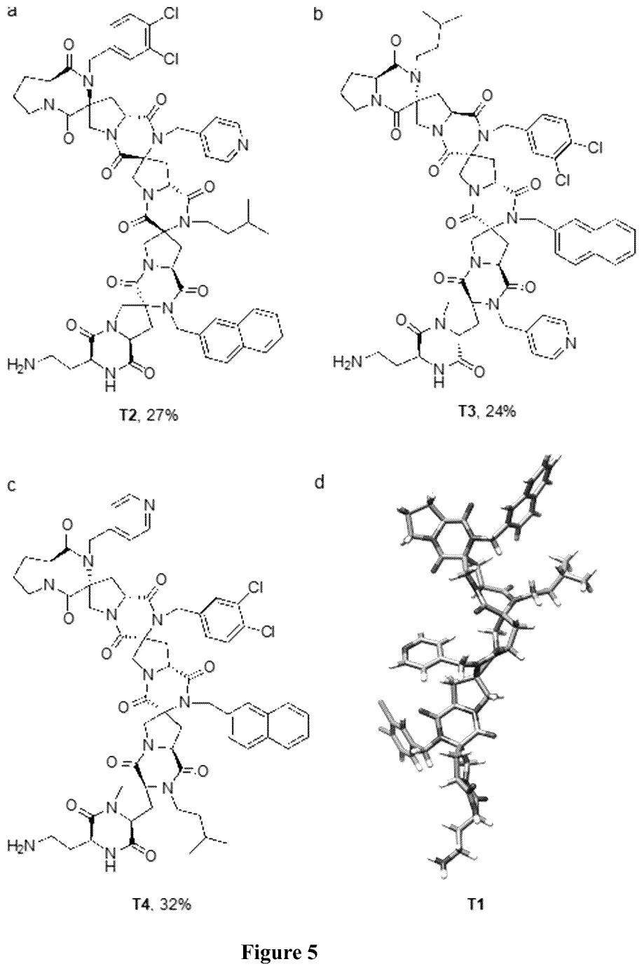

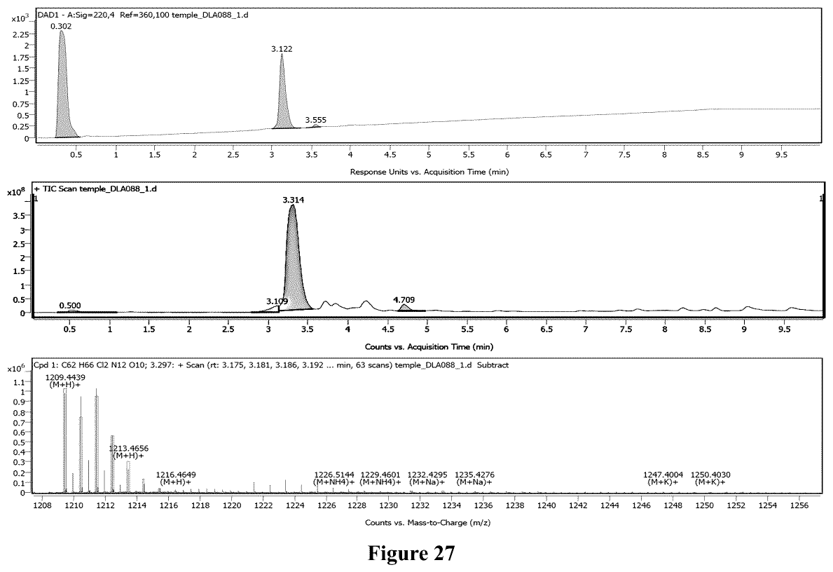

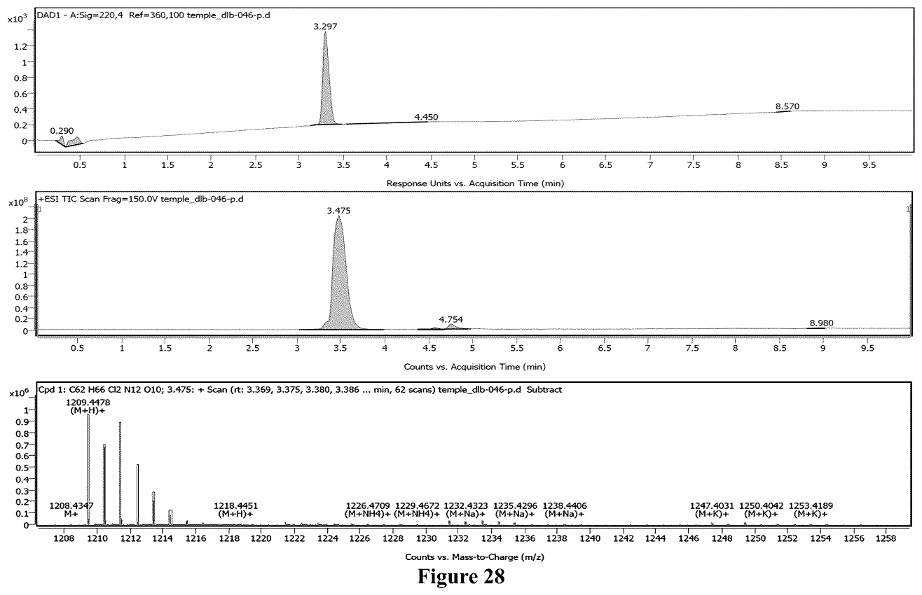

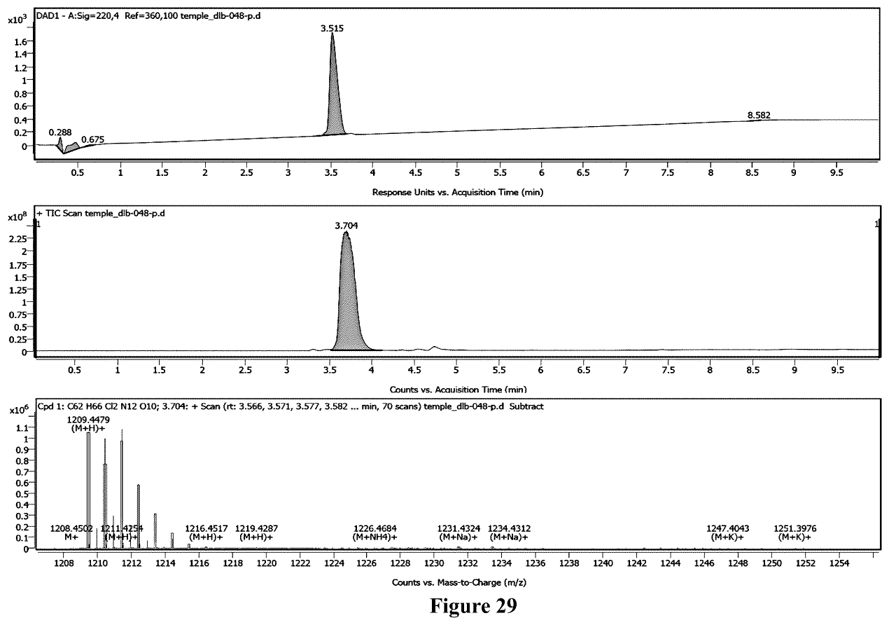

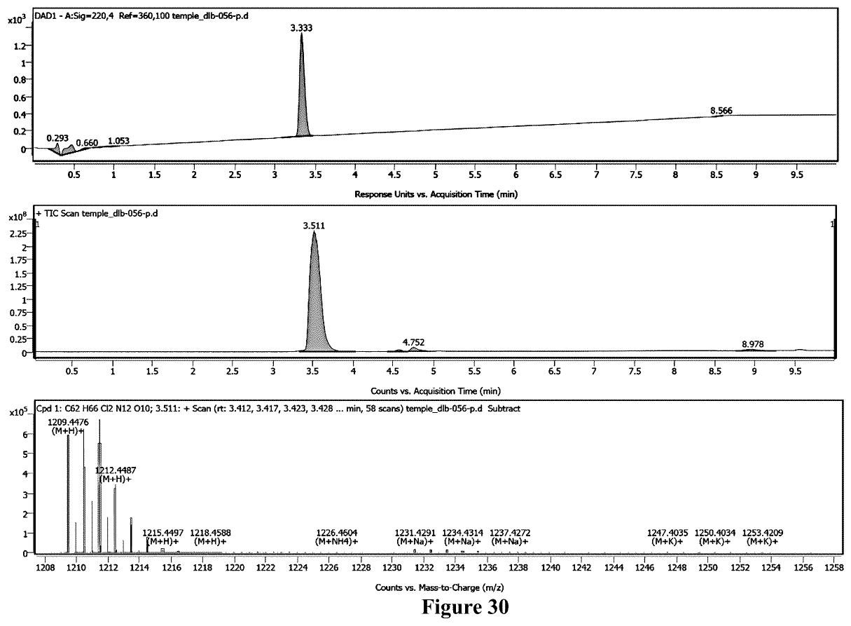

To demonstrate the generality of this synthetic approach, three other tetramers, T2-T4 were synthesized by altering the position of building blocks (FIG. 5). The composition of T1-T4 was verified by high-resolution mass spectrometry (QTOF MS) (FIG. 27 through FIG. 30). Two-dimensional NMR experiments: double quantum filtered-correlated spectroscopy (DQF-COSY), heteronuclear single quantum coherence spectroscopy (HSQC), heteronuclear multiple bond correlation spectroscopy (HMBC) and heteronuclear multiple quantum coherence (HMQC) in DMSO-d6 were carried out and used to assign the ¹H and ¹³C resonances with the software package SPARKY. The expected connectivity was confirmed with the cross peaks from the correlations between neighboring building blocks in a band selective HMBC spectrum. Rotating frame Overhauser effect spectroscopy (ROESY) correlations were used to identify the relative stereochemistry of C2 and C4 hydrogens in each pyrrolidine and they confirmed their configurations relative to the configuration of the C1 carbon on each building block.

Furthermore, all side chains had hydrogens that showed ROESY correlations with backbone hydrogens, and they were consistent with the side chain having a preferred orientation. For example, as shown in FIG. 6, one of the β protons, FHB2, below the plane of the second bis-amino acid proline ring, had a strong ROESY correlation with GHa1 (solid red box), which was one of the methylene protons in the side group of the neighboring building block. On the other hand, no correlation was found between GHa1-FHB1, GHa2-FHB1, or GHa2-FHB2 (expected where blue dashed circles are drawn, FIG. 6). This indicated that the rotation of side chains was restricted by the interaction with the rigid backbones, which met the expectation of well-defined 3D structures. The energy-minimized structure of T1 is also shown in FIG. 5.

In summary, the present studies have demonstrated a scaled up synthesis of bis-amino acid intermediates to tens of kilogram scale. Cu²⁺ complexation strategy was successfully used to selectively incorporate an Fmoc group in a four-step, one-pot process with no chromatography with excellent yield and purity. Using these monomers, four spiroligomers were synthesized on solid support with four different building blocks containing unique functional groups and stereochemistry. This work laid the foundation for the reliable and automated assembly of highly functionalized spiroligomer libraries.

Overall, the present studies described the fluorenylmethoxycarbonyl (Fmoc)-protection of functionalized bis-amino acid building blocks using a temporary Cu2+ complexation strategy together with an efficient multi-kilogram-scale synthesis of bis-amino acid precursors. This allowed the synthesis of stereochemically and functionally diverse spiroligomers utilizing a solid-phase Fmoc/tBu chemistry to facilitate the development of applications. Four tetramers were assembled on a semi-automated microwave peptide synthesizer. The secondary structures with two-dimensional nuclear magnetic resonance spectroscopy were also determined.

The materials and methods employed in the present experimental examples are now described.

### General Procedures

Reactions were performed in standard and oven-dried glassware equipped with PTFE-coated magnetic stir bars. Stainless steel syringes were used to transfer air- and moisture-sensitive liquids. Reported concentrations refer to solution volumes at room temperature. Evaporation and concentration in vacuo were performed using rotary evaporators.

### Materials

Reagents were purchased in reagent grade from commercial suppliers and used without further purification, unless otherwise described. Compounds S2a, S3a, 2a, S2b, S3b, and 2b were synthesized (Levins, C. G. et al., Journal of the American Chemical Society 2003, 125 (16), 4702-4703). The ¹H NMR spectra were very clean and compared well to the published NMR spectra. Compounds 3a-d were synthesized following the previously reported procedure (Cheong, J. E. et al., Tetrahedron Letters 2016, 57 (44), 4882-4884).

### Instrumentation

Low-resolution high-performance liquid chromatography-mass spectrometry (LR-HPLC-MS) analysis was performed on an Agilent 1290 liquid chromatography system with a Supelco Ascentis® Express C8 column (2.7 μm packing, 2.1×50 mm) using a water-acetonitrile gradient solvent system containing 0.1% formic acid at a flow rate of 1.0 mL/min. This system was connected to an Agilent 6120 single quadrupole mass spectrometer that utilizes electrospray ionization.

High-resolution HPLC-MS was performed on an Agilent Infinity II series LCMS system with an Agilent Poroshell 120 EC-C18 column (1.9 μm packing, 2.1×50 mm) using a water-acetonitrile gradient containing 0.1% formic acid at a flow rate of 1.0 mL/min. This system was coupled to a 6000 series quadrupole time-of-flight (QTOF) mass spectrometer.

Preparative HPLC was performed on an Agilent Infinity II series LC/MSD system with a Phenomenex Aeris Peptide XB-C18 column (5 μm packing, 21.2×150 mm) using a water-acetonitrile gradient solvent system containing 0.1% trifluoroacetic acid at a flow rate of 25 mL/min with a single quadrupole mass spectrometer that utilizes electrospray ionization.

¹H and ¹³C one-dimensional nuclear magnetic resonance (NMR) experiments were performed on a Bruker Avance 500 MHz instrument at 25° C. Chemical shifts are reported relative to residual solvent peaks or tetramethylsilane. Data are represented as follows: chemical shift, integration, multiplicity (br=broad, s=singlet, d=doublet, t=triplet, q=quartet, qn=quintet, sp=septet, m=multiplet), coupling constants in Hertz (Hz). Some compounds exist at room temperature as a mixture of two slowly interconverting rotamers (on the NMR time scale) due to the tertiary amide. Rotameric peaks are indicated in the NMR data.

Two-dimensional NMR experiments were performed on a Bruker Avance NEO 600 MHz spectrometer equipped with a cryoprobe at the Spectroscopy Support Facility (SSF) at Fox Chase Cancer Center.

Normal-phase purifications were performed on an ISCO® (Teledyne, Inc.) automated flash chromatography system using various sizes of pre-packed RediSep® Rf silica gel columns (60 Å porosity, 230×400 mesh particle size) purchased from Sorbent Technologies.

Hydantoin diastereomers were separated on an Biotage® Isolera LS automated flash purification system using Biotage® SNAP KP-SIL 1500 g columns (55 Å porosity, 50 μm particle size).

Reverse-phase purifications were performed on an ISCO (Teledyne, Inc.) automated flash chromatography system using various sizes of pre-packed RediSep® Rf C18 reversed phase columns (60 Å porosity, 230×400 mesh particle size) purchased from Sorbent Technologies with a water-acetonitrile gradient solvent system containing 0.1% formic acid.

Solid-phase synthesis of spiroligomers was performed on a DiscoverBio semi-automated microwave-assisted peptide synthesizer from CEM Corporation, which is equipped with a 25 mL reaction vessel.

### Software

One-dimensional NMR spectra were processed with Bruker Topspin. Two-dimensional NMR spectra were analyzed with SPARKY (Goddard, T. D.; Kneller, D. G. Sparky 3.190; University of California: San Francisco, CA, 2015). (Lee, W. et al., Bioinformatics 2014, 31 (8), 1325-1327).

Molecular graphics were generated with UCSF Chimera 1.14 (build42094; Pettersen, E. F. et al., Journal of Computational Chemistry 2004, 25 (13), 1605-1612).

The in-house software package CANDO (github.com/cando-developers/cando.git) was used to carry out the energy minimization of the three-dimensional structure of T1 using the Generalized Amber Force Field (GAFF) energy function. (Wang, J. et al., Journal of Computational Chemistry 2004, 25 (9), 1157-1174)

### Abbreviations

DIPEA=diisopropylethylamine, DIC=N,N′-Diisopropylcarbodiimide, ESI=electrospray ionization, HOAt=1-hydroxy-7-azabenzotriazole, TFA=trifluoroacetic acid, THF=tetrahydrofuran, DMF=dimethylformamide, DMSO=dimethyl sulfoxide, MTBE=methyl tert-butyl ether, PyAOP=((7-azabenzotriazol-1-yloxy)tripyrrolidino-phosphonium hexafluorophosphate), Fmoc=fluorenylmethoxycarbony, Boc=tert-butyloxycarbonyl, Cbz-Cl=benzyl chloroformate, Pfp=pentafluorophenyl, EDTA=ethylenediaminetetraacetic acid, Dab=2,4-diaminobutyric acid, pro=proline, NMR=nuclear magnetic resonance, TEMPO=(2,2,6,6-Tetramethylpiperidin-1-yl)oxyl oroxidanyl, TLC=thin layer chromatography, DKP=diketopiperazine, HPLC=high performance liquid chromatography, HR=high-resolution, LC=liquid chromatography, LR=low-resolution, MS=mass spectrometry.

### Experimental Procedures and Characterization Data

### Synthesis and Characterization of Building Blocks

### Synthesis of 2a at Multi-Kilogram Scale

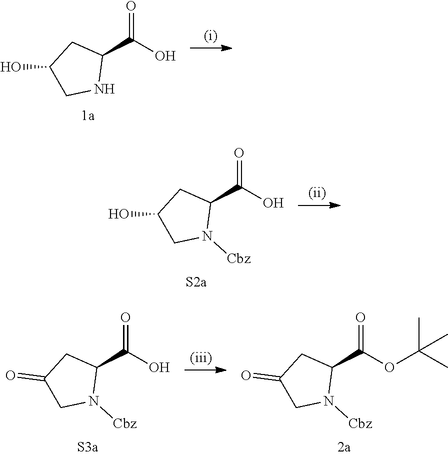 [SMILES: O=C(O)[C@@H]1C[C@@H](O)CN1 ; O=C(O)[C@@H]1C[C@@H](O)CN1C(=O)OCc1ccccc1 ; O=C1C[C@@H](C(=O)O)N(C(=O)OCc2ccccc2)C1 ; CC(C)(C)OC(=O)[C@@H]1CC(=O)CN1C(=O)OCc1ccccc1]

### Synthesis of S2a

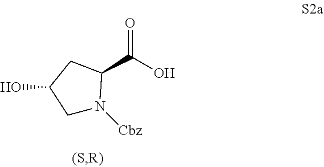 [SMILES: O=C(O)[C@@H]1C[C@@H](O)CN1C(=O)OCc1ccccc1]

### (2S,4R)-1-((benzyloxy)carbonyl)-4-hydroxypyrrolidine-2-carboxylic acid (S2a)—Purified Yield: 27.7 kg, 91%

Five reactions were carried out in parallel. To a solution of compound 1a (3.00 kg, 22.8 mol) and NaHCO3 (4.80 kg, 57.2 mol) in H2O (18.0 L) was added CbzCl (3.71 kg, 21.7 mol) in THF (3 L) and the mixture was stirred at 20° C. for 1 h. TLC (petroleum ether/ethyl acetate=3/1, R_{f}=0.00) showed CbzCl was consumed completely, TLC (dichloromethane/methanol=5/1, R_{f}=0.20) showed the product was detected. Five reactions were combined for workup. The reaction mixture was cooled to 0° C., and added dropwise HCl (12 M, 600.0 mL, pH=2). Then the mixture was extracted with EtOAc (8.00 L×2). The organic portion was washed with brine (5.00 L) and dried over Na₂SO₄. The mixture was concentrated in vacuo to give compound S2a as yellow oil. ¹H NMR from WuXi Apptec (400 MHz, DMSO, rotamers observed) δ 7.29-7.38 (m, 5H), 5.04-5.12 (m, 2H), 4.21-4.30 (m, 2H), 3.36-3.47 (m, 2H), 2.12-2.20 (m, 1H), 1.88-1.98 (m, 1H).

### Synthesis of S3a

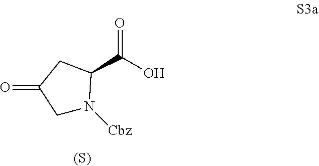

### (S)-1-((benzyloxy)carbonyl)-4-oxopyrrolidine-2-carboxylic acid (S3a)—Purified Yield: 15.0 kg, 92%

Five reactions were carried out in parallel. To a solution of compound S2a (5.00 kg, 18.8 mol) in EtOAc (25.0 L) was added TEMPO (148.0 g, 942 mmol) and trichloroisocyanuric acid (3.07 kg, 13.1 mol) at 0° C. The mixture was stirred at 0° C. for 3 h. TLC (dichloromethane/methanol=10/1, R_{f}=0.4) showed the starting material was consumed completely. Five reactions were combined for workup. The reaction mixture was filtered to remove the insoluble. The mixture was quenched by addition Na₂S₂O₃ (20.0 L) at 0-10° C. The combined organic layers were washed with brine (10.0 L×2), and dried over Na₂SO₄, filtered, and concentrated under reduced pressure to give a residue. The crude product was triturated with Petroleum ether/MTBE=1/1 (10.0 L) at 25° C. for 1 h, filtered and the solvent was evaporated to provide the compound S3a as a white solid. ¹H NMR from WuXi Apptec (400 MHZ, CDCl₃, rotamers observed) δ 9.05-9.09 (m, 1H), 7.26-7.37 (m, 5H), 5.16-5.27 (m, 2H), 4.86-4.92 (m, 1H), 3.89-4.02 (m, 2H), 2.94-3.06 (m, 1H), 2.67-2.77 (m, 1H).

### Synthesis of 2a

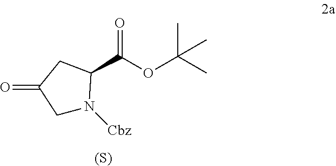

### 1-benzyl-2-(tert-butyl)(S)-4-oxopyrrolidine-1,2-dicarboxylate (2a)—Purified Yield: 10.0 kg, 69%

Four reactions were carried out in parallel. To a solution of compound S3a (3.00 kg, 11.4 mol) in THF (12.0 L) and cyclohexane (12.0 L) was added tert-butyl 2,2,2-trichloroacetimidate (4.98 kg, 22.7 mol) at 12° C. The mixture was added dropwise diethyloxonio(trifluoro) boranuide (161.0 g, 1.14 mol) at 0° C. The mixture was stirred at 12° C. for 2 h. TLC (dichloromethane/methanol=10/1, R_{f}=0.8) showed the starting material was consumed completely. Four reactions were combined for workup. The mixture was quenched by NaHCO₃ (4.00 L), adjusted to pH=8, then washed with brine (5.00 L), and dried over Na₂SO₄. The organic layer was concentrated to give residue. The residue was filtered to remove the insoluble material, and the solution was concentrated in vacuo to give compound 2a as a brown oil. ¹H NMR from WuXi Apptec (400 MHz, CDCl₃, rotamers observed) δ 7.32-7.38 (m, 5H), 5.12-5.26 (m, 2H), 4.68-4.76 (m, 1H), 3.88-4.02 (m, 2H), 2.90-3.00 (m, 1H), 2.52-2.58 (m, 1H), 1.41 (s, 9H, rotameric). HRMS (ESI-TOF) m/z: [M+Na]⁺ Calcd for C₁₇H₂₁NO₅Na 342.1312; found 342.1311.

### Synthesis of 2b at Multi-Kilogram Scale

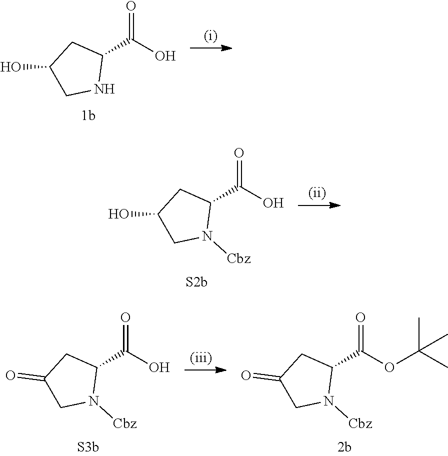 [SMILES: O=C(O)[C@H]1C[C@@H](O)CN1 ; O=C(O)[C@H]1C[C@@H](O)CN1C(=O)OCc1ccccc1 ; O=C1C[C@H](C(=O)O)N(C(=O)OCc2ccccc2)C1 ; CC(C)(C)OC(=O)[C@H]1CC(=O)CN1C(=O)OCc1ccccc1]

### Synthesis of S2b

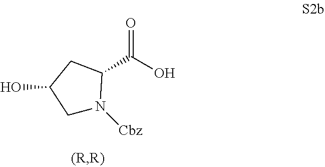 [SMILES: O=C(O)[C@H]1C[C@@H](O)CN1C(=O)OCc1ccccc1]

### (2R,4R)-1-((benzyloxy)carbonyl)-4-hydroxypyrrolidine-2-carboxylic acid (S2b)—Purified Yield: 15.0 kg, 82%

Three reactions were carried out in parallel. To a solution of compound 1b (3.00 kg, 22.8 mol) and NaHCO₃ (4.80 kg, 57.2 mol) in H₂O (18.0 L) was added CbzCl (3.71 kg, 21.7 mol) in THF (3.00 L) and the mixture was stirred at 20° C. for 3 h. TLC (petroleum ether/ethyl acetate=3/1, R_{f}=0.00) showed CbzCl was consumed completely, TLC (dichloromethane/methanol=5/1, R_{f}=0.30) showed the product was detected. Three reactions were combined for workup. The reaction mixture was cooled to 0° C., and HCl was added dropwise (12 M, 600 mL, until pH=2). Then the mixture was extracted with EtOAc (8.00 L×2). The organic portions were washed with brine (5.00 L) and dried over Na₂SO₄. The mixture was concentrated in vacuum to give compound S2b as a white solid. ¹H NMR from WuXi Apptec (400 MHZ, DMSO-d₆, rotamers observed) δ 7.30-7.37 (m, 5H), 5.02-5.08 (m, 2H), 4.19-4.28 (m, 2H), 3.55-3.59 (m, 1H), 3.19-3.23 (m, 1H), 2.32-2.40 (m, 1H), 1.89-1.93 (m, 1H).

### Synthesis of S3b

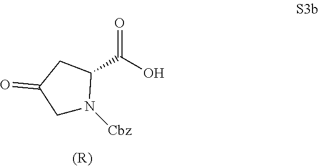

### (R)-1-((benzyloxy)carbonyl)-4-oxopyrrolidine-2-carboxylic acid (S3b)—Purified Yield: 12.0 kg, 94%

Three reactions were carried out in parallel. To a solution of compound S2b (4.30 kg, 16.21 mol) in DCM (28.0 L) was added TEMPO (127.5 g, 810.5 mmol) and trichloroisocyanuric acid (2.64 kg, 11.4 mol) at 0° C. The mixture was stirred at 0° C. for 3 h. TLC (dichloromethane/methanol=10/1, R_{f}=0.40) showed the starting material was consumed completely. Three reactions were combined for workup. The reaction mixture was filtered to remove the insoluble. The mixture was quenched by addition Na₂S₂O₃ (20.0 L) at 0-10° C. The combined organic layers were washed with brine (10.0 L×2), and dried over Na₂SO₄, filtered, and concentrated under reduced pressure to give a residue. The crude product was triturated with Petroleum ether/MTBE=1/1 (10.0 L) at 25° C. for 1 h, filtered and the solvent was evaporated to provide compound S3b as a white solid. ¹H NMR from WuXi Apptec (400 MHZ, CDCl₃, rotamers observed) δ 9.15 (br, 1H), 7.32-7.35 (m, 5H), 5.15-5.28 (m, 2H), 4.84-4.90 (m, 1H), 3.88-4.00 (m, 2H), 2.92-2.99 (m, 1H), 2.70-2.74 (m, 1H).

### Synthesis of 2b

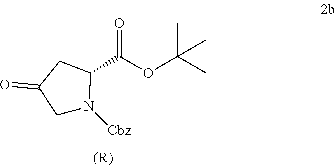

### 1-benzyl-2-(tert-butyl)(R)-4-oxopyrrolidine-1,2-dicarboxylate (2b)—Purified Yield: 13.0 kg, 89%

Four reactions were carried out in parallel. To a solution of compound S3b (3.00 kg, 11.4 mol) in THF (12.0 L) and cyclohexane (12.0 L) was added tert-butyl 2,2,2-trichloroacetimidate (4.98 kg, 22.7 mol) at 12° C. To the mixture was added dropwise diethyloxonio(trifluoro) boranuide (161.0 g, 1.14 mol) at 0° C. The mixture was stirred at 12° C. for 2 h. TLC (dichloromethane/methanol=10/1, R_{f}=1.00) showed the starting material was consumed completely. TLC (Petroleum ether/Ethyl acetate=3/1, R_{f}=0.5) showed the product was detected. Four reactions were combined for workup. The mixture was quenched by NaHCO₃ (4.00 L), adjusted to pH=8, then washed with brine (5.00 L), and dried over Na₂SO₄. The organic layer was concentrated to give residue. The residue was filtered to remove the insoluble and concentrated in vacuo to give compound 2b as brown oil. ¹H NMR from WuXi Apptec (400 MHz, CDCl₃, rotamers observed) δ 7.32-7.36 (m, 5H), 5.13-5.24 (m, 2H), 4.69-4.75 (m, 1H), 3.90-4.02 (m, 2H), 2.90-2.98 (m, 1H), 2.52-2.57 (m, 1H), 1.40 (s, 9H, rotameric). HRMS (ESI-TOF) m/z: [M+Na]⁺ Calcd for C₁₇H₂₁NO₅Na 342.1312; found 342.1312.

### Synthesis of Compounds 4a-d

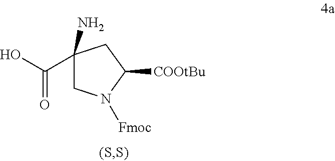

### (3S,5S)-1-(((9H-fluoren-9-yl)methoxy)carbonyl)-3-amino-5-(tert-butoxycarbonyl)pyrrolidine-3-carboxylic acid (4a)—Purified Yield, 12.2 g, 71%

Compound 3a-d (38 mmol) was dissolved in Na₂CO₃ (300 mL, 0.2 mol/L). To this solution, Pd/C (10% wt) was added, and an H₂-filled balloon was attached by a 3-way valve. The atmosphere was removed by vacuum evacuation and back filled of H₂ several times. After stirring at room temperature overnight, completion of the reaction was reached (monitored by LCMS). To the stirring solution, CuCl₂ (20 mmol) was added and reacted for 30 minutes. Fmoc-Cl (40 mmol) dissolved in EtOAc (100 mL) was added dropwise to the reaction mixture and stirring vigorously for another 2 hours. Pd/C was removed by vacuum filtration using celite and organic layer was collected, washed with saturated NaCl solution (250 mL×2) and transferred to a beaker. The solution was then mixed with EDTA sodium salt solution (250 mL, 0.2M), stirring vigorously for overnight. The product 4a-d was separated under vacuum filtration as white solid. This four-step sequence of reactions is performed without any chromatography. ¹H NMR (500 MHZ, DMSO) δ 8.78 (s, 2H), 7.91 (t, J=7.4 Hz, 2H), 7.64 (d, J=7.5 Hz, 2H, rotameric), 7.43 (t, J=7.3 Hz, 2H), 7.34-7.31 (m, 2H), 4.42-4.15 (m, 4H, rotameric), 4.04 (d, J=11.5 Hz, 1H, rotameric), 3.58 (d, J=11.5 Hz, 1H, rotameric), 2.88 (dd, J=13.4, 8.5 Hz, 1H, rotameric), 2.21 (dd, J=13.4, 8.8 Hz, 1H, rotameric), 1.40 (s, 9H). ¹³C NMR (125 MHz DMSO, rotamers observed) δ 172.1, 171.7, 170.8, 170.7, 154.1, 154.0, 144.2, 144.1, 144.0, 143.7, 141.2, 128.3, 128.2, 127.6, 125.9, 125.7, 125.6, 120.6, 81.9, 81.5, 67.7, 67.3, 62.0, 61.0, 60.2, 59.9, 55.1, 54.3, 47.1, 46.9, 38.9, 37.9, 28.1, 28.00; HRMS (ESI-TOF) m/z: [M+H]⁺ Calcd for C₂₅H₂₉N₂O₆ 453.2020; found 453.2016.

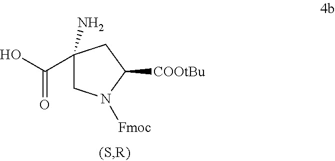

### (3R,5S)-1-(((9H-fluoren-9-yl)methoxy)carbonyl)-3-amino-5-(tert-butoxycarbonyl)pyrrolidine-3-carboxylic acid (4b)—Purified Yield, 13.0 g, 74%

¹H NMR (500 MHZ, DMSO) δ 8.74 (s, 2H), 7.91 (d, J=7.5 Hz, 2H), 7.72-7.66 (m, 2H), 7.44 (t, J=7.4 Hz, 2H), 7.36-7.31 (m, 2H), 4.61, 4.41 (m, 1H, rotameric), 4.40-4.04 (m, 2H, rotameric), 3.99 (m, 1H, rotameric), 3.85, 3.76 (d, J=12.2 Hz, 1H, rotameric), 2.77, 2.67 (dd, J=14.1, 9.0 Hz, 1H, rotameric), 2.50 (dd, J=14.3, 7.8 Hz, 1H, rotameric), 1.40 (d, J=5.3 Hz, 9H); ¹³C NMR (125 MHz, DMSO, rotamers observed) δ 170.9, 170.3, 169.9, 163.6, 154.0, 153.8, 144.1, 144.0, 143.7, 141.2, 141.1, 128.3, 128.2, 127.7, 127.6, 125.9, 125.8, 125.7, 120.7, 120.6, 82.2, 81.8, 68.0, 67.7, 63.9, 63.0, 58.9, 58.4, 55.0, 54.4, 47.0, 46.9, 38.1, 28.0; HRMS (ESI-TOF) m/z: [M+H]⁺ Calcd for C₂₅H₂₉N₂O₆ 453.2020, found 453.2028.

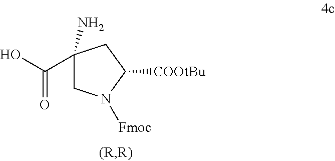

### (3R,5R)-1-(((9H-fluoren-9-yl)methoxy)carbonyl)-3-amino-5-(tert-butoxycarbonyl)pyrrolidine-3-carboxylic acid (4c)—Purified Yield, 10.9 g, 64%

¹H NMR (500 MHZ, DMSO) δ 8.78 (s, 2H), 7.91 (t, J=7.4 Hz, 2H), 7.64 (d, J=7.5 Hz, 2H, rotameric), 7.43 (t, J=7.3 Hz, 2H), 7.34-7.31 (m, 2H), 4.42-4.15 (m, 4H, rotameric), 4.04 (d, J=11.4 Hz, 1H, rotameric), 3.58 (d, J=11.5 Hz, 1H, rotameric), 2.88, 2.84 (dd, J=13.3, 8.5 Hz, 1H, rotameric), 2.24, 2.21 (dd, J=13.3, 8.8 Hz, 1H, rotameric), 1.41 (s, 9H); ¹³C NMR (125 MHz, DMSO, rotamers observed) δ 172.1, 171.7, 170.9, 170.8, 154.1, 154.0, 144.2, 144.1, 144.0, 143.7, 141.1, 128.3, 128.2, 127.6, 125.9, 125.7, 125.6, 121.9, 120.6, 120.5, 81.9, 81.5, 67.7, 67.3, 62.0, 61.0, 60.2, 59.9, 55.1, 54.3, 47.1, 46.9, 38.9, 37.9, 28.1, 28.0; HRMS (ESI-TOF) m/z: [M+H]⁺ Calcd for C₂₅H₂₉N₂O₆ 453.2020, found 453.2017.

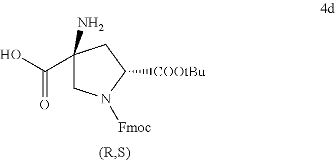

### (3R,5S)-1-(((9H-fluoren-9-yl)methoxy)carbonyl)-3-amino-5-(tert-butoxycarbonyl)pyrrolidine-3-carboxylic acid (4d)—Purified Yield, 14.7 g, 85%

¹H NMR (500 MHZ, DMSO) δ 8.75 (s, 2H), 7.91 (d, J=7.6 Hz, 2H), 7.72-7.66 (m, 2H), 7.44 (t, J=7.5 Hz, 2H), 7.36-7.31 (m, 2H), 4.61, 4.41 (m, 1H, rotameric), 4.40-4.04 (m, 2H, rotameric), 3.99 (m, 1H, rotameric), 3.85, 3.78 (d, J=12.2 Hz, 1H, rotameric), 2.77, 2.67 (dd, J=14.1, 7.7 Hz, 1H, rotameric), 2.53, 2.45 (dd, J=14.3, 7.6 Hz, 1H, rotameric), 1.40 (d, J=5.3 Hz, 9H); ¹³C NMR (125 MHz, DMSO, rotamers observed) δ 170.8, 170.3, 169.9, 163.6, 154.0, 153.8, 144.1, 143.7, 141.2, 141.1, 128.4, 128.2, 127.7, 127.6, 125.9, 125.8, 125.7, 120.7, 82.2, 81.8, 68.0, 67.7, 63.8, 62.9, 58.8, 58.4, 54.9, 54.6, 47.0, 38.1, 28.0; HRMS (ESI-TOF) m/z: [M+H]⁺ Calcd for C₂₅H₂₉N₂O₆ 453.2020, found 453.2029.

### Synthesis of Compounds 5a-d

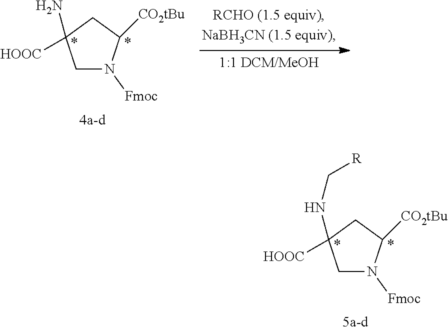

### Synthesis of Functionalized Bis-Amino Acids 5a

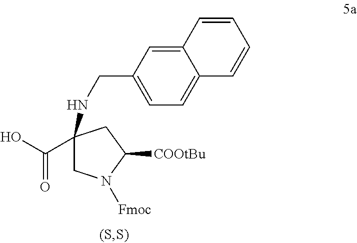

### (3S,5S)-1-(((9H-fluoren-9-yl)methoxy)carbonyl)-5-(tert-butoxycarbonyl)-3-((naphthalen-2-ylmethyl)amino) pyrrolidine-3-carboxylic acid (5a)—Purified Yield, 1.82 g, 61%

2-naphthaldehyde (7.5 mmol) was added to a 100 mL round bottom flask containing compound 4a (5 mmol) which was dissolved in 25 mL MeOH. Stirring the solution for 30 min at which time NaBH₃CN (7.5 mmol) was added and allowed to stir overnight. Reaction progress was determined by LCMS and, if incomplete, additional aldehyde and reducing agent was added. When the reductive alkylation was completed, the solution was transferred to a round bottom flask containing 10 g Celite. Solvent was removed under reduced pressure and the dry powder was then transferred into a loading cartridge for flash chromatography. Normal phase separation was performed with a mobile phase of DCM/20% MeOH in DCM using a 0-100% gradient. Fractions containing pure product was collected and the solvent was removed under reduced pressure to yield functionalized bis-amino acid 5a. The purity was checked by LCMS. ¹H NMR (500 MHz, DMSO) δ 7.90-7.83 (m, 6H), 7.68-7.63 (m, 2H), 7.53-7.38 (m, 5H), 7.34-7.28 (m, 2H), 4.39, 4.32 (m, 1H, rotameric), 4.23 (m, 2H, rotameric), 4.15 (m, 1H, rotameric), 4.00, 3.90 (m, 1H, rotameric), 3.88 (m, 1H), 3.82 (m, 1H), 3.52, 3.46 (d, J=10.8 Hz, 1H, rotameric), 2.76-2.68 (m, 1H, rotameric), 2.15, 2.07 (dd, J=12.8, 6.0 Hz, 1H, rotameric), 1.36 (s, 9H); ¹³C NMR (125 MHz, DMSO) δ 174.4, 171.2, 170.8, 154.3, 144.3, 144.1, 143.9, 141.1, 137.2, 133.3, 132.7, 128.2, 128.0, 127.6, 127.4, 127.0, 126.5, 126.1, 125.8, 125.7, 125.6, 120.6, 81.3, 81.0, 68.2, 67.5, 67.3, 59.7, 59.3, 54.9, 54.4, 49.0, 47.2, 46.9, 39.1, 38.0, 28.1; HRMS (ESI-TOF) m/z: [M+H]⁺ Calcd for C₃₋₆H₃₇N₂O₆ 593.2624, found 593.2638.

### Synthesis of Functionalized Bis-Amino Acids 5b

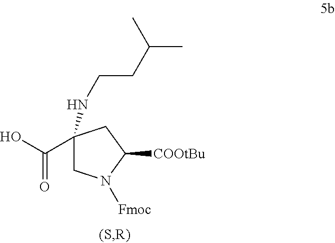

### (3R,5S)-1-(((9H-fluoren-9-yl)methoxy)carbonyl)-5-(tert-butoxycarbonyl)-3-(isopentylamino) pyrrolidine-3-carboxylic acid (5b)—Purified Yield, 2.34 g, 90%

Isovaleraldehyde (7.5 mmol) was added to a 100 mL round bottom flask containing compound 4b (5 mmol) which was suspended in 25 mL MeOH/DCM. Stirring the solution for 30 min at which time NaBH₃CN (7.5 mmol) was added and stirred overnight. Reaction progress was determined by LCMS and, if incomplete, additional aldehyde and reducing agent was added. When the reductive alkylation was completed, the solution was transferred to a round bottom flask containing 10 g Celite. Solvent was removed under reduced pressure and the dry powder was then transferred into a loading cartridge for flash chromatography. Normal phase separation was performed with a mobile phase of DCM/20% MeOH in DCM using a 0-100% gradient. Fractions containing pure product was collected and the solvent was removed under reduced pressure to yield functionalized bis-amino acid 5b. The purity was checked by LCMS. ¹H NMR (500 MHz, DMSO) δ 7.91-7.89 (m, 2H), 7.68-7.64 (m, 2H), 7.44-7.41 (m, 2H), 7.35-7.30 (m, 2H), 4.55, 4.49 (dd, J=9.1, 5.6 Hz, 1H, rotameric), 4.47-4.45 (m, 1H), 4.27, 4.23 (m, 1H, rotameric), 4.15 (m, 1H, rotameric), 4.05 (t, J=13.6 Hz, 1H), 3.84 (d, J=12.7 Hz, 1H, rotameric), 3.07-2.83 (m, 3H), 2.63, 2.55 (dd, J=14.8, 5.4 Hz, 1H, rotameric), 1.67-1.61 (m, 1H), 1.53-1.49 (m, 2H), 1.40 (s, 9H, rotameric), 0.89 (d, J=6.6 Hz, 6H); ¹³C NMR (125 MHZ, DMSO) δ 170.2, 169.9, 169.4, 169.3, 153.8, 153.7, 144.1, 143.7, 141.2, 128.3, 127.6, 125.7, 125.5, 120.6, 119.2, 116.9, 114.6, 112.3, 82.3, 81.9, 69.0, 68.1, 67.9, 58.9, 58.5, 53.5, 52.9, 47.0, 46.9, 43.0, 36.6, 35.3, 27.9, 25.7, 22.3; HRMS (ESI-TOF) m/z: [M+H]⁺ Calcd for C₃₀H₃₉N₂O₆ 523.2803, found 523.2797.

### Synthesis of Functionalized Bis-Amino Acids 5c

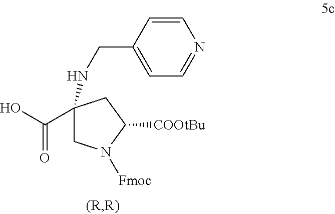

### (3R,5R)-1-(((9H-fluoren-9-yl)methoxy)carbonyl)-5-(tert-butoxycarbonyl)-3-((pyridin-4-ylmethyl)amino) pyrrolidine-3-carboxylic acid (5c)—Purified Yield, 2.16 g, 79%

4-Pyridinecarboxaldehyde (7.5 mmol) was added to a 100 mL round bottom flask containing compound 4c (5 mmol) which was dissolved in 25 mL MeOH. Stirring the solution for 30 min at which time NaBH₃CN (7.5 mmol) was added and allowed to proceed overnight. Reaction progress was determined by LCMS and, if incomplete, additional aldehyde and reducing agent was added. When the reductive alkylation was completed, the solution was transferred to a round bottom flask containing 10 g Celite. Solvent was removed under reduced pressure and the dry powder was then transferred into a loading cartridge for flash chromatography. Normal phase separation was performed with a mobile phase of DCM/20% MeOH in DCM using a 0-100% gradient. Fractions containing pure product was collected and the solvent was removed under reduced pressure to yield functionalized bis-amino acid 5c. The purity was checked by LCMS. ¹H NMR (500 MHz, DMSO) δ 8.46-8.43 (m, 2H), 7.89 (d, J=7.6 Hz, 2H), 7.69-7.64 (m, 2H), 7.43-7.39 (m, 2H), 7.36-7.30 (m, 4H), 4.37-4.04 (m, 4H), 3.96, 3.85 (d, J=10.2 Hz, 1H, rotameric), 3.63 (q, J=13.9 Hz, 2H), 3.32, 3.24 (d, J=10.2 Hz, 1H, rotameric), 2.64-2.55 (m, 1H, rotameric), 1.93, 1.86 (dd, J=12.3, 5.9 Hz, 1H, rotameric), 1.35 (s, 9H, rotameric); ¹³C NMR (125 MHz, DMSO) δ 176.3, 171.7, 171.3, 154.4, 154.3, 151.1, 149.6, 144.5, 144.2, 143.9, 141.1, 128.2, 127.6, 125.8, 125.7, 125.6, 123.4, 120.6, 80.8, 80.5, 69.0, 68.0, 67.3, 67.1, 60.0, 59.7, 55.8, 55.2, 48.1, 47.2, 47.0, 39.1, 28.1; HRMS (ESI-TOF) m/z: [M+H]⁺ Calcd for C₃₁H₃₄N₃O₆ 544.2442, found 544.2445.

### Synthesis of Functionalized Bis-Amino Acids 5d

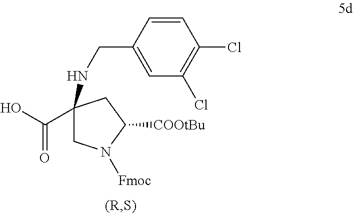

### (3S,5R)-1-(((9H-fluoren-9-yl)methoxy)carbonyl)-5-(tert-butoxycarbonyl)-3-((3,4-dichlorobenzyl)amino) pyrrolidine-3-carboxylic acid (5d)—Purified Yield, 2.67 g, 87%

3,4-Dichlorobenzaldehyde (7.5 mmol) was added to a 100 mL round bottom flask containing compound 4d (5 mmol) which was suspended in 25 mL MeOH. Stirring the solution for 30 min at which time NaBH₃CN (7.5 mmol) was added and allowed to proceed overnight. Reaction progress was determined by LCMS and, if incomplete, additional aldehyde and reducing agent was added. When the reductive alkylation was completed, the solution was transferred to a round bottom flask containing 10 g Celite. Solvent was removed under reduced pressure and the dry powder was then transferred into a loading cartridge for flash chromatography. Normal phase separation was performed with a mobile phase of DCM/20% MeOH in DCM using a 0-100% gradient. Fractions containing pure product was collected and the solvent was removed under reduced pressure to yield functionalized bis-amino acid 5d. The purity was checked by LCMS. ¹H NMR (500 MHZ, DMSO) δ 7.91-7.86 (m, 2H), 7.68 (d, J=7.5 Hz, 1H, rotameric), 7.59-7.49 (m, 3H), 7.43-7.22 (m, 5H), 4.36-4.27 (m, 1H, rotameric), 4.22-4.11, 4.04 (m, 3H, rotameric), 3.73-3.45 (m, 4H), 2.34, 2.10 (m, 1H, rotameric), 2.21 (dd, J=12.8, 8.5 Hz, 1H, rotameric), 1.38 (s, 9H, rotameric); ¹³C NMR (125 MHz, DMSO) δ 174.8, 174.7, 172.1, 171.6, 154.5, 154.4, 144.3, 144.1, 143.7, 141.1, 131.2, 131.1, 130.6, 130.5, 130.1, 130.0, 129.4, 129.3, 128.5, 128.3, 128.1, 127.6, 127.5, 125.8, 125.6, 125.5, 125.4, 120.7, 120.6, 81.3, 80.9, 69.4, 68.6, 67.5, 67.0, 59.9, 59.4, 55.2, 54.2, 47.2, 47.1, 46.9, 38.9, 28.1, 28.0; HRMS (ESI-TOF) m/z: [M+H]⁺ Calcd for C₃₂H₃₃Cl₂N₂O₆ 611.1710, found 611.1714.

### Synthesis of Compounds 6a-d

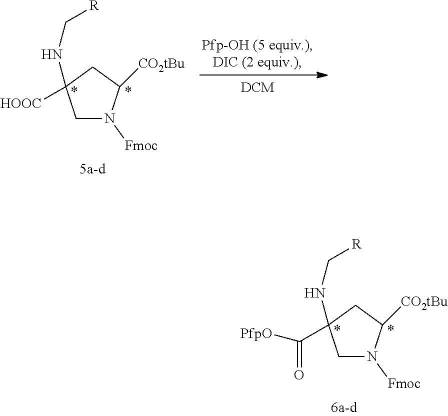

### Synthesis of Functionalized Pentafluorophenyl Ester 6a

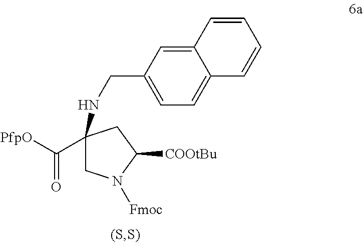

### 1-((9H-fluoren-9-yl)methyl)2-(tert-butyl)4-(perfluorophenyl)(2S,4S)-4-((naphthalen-2-ylmethyl)amino) pyrrolidine-1,2,4-tricarboxylate (6a)—Purified Yield, 1.07 g, 71%

Compound 5a (2 mmol) and pentafluorophenol (10 mmol) was weighted and dissolved in mL of DCM. DIC (4 mmol) was added, and the reaction was left to stir overnight. The completion of the reaction s checked by LCMS, and the reaction was then transferred to a 50 mL round bottom flask containing Celite (5 g). At room temperature, white powder was obtained by solvent removal under reduced pressure. Loading the cartridge with the dry Celite to run the flash chromatography using hexane/EtOAc using a 5-100% EtOAc gradient. Fractions containing pure product were collected under reduced pressure to yield 6a as a yellowish solid. ¹H NMR (500 MHz, CDCl₃, rotamers observed) δ 7.83-7.74 (m, 6H), 7.64-7.57 (m, 2H), 7.51-7.46 (m, 3H), 7.42-7.36 (m, 2H), 7.32-7.25 (m, 2H), 4.56-4.46 (m, 2H), 4.38-4.21 (m, 3H), 4.00-3.84 (m, 3H), 2.94 (m, 1H), 2.50 (dt, J=12.9, 4.7 Hz, 1H, rotameric), 1.43 (s, 9H, rotameric); ¹³C NMR (125 MHz, CDCl₃, rotamers observed) δ 170.2, 170.1, 169.7, 169.6, 154.4, 144.2, 144.0, 143.7, 143.5, 141.3, 141.2, 136.0, 135.9, 133.4, 132.9, 128.3, 127.8, 127.7, 127.1, 127.0, 126.9, 126.6, 126.5, 126.2, 125.9, 125.3, 125.2, 125.1, 125.0, 120.0, 82.2, 82.1, 68.8, 68.0, 67.9, 58.9, 58.7, 54.6, 54.2, 49.7, 49.0, 47.3, 47.1, 39.5, 38.3, 28.1, 28.0, 24.6; HRMS (ESI-TOF) m/z: [M+H]⁺ Calcd for C₄₂H₃₆F₅N₂O₆ 759.2423, found 759.2488.

### Synthesis of Functionalized Pentafluorophenyl Ester 6b

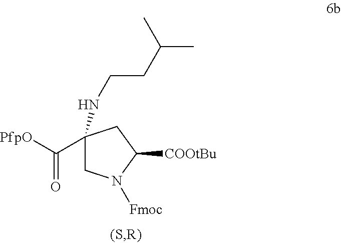

### 1-((9H-fluoren-9-yl)methyl)2-(tert-butyl)4-(perfluorophenyl)(2S,4R)-4-(isopentylamino) pyrrolidine-1,2,4-tricarboxylate (6b)—Purified Yield, 1.01 g, 73%

Compound 5b (2 mmol) and pentafluorophenol (10 mmol) was weighted and dissolved in 10 mL of DCM. DIC (4 mmol) was added and the left reaction to stir overnight. The completion of the reaction was checked by LCMS, and reaction was then transferred to a 50 mL round bottom flask containing Celite (5 g). At room temperature, white powder was obtained by solvent removal under reduced pressure. Loading the cartridge with the dry Celite to run the flash chromatography using hexane/EtOAc using a 5-100% EtOAc gradient. Fractions containing pure product were collected under reduced pressure to yield 6b (50.7%) as a white solid. ¹H NMR (500 MHz, CDCl₃, rotamers observed) δ 7.78-7.76 (m, 2H), 7.69-7.59 (m, 2H), 7.43-7.39 (m, 2H), 7.34-7.30 (m, 2H), 4.55-4.20 (m, 4H), 4.05 (t, J=13.6 Hz, 1H), 4.02, 3.84 (m, 1H, rotameric), 2.63-2.43 (m, 4H), 1.46 (s, 9H, rotameric); 1.41 (q, J=7.2 Hz, 2H), 1.22 (d, J=6.4 Hz, 1H), 0.92-0.89 (m, 6H); ¹³C NMR (125 MHz, CDCl₃, rotamers observed) δ 171.0, 170.7, 168.8, 168.7, 154.7, 144.2, 144.0, 143.8, 143.5, 141.4, 141.3, 127.8, 127.7, 127.2, 127.1, 125.5, 125.2, 125.1, 120.0, 82.2, 82.0, 68.2, 68.1, 67.7, 67.4, 59.2, 58.9, 54.6, 54.1, 47.2, 47.1, 42.7, 42.7, 39.4, 39.4, 38.9, 37.8, 28.0, 26.0, 25.9, 22.6, 22.5; HRMS (ESI-TOF) m/z: [M+H]⁺ Calcd for C₃₋₆H₃₈F₅N₂O₆ 689.2645, found 689.2649.

### Synthesis of Functionalized Pentafluorophenyl Ester 6c

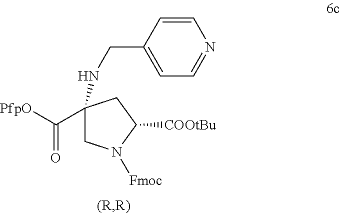

### 1-((9H-fluoren-9-yl)methyl)2-(tert-butyl)4-(perfluorophenyl)(2R,4R)-4-((pyridin-4-ylmethyl)amino) pyrrolidine-1,2,4-tricarboxylate (6c)—Purified Yield, 0.752 g, 53%

Compound 5c (2 mmol) and pentafluorophenol (10 mmol) was weighted and dissolved in 10 mL of DCM. DIC (4 mmol) was added, and the reaction was left to stir overnight. The completion of the reaction was checked by LCMS, and the reaction was then transferred to a 50 mL round bottom flask containing Celite (5 g). At room temperature, white powder was obtained by solvent removal under reduced pressure. Loading the cartridge with the dry Celite to run the flash chromatography using hexane/EtOAc using a 5-100% EtOAc gradient. Fractions containing pure product were collected under reduced pressure to yield 6c as a white solid. ¹H NMR (500 MHz, CDCl₃, rotamers observed) δ 8.58-8.45 (m, 2H), 7.78-7.75 (m, 2H), 7.64-7.56 (m, 2H), 7.42-7.37 (m, 4H), 7.32-7.25 (m, 2H), 4.55-4.47 (m, 2H), 4.41-4.33 (m, 1H), 4.29-4.10 (m, 2H), 3.89-3.74 (m, 3H), 2.96-2.86 (m, 1H, rotameric), 2.47 (td, J=13.8, 3.9 Hz, 1H), 1.44 (d, 9H, rotameric); ¹³C NMR (125 MHz, CDCl₃, rotamers observed) δ 175.6, 175.5, 171.1, 171.0, 170.2, 169.2, 169.1, 154.4, 149.5, 149.4, 148.5, 148.4, 147.6, 144.1, 143.9, 143.6, 143.4, 141.3, 127.8, 127.2, 127.1, 127.0, 125.2, 125.1, 125.0, 123.6, 123.5, 120.0, 82.5, 82.4, 68.7, 67.7, 58.8, 58.6, 54.4, 54.0, 48.1, 47.3, 47.1, 39.5, 38.2, 28.1; HRMS (ESI-TOF) m/z: [M+H]⁺ Calcd for C₃₇H₃₃F₅N₃O₆ 710.2284, found 710.2289.

### Synthesis of Functionalized Pentafluorophenyl Ester 6d

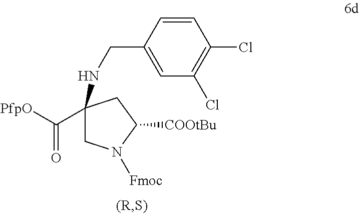

### 1-((9H-fluoren-9-yl)methyl)2-(tert-butyl)4-(perfluorophenyl)(2R,4S)-4-((3,4-dichlorobenzyl)amino) pyrrolidine-1,2,4-tricarboxylate (6d)—Purified Yield, 1.10 g, 71%

Compound 5d (2 mmol) and pentafluorophenol (10 mmol) was weighted and dissolved in 10 mL of DCM. DIC (4 mmol) was added, and the reaction was left to stir overnight. The completion of the reaction was checked by LCMS, and the reaction was then transferred to a 50 mL round bottom flask containing Celite (5 g). At room temperature, white powder was obtained by solvent removal under reduced pressure. Loading the cartridge with the dry Celite to run the flash chromatography using hexane/EtOAc using a 5-100% EtOAc gradient. Fractions containing pure product were collected under reduced pressure to yield 6d as a white solid. ¹H NMR (500 MHz, CDCl₃, rotamers observed) δ 7.78-7.51 (m, 4H), 7.45-7.14 (m, 7H), 4.58-4.45 (m, 2H, rotameric), 4.39-4.30 (m, 1H, rotameric), 4.22 (t, J=7.1 Hz, 1H), 4.10-4.02 (m, 1H), 3.97-3.64 (m, 3H), 2.63-2.46 (m, 2H), 1.46 (s, 9H, rotameric); ¹³C NMR (125 MHZ, CDCl₃, rotamers observed) δ 170.7, 170.5, 168.4, 168.3, 154.8, 154.6, 144.1, 143.9, 143.7, 143.4, 141.3, 141.3, 139.2, 139.1, 132.7, 131.6, 130.5, 129.9, 129.8, 127.8, 127.2, 127.1, 125.4, 125.1, 125.0, 124.9, 120.1, 120.0, 119.9, 82.5, 82.3, 68.2, 67.5, 59.1, 58.9, 54.3, 53.6, 47.7, 47.6, 47.2, 47.1, 39.3, 38.2, 28.0; HRMS (ESI-TOF) m/z: [M+H]⁺ Calcd for C₃₈H₃₂Cl₂F₅N₂O₆ 777.1481, found 777.1556.

### Solid Phase Synthesis of Spiroligomers

### General Procedures for Solid Phase Synthesis

### General Procedure for Resin Loading

Solid phase synthesis of Spiroligomers was performed on a microwave peptide synthesizer. Cl-TCP(Cl) Protide resin (100 mg, 0.05 mmol, 0.5 mmol/g loading) was weighed into a 25 mL reactor vessel. Fmoc-Pro-OH was weighed into a 2 mL centrifuge tube and dissolved in anhydrous DMF (2 mL) with DIPEA (168 μL, 1 mmol). This solution was then added, in one portion, to the resin in the reactor vessel and allowed to react for 30 min at 50° C. with Ar bubbling. After the solution was drained, the resin was washed with 2 mL DMF three times. With 2 mL capping solution containing 1:3:17 of methanol/DIPEA/DCM, the unreacted trityl chloride was capped for 10 min at room temperature and the resin was washed with DMF three times.

### General Procedure for Fmoc Deprotection

All deprotections were accomplished with 20% piperidine in DMF (2 mL) (1×4 min, 60° C.). After each Fmoc deprotection, the resin was washed with DMF (3×1 mL).

### General Procedure for Coupling of Building Blocks

The solution of building blocks (0.1 mmol, 2 equiv.) in 1 mL anhydrous DMF was added to the resin in the reactor vessel. Then, a solution of HOAt (27.2 mg, 0.2 mmol) and DIPEA (69.7 μL, 0.4 mmol) in 1 mL anhydrous DMF was added. The reaction mixture was heated for 1 hour at 50° C. The completion of reaction was confirmed by test cleavage with LCMS after washing with DMF.

### General Procedure for the Coupling of Fmoc-Dab (Boc)-OH

Fmoc-Dab (Boc)-OH (63.9 mg, 0.15 mmol, 3 equiv) in 1 mL DMF was transferred to the reactor together with a mixture of PyAOP (78.2 mg, 0.15 mmol), DIPEA (52.3 mL, 0.3 mmol) and DMF (2 mL) and they were left to react for 5 min at 50° C. The completion of reaction was checked by test cleavage with LCMS after washing with DMF.

### General Procedure for Cleavage and DKP Closure

Final cleavage was achieved by using TFA (2 mL×2) for 30 min at room temperature. The product was collected into a 50 mL centrifuge tube and transferred to a 20 mL glass vial. It was placed in a heating block with stirring at 40° C. overnight.

### Solid Phase Synthesis of T1

First residue Fmoc-Pro-OH (169 mg, 0.5 mmol) was loaded onto the resin (100 mg, 0.05 mmol, 0.5 mmol/g loading) with the general procedure for resin loading and deprotected with the general procedure for Fmoc deprotection. General procedure for coupling of building blocks and Fmoc deprotection were employed with 6a (75.8 mg, 0.1 mmol, 2 equiv.), followed by 6b (68.8 mg, 0.1 mmol), 6c (71 mg, 0.1 mmol), and 6d (77.3 mg, 0.1 mmol). Then, Fmoc-Dab (Boc)-OH (63.9 mg, 0.15 mmol) was coupled and deprotected as described above. Finally, the cleavage and DKP closure of T1 were performed by following the general procedure. After the completion of DKP closure confirmed by LCMS, the product was precipitated in cold diethyl ether and purified by preparative HPLC (25%-95% gradient of MeCN in water with 0.1% TFA). Purified fractions containing product were collected, and then lyophilized to yield the spiroligomer T1 as a white powder. T1—Purified Yield, 18 mg, 30%; HPLC purity, 98%; retention time, 3.122 min. HRMS (ESI-TOF) m/z: [M+H]⁺ Calcd for C₆₂H₆₇C₁₂N₁₂O₁₀ 1209.44, found 1209.44.

### Solid Phase Synthesis of T2

First residue Fmoc-Pro-OH (169 mg, 0.5 mmol) was loaded onto the resin (100 mg, 0.05 mmol, 0.5 mmol/g loading) with the general procedure for resin loading and deprotected with the general procedure for Fmoc deprotection. General procedure for coupling of building blocks and Fmoc deprotection were employed with 6d (77.3 mg, 0.1 mmol, 2 equiv.), followed by 6c (71 mg, 0.1 mmol), 6b (68.8 mg, 0.1 mmol), and 6a (75.8 mg, 0.1 mmol). Then, Fmoc-Dab (Boc)-OH (63.9 mg, 0.15 mmol) was coupled and deprotected as described above. Finally, the cleavage and DKP closure of T2 were performed by following the general procedure. After the completion of DKP closure confirmed by LCMS, the product was precipitated in cold diethyl ether and purified by preparative HPLC (25%-95% gradient of MeCN in water with 0.1% TFA). Purified fractions containing product were collected, and then lyophilized to yield the spiroligomer T2 as a white powder. T2—Purified Yield, 16 mg, 27%; HPLC purity, 96%; retention time, 3.297 min. HRMS (ESI-TOF) m/z: [M+H]⁺ Calcd for C₆₂H₆₆C₁₂N₁₂O₁₀ 1209.44, found 1209.45.

### Solid Phase Synthesis of T3

First residue Fmoc-Pro-OH (169 mg, 0.5 mmol) was loaded onto the resin (100 mg, 0.05 mmol, 0.5 mmol/g loading) with the general procedure for resin loading and deprotected with the general procedure for Fmoc deprotection. General procedure for coupling of building blocks and Fmoc deprotection were employed with 6b (68.8 mg, 0.1 mmol, 2 equiv.), followed by 6d (77.3 mg, 0.1 mmol), 6a (75.8 mg, 0.1 mmol), and 6c (71 mg, 0.1 mmol). Then, Fmoc-Dab (Boc)-OH (63.9 mg, 0.15 mmol) was coupled and deprotected as described above. Finally, the cleavage and DKP closure of T3 were performed by following the general procedure. After the completion of DKP closure confirmed by LCMS, the product was precipitated in cold diethyl ether and purified by preparative HPLC (25%-95% gradient of MeCN in water with 0.1% TFA). Purified fractions containing product were collected, and then lyophilized to yield the spiroligomer T3 as a white powder. T3—Purified Yield, 15 mg, 24%; HPLC purity, >99%; retention time, 3.515 min. HRMS (ESI-TOF) m/z: [M+H]⁺ Calcd for C₆₂H₆₇C₁₂N₁₂O₁₀ 1209.44, found 1209.45.

### Solid Phase Synthesis of T4

First residue Fmoc-Pro-OH (169 mg, 0.5 mmol) was loaded onto the resin (100 mg, 0.05 mmol, 0.5 mmol/g loading) with the general procedure for resin loading and deprotected with the general procedure for Fmoc deprotection. General procedure for coupling of building blocks and Fmoc deprotection were employed with 6c (71 mg, 0.1 mmol, 2 equiv.), followed by 6d (77.3 mg, 0.1 mmol), 6a (75.8 mg, 0.1 mmol), and 6b (68.8 mg, 0.1 mmol). Then, Fmoc-Dab (Boc)-OH (63.9 mg, 0.15 mmol) was coupled and deprotected as described above. Finally, the cleavage and DKP closure of T4 were performed by following the general procedure. After the completion of DKP closure confirmed by LCMS, the product was precipitated in cold diethyl ether and purified by preparative HPLC (25%-95% gradient of MeCN in water with 0.1% TFA). Purified fractions containing product were collected, and then lyophilized to yield the spiroligomer T4 as a white powder. T4—Purified Yield, 19 mg, 32%; HPLC purity, >99%; retention time, 3.333 min. HRMS (ESI-TOF) m/z: [M+H]⁺ Calcd for C₆₂H₆₇C₁₂N₁₂O₁₀ 1209.44, found 1209.45. (2S,4R)-1-((benzyloxy)carbonyl)-4-hydroxypyrrolidine-2-carboxylic acid (S2a).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A process comprising contacting a compound of Formula I with R¹X, R¹C(O)H, or R¹C(O)R²: 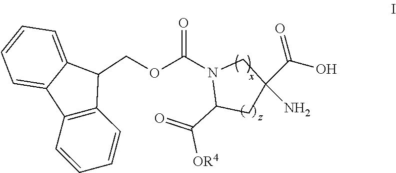
wherein:
R¹ is selected from the group consisting of an amino acid, β-amino acid, sugar, optionally substituted C₁₋₆ alkyl, optionally substituted C₂₋₆ alkenyl, optionally substituted C₂₋₆ alkynyl, optionally substituted aryl, and any combination thereof;
R² is absent or selected from the group consisting of H, amino acid, β-amino acid, sugar, optionally substituted C₁₋₆ alkyl, optionally substituted C₂₋₆ alkenyl, optionally substituted C₂₋₆ alkynyl, optionally substituted aryl, and any combination thereof;
or R¹ and R² are linked to form a C₃₋₈ cycloalkyl;
X is a leaving group;
R⁴ is a protecting group;
x is an integer 1 or 2; and
z is an integer 1 or 2;
with the proviso that both x and z are not 2;
in the presence of a reducing agent;
for a time and under conditions effective to produce a compound of Formula II: 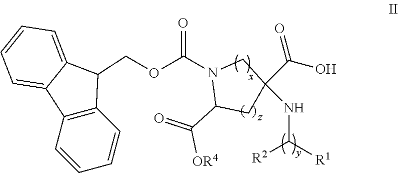
wherein:
y is an integer 0 or 1; with the proviso that when the compound of Formula I is reacted with R¹X, y is an integer 0 and R² is absent.

2. The process of claim 1, wherein R¹ and R² are, independently, substituted with C₀₋₆ alkylOH, C₀₋₆ alkylSH, C₀₋₆ alkylNH₂, C₀₋₆ alkyl-O—C₀₋₆ alkyl, C₀₋₆ alkyl-S—C₀₋₆ alkyl, C₀₋₆ alkylC(O)OH, C₀₋₆ alkylC(O)(C₁₋₆ alkyl), C₀₋₆ alkylC(O)O(C₁₋₆ alkyl), C₀₋₆ alkylN₃, C₀₋₆ alkylC(O)NH₂, C₀₋₆ alkyl-C(O)N(C₁₋₆ alkyl)OH, optionally substituted C₃₋₇ cycloalkyl, optionally substituted C₅₋₇ cycloalkenyl, optionally substituted aryl, optionally substituted heteroaryl, optionally substituted heterocyclyl, —NHC(NH₂)(═N(C₀₋₆ alkyl)), —NHC(NH₂)(═S(C₀₋₆ alkyl)), a nucleobase, or an amino acid;
wherein any carbon atom of said alkyl, alkenyl, or alkynyl is optionally replaced by a heteroatom that is O, S, SO, SO₂, or NR⁷;
R⁷ is selected from the group consisting of H, C₁₋₄ alkyl, C₃₋₄ alkenyl, C₃₋₄ alkynyl, C₁₋₄ bridging alkyl, and any combination thereof,
wherein a bridge is formed between the nitrogen and a carbon atom of said heteroatom-containing chain to form a ring,
said ring is optionally fused to Ar¹; and
Ar¹ is selected from the group consisting of optionally substituted C₃₋₆ cycloalkyl, heterocyclyl, aryl, heteroaryl, and any combination thereof.

3. The process of claim 2, wherein Ar¹ is selected from the group consisting of phenyl, 1-naphthyl, 2-naphthyl, indenyl, azulenyl, fluorenyl, and anthracenyl; or a heterocyclic aromatic group selected from the group consisting of 2-furyl, 3-furyl, 2-thienyl, 3-thienyl, 2-pyridyl, 3-pyridyl, 4-pyridyl, pyrrolyl, oxazolyl, thiazolyl, imidazolyl, pyrazolyl, 2-pyrazolinyl, pyrazolidinyl, isoxazolyl, isotriazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, benzo[b]thiophenyl, 1H-indazolyl, benzimidazolyl, benzthiazolyl, purinyl, 4 H-quinolizinyl, quinolinyl, 1,2,3,4-tetrahydroquinolinyl, isoquinolinyl, 1,2,3,4-tetrahydroisoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, and any combination thereof.

4. The process of claim 2, wherein:
Ar¹ is optionally substituted with one or more of H, halo, OH, NO₂, —SO₃H, CF₃, OCF₃, C₁₋₆ alkyl, C₂₋₆ alkenyl, C₁₋₆ alkoxy, O—C₃₋₄ alkenyl, —O-benzyl, —O-phenyl, 1,2-methylenedioxy, —NR⁵R⁶, —C(O)OH, —C(O)(C₁₋₆ alkyl), C(O)O(C₁₋₆ alkyl), —C(O)NH(C₁₋₆ alkyl), —C(O)NH(C₃₋₅ alkenyl), —C(O)N(C₁₋₆ alkyl)(C₁₋₆ alkyl), —C(O)(C₃₋₅ alkenyl)(C₃₋₆ alkenyl), morpholinyl, piperidinyl, —O—Ar², —CH₂—(CH₂)_{q}—Ar², —O—(CH₂)_{q}—Ar², —(CH₂)_{q}—O—Ar², or —CH═CH—Ar²;
wherein R⁵ and R⁶ are, independently, selected from the group consisting of H, C₁₋₆ alkyl, C₃₋₆ alkenyl, C₃₋₆ alkynyl, benzyl, and any combination thereof;
Ar² is selected from the group consisting of 4-methoxyphenyl, 2-pyridyl, 3-pyridyl, 4-pyridyl, pyrazyl, quinolyl, 3,5-dimethylisoxazoyl, 2-methylthiazoyl, thiazolyl, 2-thienyl, 3-thienyl, pyrimidinyl, and any combination thereof; and
q is an integer 0 to 2.

5. The process of claim 1, wherein one or both of R¹ and R² is selected from the group consisting of an amino acid, β-amino acid, sugar, or any combination thereof.

6. The process of claim 1, wherein R⁴ is a C₁₋₆ alkyl

7. The process of claim 1, wherein R⁴ is selected from the group consisting of t-butyl, 4-{N-[1-(4,4-dimethyl-2,6-dioxocyclohexylidene)-3-methylbutyl]amino}benzyl ester (DMab), and a combination thereof.

8. The process of claim 1, wherein X is selected from the group consisting of halogen and sulfonate.

9. The process of claim 1, wherein X is selected from the group consisting of chloro, fluoro, bromo, p-toluenesulfonate (OTs), methanesulfonate (OMs), and trifluoromethanesulfonate (OTf).

10. The process of claim 1, wherein the reducing agent is selected from the group consisting of NaBH₃, NaBH₃CN, and Na(CH₃COO)₃BH.

11. The process of claim 1, wherein the compound of Formula I has the structure of Formula I-A, I-B, or I-C:

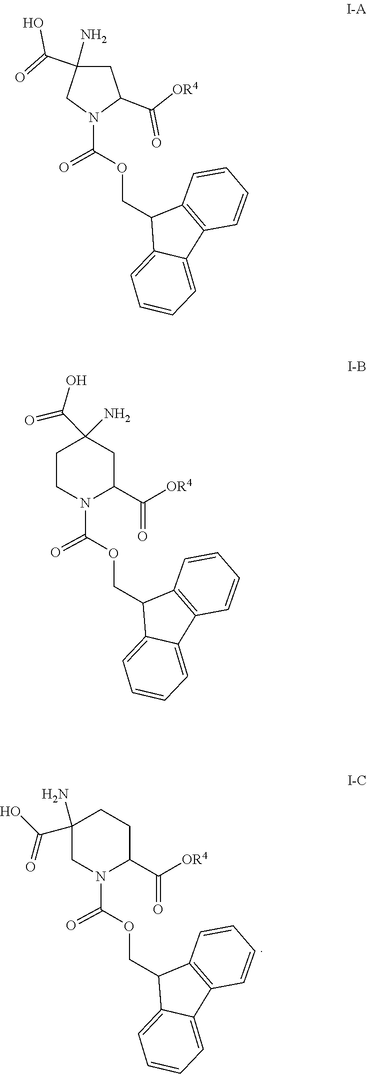

12. The process of claim 1, wherein the compound of Formula II has the structure of Formula II-A:

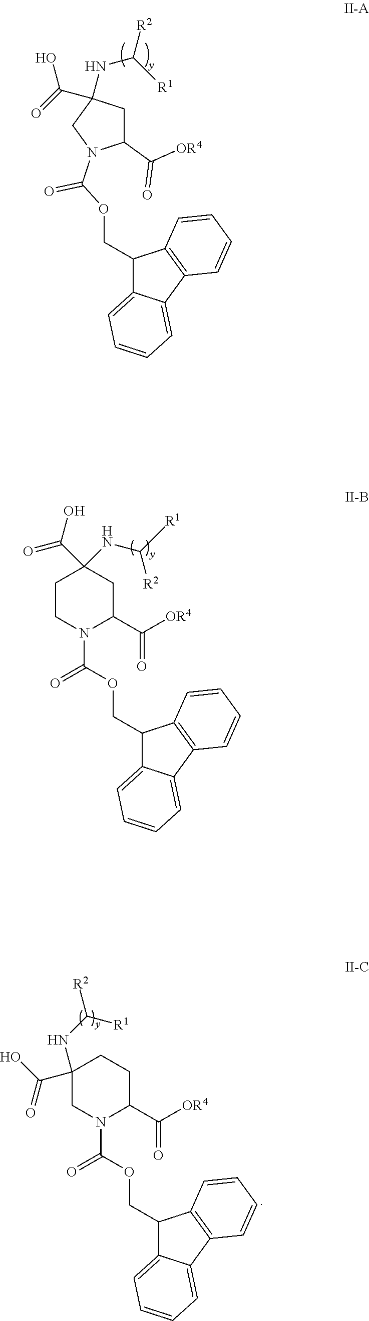

13. The process of claim 1, further comprising preparing the compound of Formula I by:
a) contacting a compound of Formula III: 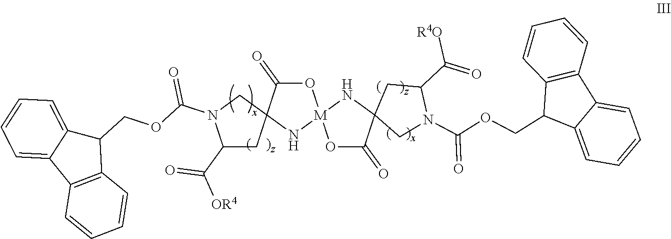
wherein M is a transition metal with a +2 oxidation state;
with a chelator;
for a time and under conditions effective to produce the compound of Formula I.

14. The process of claim 13, wherein M is selected from the group consisting of vanadium, manganese, iron, cobalt, nickel, copper, and zinc.

15. The process of claim 13, wherein the chelator is selected from the group consisting of ethylenediaminetetraacetic acid (EDTA) or a salt thereof, diethylenetriaminepentaacetic acid (DTPA) or a salt thereof, and hydroxyethylethylenediaminetriacetic acid (HEDTA) or a salt thereof.

16. The process of claim 15, wherein the salt of EDTA is selected from the group consisting of Na₂EDTA·H₂O and CaNa₂EDTA.

17. The process of claim 13, wherein the compound of Formula III is a compound having the structure of Formula III-A:

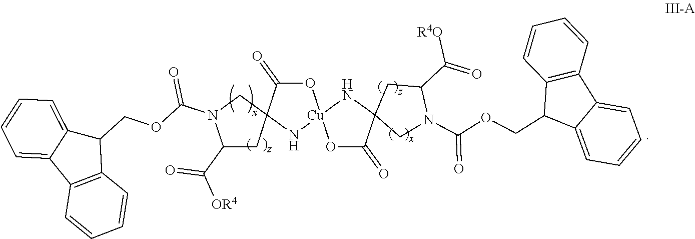

18. The process of claim 13, wherein the compound of Formula III is a compound having the structure of Formula III-B, III-C, III-D, III-E, III-F, or III-G:

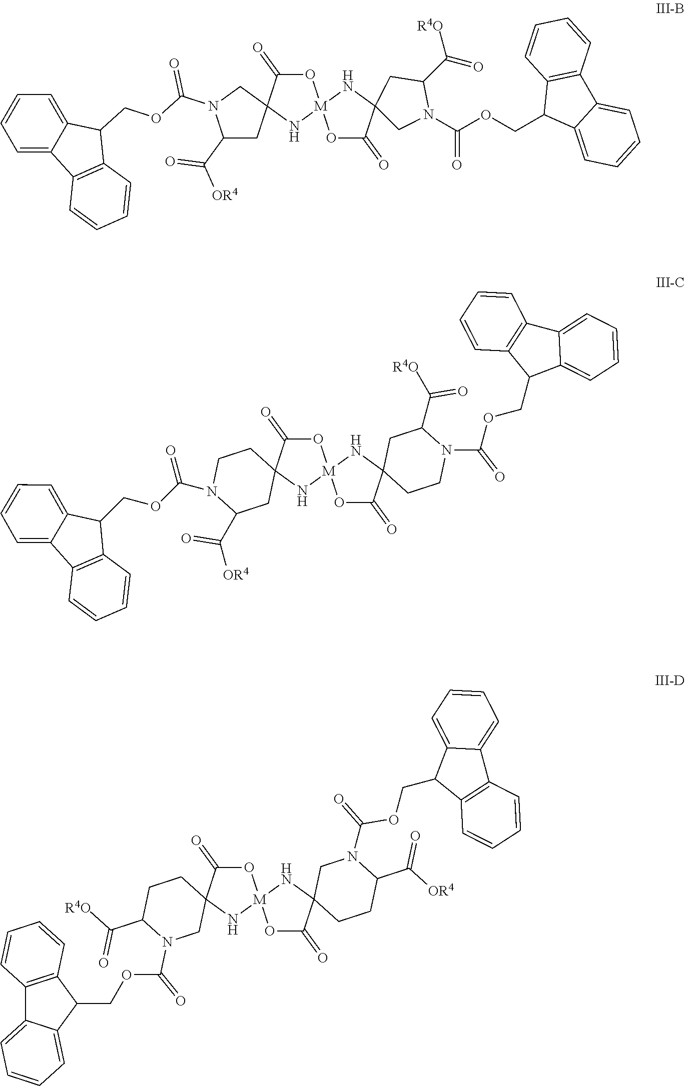

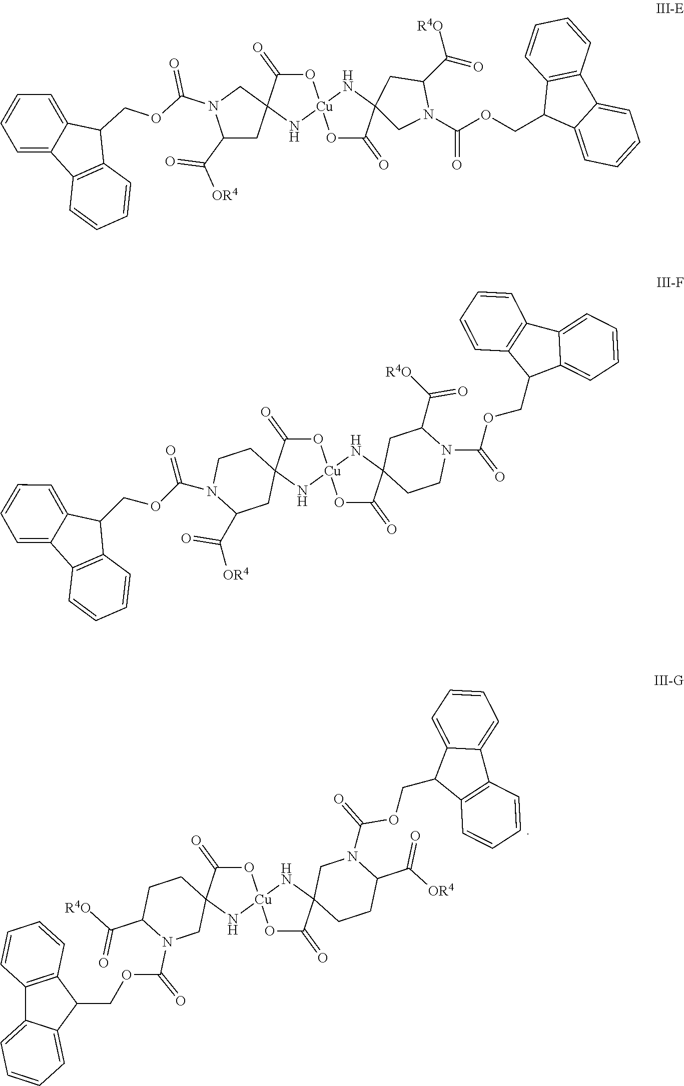

19. The process of claim 13, further comprising preparing the compound of Formula III by:
a) contacting a compound of Formula IV: 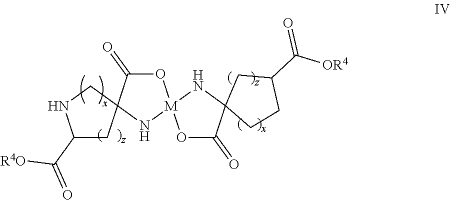
with 9-fluorenylmethoxycarbonyl chloride;
for a time and under conditions effective to produce the compound of Formula IV.

20. The process of claim 19, wherein the compound of Formula IV is a compound having the structure of Formula IV-A:

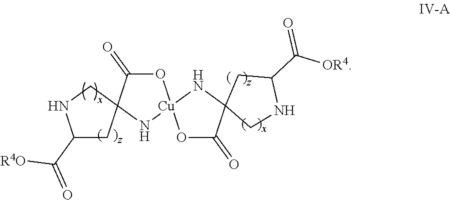

21. The process of claim 19, wherein the compound of Formula IV is a compound having the structure of Formula IV-B, IV-C, IV-D, IV-E, IV-F, or IV-G:

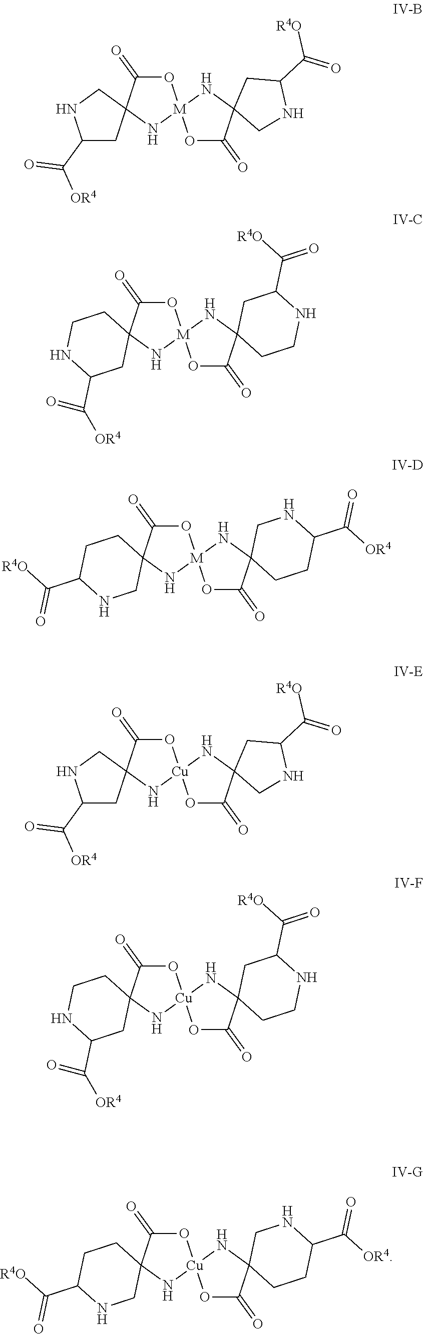

22. The process of claim 19, further comprising preparing the compound of Formula IV by:
a) contacting a compound of Formula V: 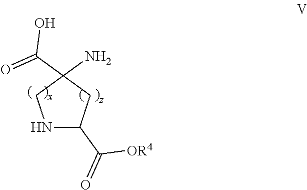
with a metal (II) source;
for a time and under conditions effective to produce the compound of Formula IV.

23. The process of claim 22, where the metal (II) source is selected from the group consisting of a Ni (II) source or a salt thereof and copper (II) source or a salt thereof.

24. The process of claim 22, wherein the metal (II) source is a copper (II) source or a salt thereof.

25. The process of claim 24, wherein the copper (II) salt is selected from the group consisting of copper chloride and copper sulfate.

26. The process of claim 22, wherein the compound of Formula V is a compound having the structure of Formula V-A, V-B, or V-C:

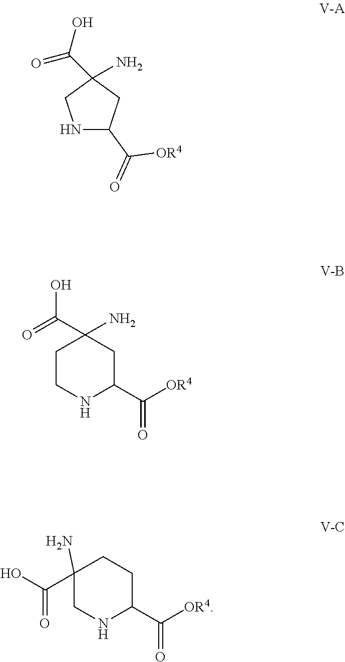

27. The process of claim 1, further comprising:
a) contacting the compound of Formula II with a compound of Formula VI-A, VI-B, or VI-C, with an activating agent and a base to provide an intermediate: 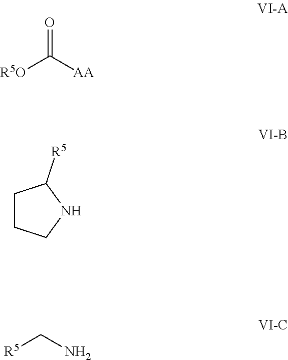
wherein AA is an optionally substituted amino acid; and
R⁵ is a resin; and
b) contacting the intermediate with a deprotectant for a time and under conditions effective to produce the compound of Formula VII-A, VII-B, or VII-C:

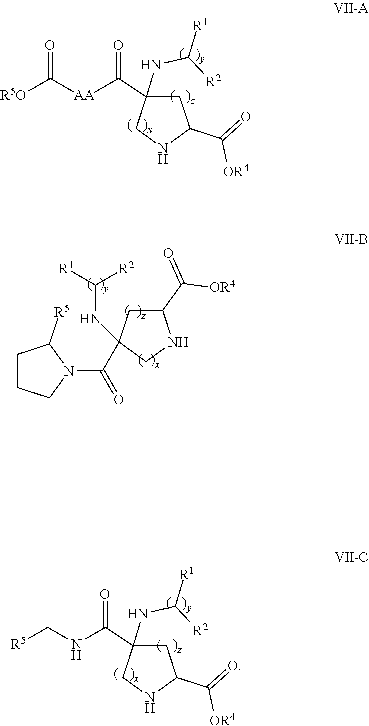

28. The process of claim 27, wherein the resin comprises a cleavable group.

29. The process of claim 28, wherein the cleavable group is selected from the group consisting of OH, carboxyl, and polystyrene resin.

30. The process of claim 27, wherein the activating agent is selected from the group consisting of 1-cyano-2-ethoxy-2-oxoethylidenaminooxy)dimethylamino-morpholino-carbenium hexafluorophosphate (COMU), 1H-1,2,3-benzotriazol-1-yloxy)-tris(pyrrolidino)-phosphonium hexafluorophosphate (PyBOP), 1-hydroxy-7-azabenzotriazole (HOAt), 2-(1H-7-azabenzotriazol-1-yl)-1,1,3-tetramethyl uronium hexafluorophosphate (HATU), benzotriazol-1-yl-oxy-tris(dimethylamino)phosphonium hexafluorophosphate (BOP), chloro-N,N,N′,N′-tetramethylformamidinium hexafluorophosphate, dicyclohexylcarbodiimide (DCC), diisopropylcarbodiimide (DIC), ethyl cyano (hydroxyimino) acetato-O²]tri-1-pyrrolidinylphosphonium hexafluorophosphate (PyOxim), ethyl cyanohydroxyiminoacetate (Oxyma), N,N′-diisopropylcarbodiimide (DIC), N-hydroxybenzotriazole (HOBT), O-(1H-benzotriazole-1-yl)-N,N,N′,N′-tetramethyluronium tetrafluoroborate (TBTU), O-1,1,3,3-tetramethyluronium hexafluorophosphate (HCTU), O-benzotriazole-N,N,N′,N′-tetramethyl uronium hexafluorophosphate (HBTU), and any combinations thereof.

31. The process of claim 27, wherein the deprotectant is a base.

32. The process of claim 31, wherein the base is selected from the group consisting of piperidine, 4-methylpiperidine, piperazine, 1,8-diazabicyclo[5.4.0]undec-7-ene (DBU), and morpholine.

33. The process of claim 27, wherein the amino acid is a natural amino acid.

34. The process of claim 33, wherein the natural amino acid is selected from the group consisting of arginine (Arg), histidine (His), lysine (Lys), aspartic acid (Asp), glutamic acid (Glu), serine (Ser), threonine (Thr), asparagine (Asn), glutamine (Gln), cysteine (Cys), selecocysteine (Sec), glycine (Gly), proline (Pro), alanine (Ala), valine (Val), isoleucine (Ile), leucine (Leu), methionine (Met), phenylalanine (Phe), tyrosine (Tyr), tryptophan (Trp), and pyrrolysine.

35. The process of claim 27, wherein the amino acid is selected from the group consisting of 4-aminobenzoic acid (PABA), alloisoleucine, allothreonine, carboxyglutamic acid, cystathionine, D-alanine, dehydroalanine, D-glutamate, diaminopimelic acid, djenkolic acid, glycine betaine homocysteine, homonorleucine, homoserine, hydroxyglycine, hydroxyproline, hypusine, isoserine, isovaline, lanthionine, N-ethyl alanine, N-ethyl glycine, N-ethyl β-alanine, N-isopropyl glycine, N-methyl alanine, N-methyl β-alanine, norleucine, norvaline, N-propyl glycine, O-methyl-homoserine, ornithine, pipecolic acid, pyroglutamic acid, sarcosine, selenocysteine, selenohomocysteine, selenomethionine, selenoethionine, taurine, t-leucine, α,β-diaminopropionic acid, α,γ-diaminobutyric acid, α-aminoisobutyric acid, α-amino-n-butyric acid, α-amino-n-heptanoic acid, α-hydroxy-γ-aminobutyric acid, β-alanine, β-aminoisobutyric acid, β-amino-n-butyric acid, γ-aminobutyric acid, δ-aminolevulinic acid, 1-aminocyclopropane-1-carboxylic acid, azetidine-2-carboxylic acid, cycloleucine, and pseudoproline.

36. The process of claim 27, wherein the compound of Formula VII is a compound having the structure of Formula VII-A, VII-B, or VII-C:

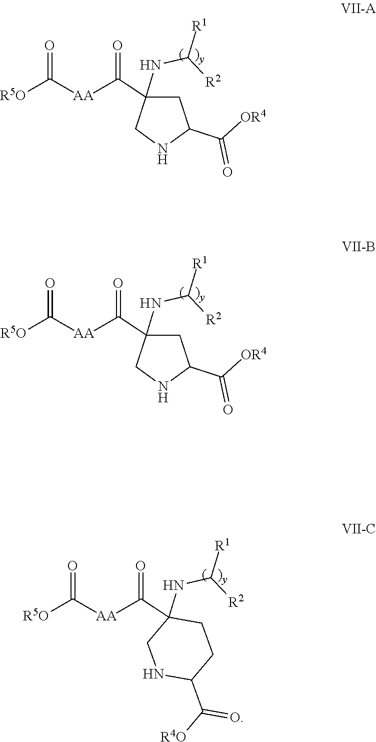

37. The process of claim 27, further comprising contacting the compound of Formula VII-A, VII-B, or VII-C with the compound of Formula II and the activating agent, followed by the deprotectant for a time and under conditions effective to produce the compound of Formula VIII-A, VIII-B, or VIII-C, respectively:

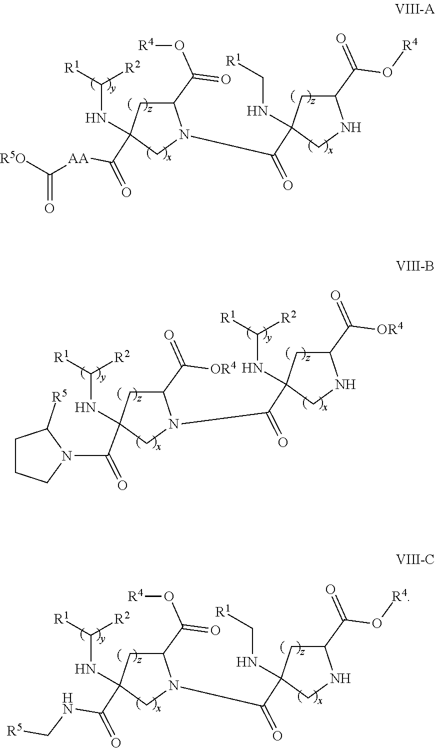

38. The process of claim 37, further comprising contacting the compound of Formula VIII-A, VIII-B, or VIII-C with the compound of Formula II and the activating agent, followed by the deprotectant for a time and under conditions effective to produce the compound of Formula IX-A, IX-B, or IX-C, respectively:

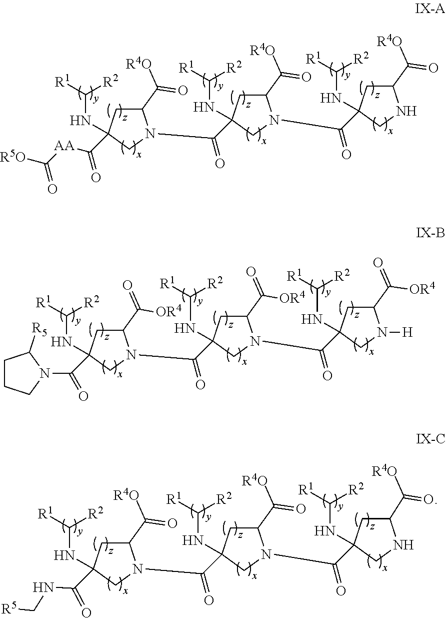

39. The process of claim 38, further comprising contacting the compound of Formula IX-A, IX-B, or IX-C with the compound of Formula II and the activating agent, followed by the deprotectant for a time and under conditions effective to produce the compound of Formula X-A, X-B, or X-C, respectively:

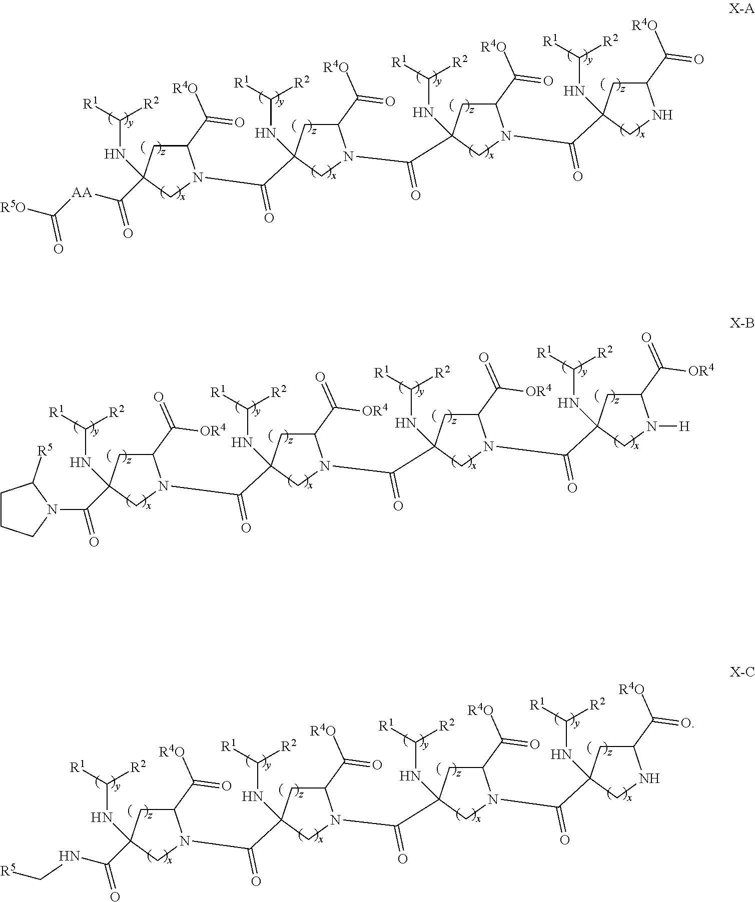

40. The process of claim 39, further comprising contacting the compound of Formula X-A, X-B, or X-C with the activating agent and Y-Z,
wherein Y is an aminocarbonyl group; and
Z is a leaving group,
followed by the deprotectant, for a time and under conditions effective to produce the compound of Formula XI-A, XI-B, or XI-C, respectively:

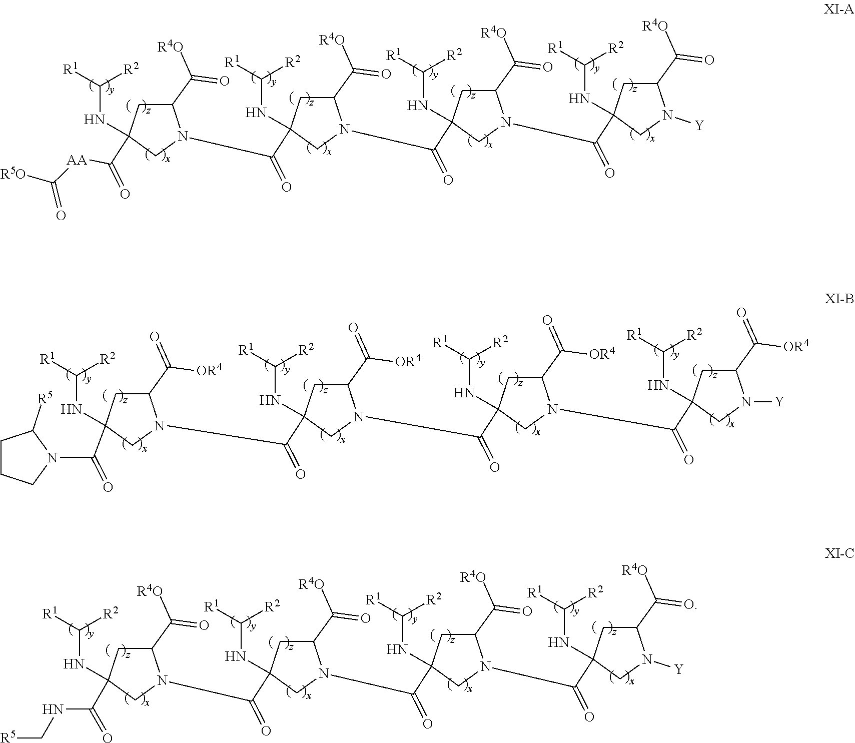

41. The process of claim 40, wherein Y is selected from the group consisting of H and an amino acid.

42. The process of claim 40, wherein Z is selected from the group consisting of a halogen, sulfonate, and triazolyl.

43. The process of claim 40, wherein Z is selected from the group consisting of chloro, fluoro, bromo, p-toluenesulfonate (OTs), methanesulfonate (OMs), trifluoromethanesulfonate (OTf), N-hydroxybenzotriazolyl, 1-hydroxy-7-azabenzotriazolyl, and

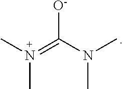

44. The process of claim 40, further comprising contacting the compound of Formula XI-A with a weak acid for a time and under conditions sufficient to produce a compound of Formula XII-A:

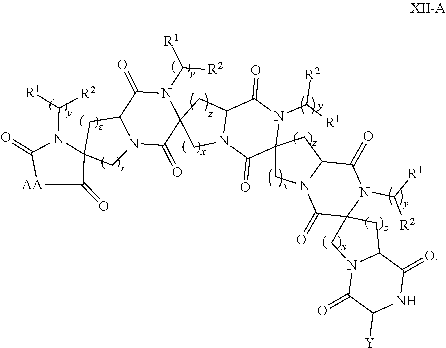

45. The process of claim 44, wherein the time is between about 1 minute to about 24 hours.

46. The process of claim 45, wherein the time is between about 2 to about 12 hours.

47. The process of claim 44, wherein the conditions sufficient to produce a compound of Formula XII-A comprise a temperature is between about room temperature to an elevated temperature.

48. The process of claim 47, wherein the elevated temperature is at least about 60° C.

49. The process of claim 44, wherein the weak acid is selected from the group consisting of trifluoroacetic acid, acetic acid, and any combination thereof.

50. The process of claim 39, comprising repeating the contacting step w times to provide a compound of Formula XV-A, XV-B, or XV-C: 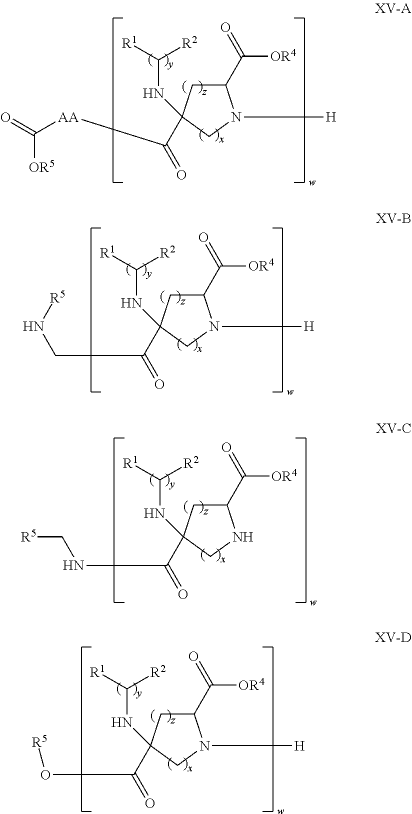
wherein, w is an integer from 1 to 20.

51. The process of claim 50, wherein w is an integer from 1 to 8.

52. The process of claim 51, wherein w is an integer from 6 to 8.

53. A compound produced using the process of claim 1.

54. The compound of claim 53, wherein the compound is a compound having the structure of Formula XIV:

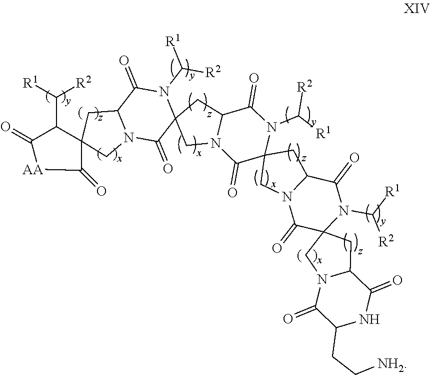

55. A process for preparing a compound of Formula I: 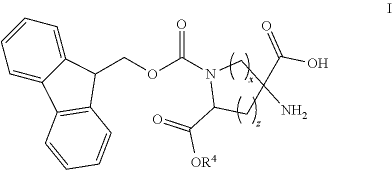
wherein R⁴ is a protecting group;
x is an integer 1 or 2; and
z is an integer 1 or 2;
with the proviso that both x and z are not 2;
comprising contacting a compound of Formula III: 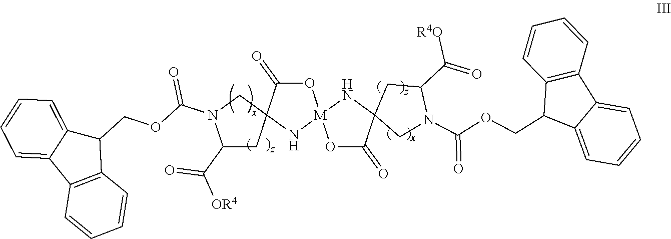
wherein M is a transition metal with a +2 oxidation state;
with a chelator for a time and under conditions effective to produce the compound of Formula I.

56. A process for preparing a compound of Formula III: 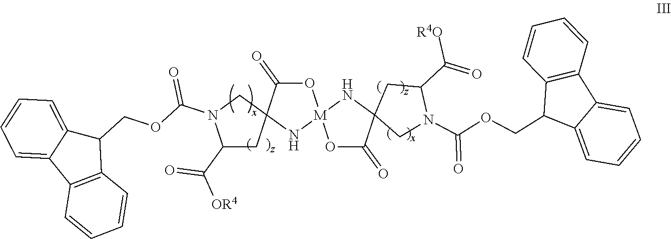
wherein R⁴ is a protecting group;
x is an integer 1 or 2; and
z is an integer 1 or 2;
with the proviso that both x and z are not 2;
comprising contacting a compound of Formula IV:
with 9-fluorenylmethoxycarbonyl chloride; for a time and under conditions effective to produce the compound of Formula III.

57. A process for preparing a compound of Formula IV: 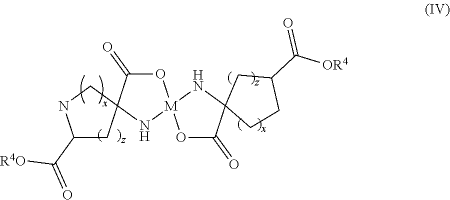
comprising contacting a compound of Formula V: 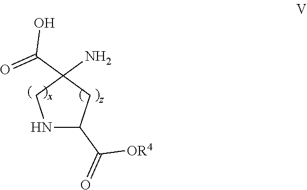
with a metal (II) source;
for a time and under conditions effective to produce the compound of Formula IV.

58. A process for preparing a compound of Formula VII: 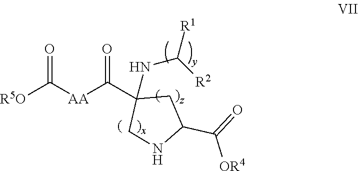
comprising contacting a compound of Formula II: 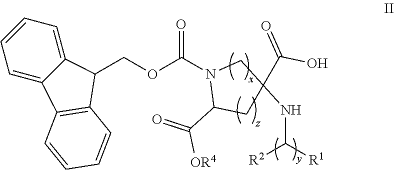
with a compound of Formula VI: 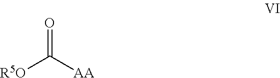
wherein AA is an optionally substituted amino acid; and
R⁵ is a resin;
with an activating agent and a base to provide an intermediate; and
contacting the intermediate with a deprotectant for a time and under conditions effective to produce the compound of Formula VII.

59. A process for preparing a compound of Formula XV: 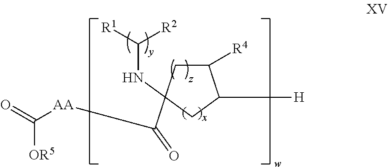
wherein R¹ is selected from the group consisting of optionally substituted C₁₋₆ alkyl, optionally substituted C₂₋₆ alkenyl, optionally substituted C₂₋₆ alkynyl, optionally substituted aryl, and any combination thereof;
R² is absent or selected from the group consisting of H, optionally substituted C₁₋₆ alkyl, optionally substituted C₂₋₆ alkenyl, optionally substituted C₂₋₆ alkynyl, optionally substituted aryl, and any combination thereof;
or R¹ and R² are linked to form a C₃₋₈ cycloalkyl;
R⁴ is a protecting group;
x is an integer 1 or 2; and
z is an integer 1 or 2, with the proviso that both x and z are not 2;
w is an integer 1 to 20; and
y is an integer 0 or 1;
comprising:
a) contacting a compound of Formula II with a compound of Formula VI: 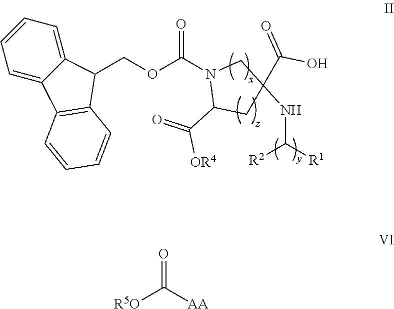
wherein AA is an optionally substituted amino acid; and
R⁵ is a resin; and
with an activating agent and a base to provide an intermediate;
b) contacting the intermediate with a deprotectant for a time and under conditions effective to produce the compound of Formula VII: 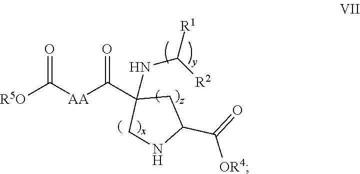
and
c) contacting the compound of Formula VII with the compound of Formula II (w-1) times.

60. A compound of Formula II: 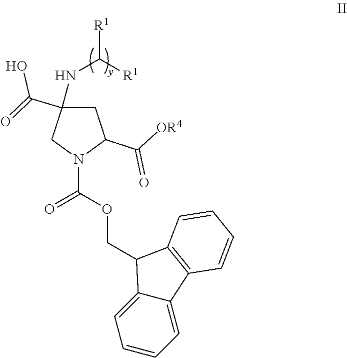
wherein y is an integer 0 or 1;
R¹ is selected from the group consisting of optionally substituted C₁₋₆ alkyl, optionally substituted C₂₋₆ alkenyl, optionally substituted C₂₋₆ alkynyl, optionally substituted aryl, and any combination thereof;
R² is absent or selected from the group consisting of H, optionally substituted C₁₋₆ alkyl, optionally substituted C₂₋₆ alkenyl, optionally substituted C₂₋₆ alkynyl, optionally substituted aryl, and any combination thereof;
or R¹ and R² are linked to form a C₃₋₈ cycloalkyl.
R⁴ is a protecting group;
or a pharmaceutically acceptable salt thereof.

61. The compound of claim 60, wherein R¹ and R² are, independently, substituted with C₀₋₆ alkylOH, C₀₋₆ alkylSH, C₀₋₆ alkylNH₂, C₀₋₆ alkyl-O—C₀₋₆ alkyl, C₀₋₆ alkyl-S—C₀₋₆ alkyl, C₀₋₆ alkylC(O)OH, C₀₋₆ alkylC(O)(C₁₋₆ alkyl), C₀₋₆ alkylC(O)O(C₁₋₆ alkyl), C₀₋₆ alkylN₃, C₀₋₆ alkylC(O)NH₂, C₀₋₆ alkyl-C(O)N(C₁₋₆ alkyl)OH, optionally substituted C₃₋₇ cycloalkyl, optionally substituted C₅₋₇ cycloalkenyl, optionally substituted aryl, optionally substituted heteroaryl, optionally substituted heterocyclyl, —NHC(NH₂)(═N(C₀₋₆ alkyl)), —NHC(NH₂)(═S(C₀₋₆ alkyl)), a nucleobase, or an amino acid;
wherein any carbon atom of said alkyl, alkenyl, or alkynyl is optionally replaced by a heteroatom selected from the group consisting of O, S, SO, SO₂, and NR⁷;
R⁷ is selected from the group consisting of H, C₁₋₄ alkyl, C₃₋₄ alkenyl, C₃₋₄ alkynyl, C₁₋₄ bridging alkyl, and any combination thereof, wherein a bridge is formed between the nitrogen and a carbon atom of said heteroatom-containing chain to form a ring, and wherein said ring is optionally fused to Ar¹;
Ar¹ is selected from the group consisting of optionally substituted C₃₋₆ cycloalkyl, heterocyclyl, aryl, heteroaryl, and any combination thereof.

62. The compound of claim 61, wherein Ar¹ is selected from the group consisting of phenyl, 1-naphthyl, 2-naphthyl, indenyl, azulenyl, fluorenyl, and anthracenyl; or a heterocyclic aromatic group selected from the group consisting of 2-furyl, 3-furyl, 2-thienyl, 3-thienyl, 2-pyridyl, 3-pyridyl, 4-pyridyl, pyrrolyl, oxazolyl, thiazolyl, imidazolyl, pyrazolyl, 2-pyrazolyl, pyrazolidinyl, isoxazolyl, isotriazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, benzo[b]thiophenyl, 1H-indazolyl, benzimidazolyl, benzthiazolyl, purinyl, 4 H-quinolizinyl, quinolinyl, 1,2,3,4-tetrahydroquinolinyl, isoquinolinyl, 1,2,3,4-tetrahydroisoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, and any combination thereof.

63. The compound of claim 61, wherein Ar¹ is optionally substituted with one or more of H, halogen, OH, NO₂, —SO₃H, CF₃, OCF₃, C₁₋₆ alkyl, C₂₋₆ alkenyl, C₁₋₆ alkoxy, O—C₃₋₄ alkenyl, —O-benzyl, —O-phenyl, 1,2-methylenedioxy, —NR⁵R⁶, —C(O)OH, —C(O)NH(C₁₋₆ alkyl), —C(O)NH(C₃₋₅alkenyl), —C(O)N(C₁₋₆ alkyl)(C₁₋₆ alkyl), —C(O)(C₃₋₅alkenyl)(C₃₋₆ alkenyl), morpholinyl, piperidinyl, —O—Ar², —CH₂—(CH₂)_{q}—Ar², —O—(CH₂)_{q}—Ar², —(CH₂)_{q}—O—Ar², or —CH═CH—Ar²;
R⁵ and R⁶ are, independently, selected from the group consisting of H, C₁₋₆ alkyl, C₃₋₆ alkenyl, C₃₋₆ alkynyl, and benzyl;
Ar² is selected from the group consisting of 4-methoxyphenyl, 2-pyridyl, 3-pyridyl, 4-pyridyl, pyrazyl, quinolyl, 3,5-dimethylisoxazoyl, 2-methylthiazoyl, thiazolyl, 2-thienyl, 3-thienyl, and pyrimidinyl; and
q is an integer between 0 to 2.

64. The compound of claim 60, wherein one or both of R¹ and R² are selected from the group consisting of an amino acid, β-amino acid, and sugar.

65. The compound of claim 60, wherein R⁴ is C₁₋₆ alkyl.

66. The compound of claim 60, wherein R⁴ is selected from the group consisting of t-butyl and 4-{N-[1-(4,4-dimethyl-2,6-dioxocyclohexylidene)-3-methylbutyl]amino}benzyl ester (DMab).

67. The compound of claim 60, wherein the compound of Formula II is a compound having the structure of Formula II-A, II-B, or II-C:

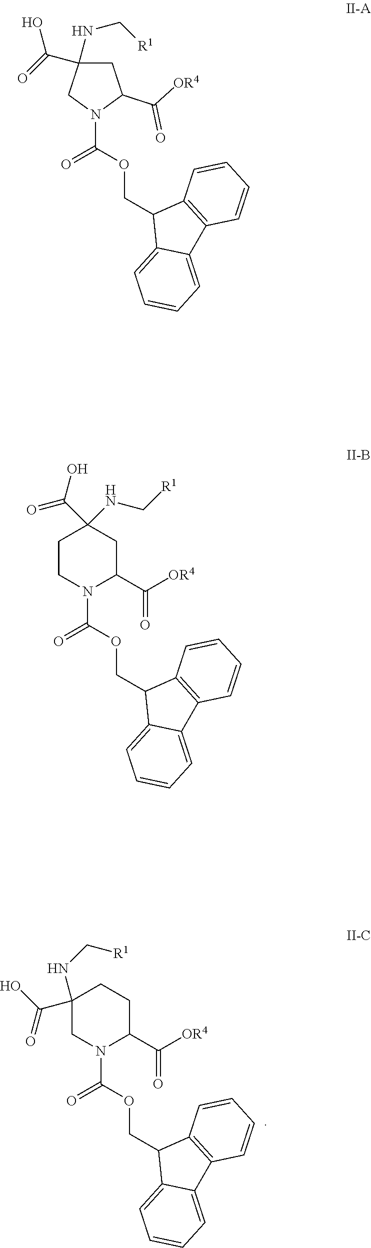

68. A compound having the structure of:

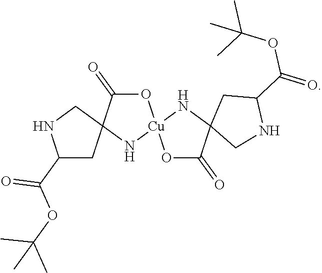

69. A compound having the structure of:

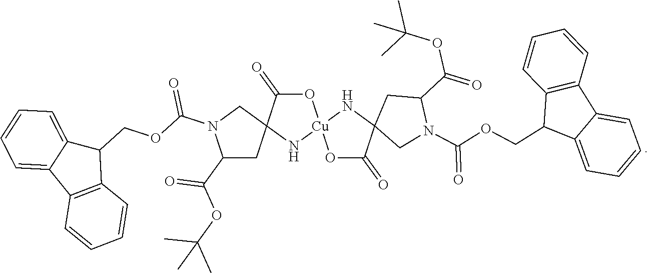

70. A compound having the structure of: 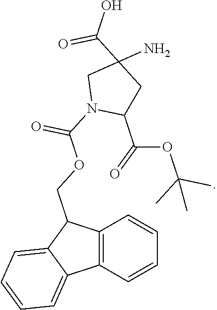 [SMILES: CC(C)(C)OC(=O)C1CC(N)(C(=O)O)CN1C(=O)OCC1c2ccccc2-c2ccccc21]
